(19) United States
(12) Patent Application Publication
(10) Pub. No.: US 2005/0059726 A1
(43) Pub. Date: Mar. 17, 2005

(54) Pyrrolidine-2-ones as factor xa inhibitors

(76) Inventors: Alan David Borthwick, Stevenage (GB); Chuen Chan, Stevenage (GB); Henry Kelly, Stevenage (GB); Nigel King, Stevenage (GB); Savvas Kleanthous, Stevenage (GB); Andrew Mason, Stevenage (GB); Ivan Pinto, Stevenage (GB); Derek Pollard, Stevenage (GB); Stefan Senger, Stevenage (GB); Gita Shah, Stevenage (GB); Nigel Watson, Stevenage (GB); Robert Young, Stevenage (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/499,529

(22) PCT Filed: Dec. 20, 2002
(86) PCT No.: PCT/EP02/14826

(30) Foreign Application Priority Data
Dec. 21, 2001 (GB) 0130705.7

(51) Int. Cl. A61K 31/404; A61K 31/4025; C07D 49/02; C07D 45/02; C07D 43/02

(52) U.S. Cl. 514/414; 514/422; 514/424; 548/465; 548/525; 548/527

(57) ABSTRACT

The present invention relates to novel compounds and pharmaceutically acceptable derivatives thereof. The invention also relates to processes for the preparation of the compounds, pharmaceutical compositions containing the compounds, and to the use of compounds in medicine, particularly in the amelioration of a clinical condition for which a Factor Xa inhibitor is indicated.

### FIELD OF THE INVENTION

The present invention relates to a novel class of chemical compounds, to processes for their preparation, to pharmaceutical compositions containing them and to their use in medicine, particularly use in the amelioration of a clinical condition for which a Factor Xa inhibitor is indicated.

### BACKGROUND OF THE INVENTION

Factor Xa is a member of the trypsin-like serine protease class of enzymes. It is a key enzyme in the coagulation cascade. A one-to-one binding of Factors Xa and Va with calcium ions and phospholipid converts prothrombin into thrombin. Thrombin plays a central role in the mechanism of blood coagulation by converting the soluble plasma protein, fibrinogen, into insoluble fibrin. The insoluble fibrin matrix is required for the stabilisation of the primary hemostatic plug. Many significant disease states are related to abnormal hemostasis. With respect to the coronary arterial vasculature, abnormal thrombus formation due to the rupture of an established atherosclerotic plaque is the major cause of acute myocardial infarction and unstable angina. Both treatment of an occlusive coronary thrombus by thrombolytic therapy and percutaneous transluminal coronary angioplasty (PTCA) are often accompanied by an acute thrombotic reclosure of the affected vessel which requires immediate resolution. With respect to the venous vasculature, a high percentage of patients undergoing major surgery in the lower extremities or the abdominal area suffer from thrombus formation in the venous vasculature which can result in reduced blood flow to the affected extremity and a pre-disposition to pulmonary embolism. Disseminated intravascular coagulopathy commonly occurs within both vascular systems during septic shock, certain viral infections and cancer and is characterised by the rapid consumption of coagulation factors and systemic coagulation which results in the formation of life-threatening thrombi occurring throughout the vasculature leading to widespread organ failure. Beyond its direct role in the formation of fibrin rich blood clots, thrombin has been reported to have profound bioregulatory effects on a number of cellular components within the vasculature and blood, (Shuman, M. A., Ann. NY Acad. Sci., 405: 349 (1986)).

A Factor Xa inhibitor may be useful in the treatment of acute vascular diseases such as coronary thrombosis (for example myocardial infarction and unstable angina), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke. They may also have utility as anti-coagulant agents both in-vivo and ex-vivo, and in oedema and inflammation. Thrombin has been reported to contribute to lung fibroblast proliferation, thus, Factor Xa inhibitors could be useful for the treatment of some pulmonary fibrotic diseases. Factor Xa inhibitors could also be useful in the treatment of tumour metastasis, preventing the fibrin deposition and metastasis caused by the inappropriate activation of Factor Xa by cysteine proteinases produced by certain tumour cells. Thrombin can induce neurite retraction and thus Factor Xa inhibitors may have potential in neurogenerative diseases such as Parkinson's and Alzheimer's disease. They have also been reported for use in conjunction with thrombolytic agents, thus permitting the use of a lower dose of thrombolytic agent.

### DESCRIPTION OF THE INVENTION

The present invention provides compounds of formula (I): 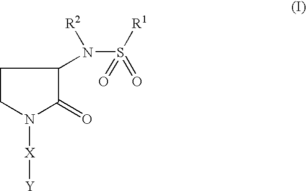
wherein:
R¹ represents a group selected from: 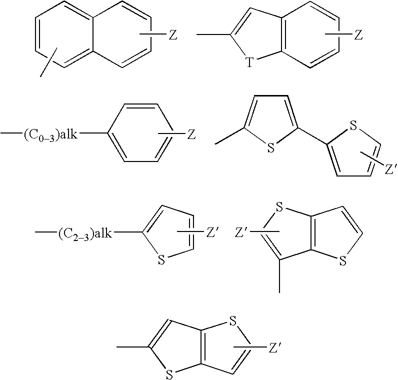
   each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alkylene or alkenylene,
T represents S, O or NH;
R² represents hydrogen, —C₁₋₃alkylCONR^{a}R^{b}, —C₁₋₃alkylCO₂C₁₋₄alkyl, —C₁₋₃alkylmorpholino, —CO₂C₁₋₄alkyl, or —C₁₋₃alkylCO₂H;
X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, and —S(O)₂NR^{a}R^{b};
Y represents (i) a substituent selected from hydrogen, halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, or —S(O)₂NR^{a}R^{b}, or (ii) phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)R^{c}, —C(O)NR^{a}R^{b}, —S(O)ₙR^{c}, —S(O)₂NR^{a}R^{b}, ═O, oxide to a ring N, —CHO, —NO₂, and —N(R^{a})(SO₂R^{c});
R^{a} and R^{b} independently represent hydrogen, —C₁₋₆alkyl, or together with the N atom to which they are bonded form a 5-, 6- or 7-membered heterocyclic ring optionally containing an additional heteroatom selected from O, N or S, optionally substituted by C₁₋₄alkyl, and optionally the S heteroatom is substituted by O i.e. represents S(O)ₙ;
R^{c} represents —C₁₋₆alkyl;
R^{d} represents hydrogen or —C₁₋₆alkyl;
n represents 0-2;
and pharmaceutically acceptable derivatives thereof.

Further aspects of the invention are:
A pharmaceutical composition comprising a compound of the invention together with a pharmaceutical carrier and/or excipient.
A compound of the invention for use in therapy.
Use of a compound of the invention for the manufacture of a medicament for the treatment of a patient suffering from a condition susceptible to amelioration by a Factor Xa inhibitor.
A method of treating a patient suffering from a condition susceptible to amelioration by a Factor Xa inhibitor comprising administering a therapeutically effective amount of a compound of the invention.

The present invention also provides compounds of formula (I) wherein:
R¹ represents a group selected from: 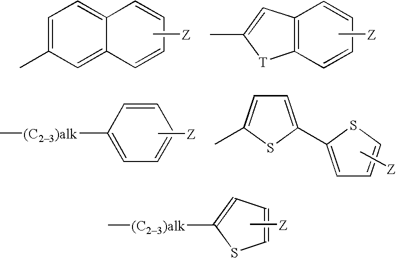
Z represents an optional substituent halogen,
alk represents alkylene or alkenylene,
T represents S, O or NH;
R² represents hydrogen;
X represents phenyl or a 5 or 6 membered aromatic or non-aromatic heterocyclic group containing at least one heteroatom selected from O, N or S, each of which is optionally substituted by 0-2 groups selected from: halogen —CN, —C₁₋₄alkyl, —CF₃, —NR^{a}R^{b}, —(CH₂)ₙOR^{c}, —C(O)R^{c}, —C(O)NR^{a}R^{b}, —S(O)ₙR^{c}, —S(O)₂NR^{a}R^{b};
Y represents (i) a substituent selected from hydrogen, halogen —CN, —C₁₋₄alkyl, —CF₃, —NR^{a}R^{b}, —(CH₂)ₙOR^{c}, —C(O)R^{c}, —C(O)NR^{a}R^{b}, —S(O)ₙR^{c}, —S(O)₂NR^{a}R^{b} or (ii) phenyl or a 5 or 6 membered aromatic or non-aromatic heterocyclic group containing at least one heteroatom selected from O, N or S, each of which is optionally substituted by 0-2 groups selected from: halogen —CN, —C₁₋₄alkyl, —CF₃, (CH₂)ₙNR^{a}R^{b}, —(CH₂)ₙOR^{c}, —C(O)R^{c}, —C(O)NR^{a}R^{b}, —S(O)ₙR^{c}, —S(O)₂NR^{a}R^{b};
R^{a} and R^{b} independently represent hydrogen, —C₁₋₆alkyl or together with the N atom to which they are bonded form a 5-, 6- or 7-membered heterocyclic ring optionally containing an additional heteroatom selected from O, N or S and are optionally substituted by C₁₋₄alkyl, optionally the S heteroatom is substituted by O i.e. represents S(O)ₙ;
R^{c} represents —C₁₋₆alkyl;
n represents 0-2;
and pharmaceutically acceptable salts or solvates thereof.

In another aspect, the present invention provides a compound of formula (I) having a formula (IA): 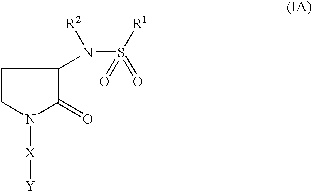
wherein:
R¹ represents a group selected from: 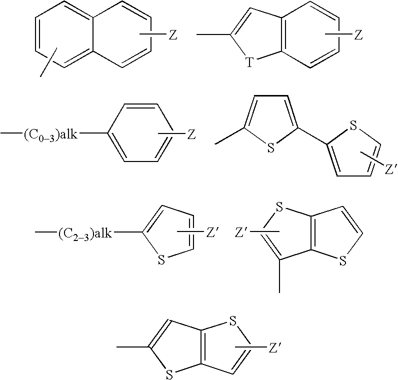
   each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alkylene or alkenylene,
T represents S, O or NH;
R² represents hydrogen, —C₁₋₃alkylCONR^{a}R^{b}, —C₁₋₃alkylCO₂C₁₋₄alkyl, —C₁₋₃alkylmorpholino, —CO₂C₁₋₄alkyl, or —C₁₋₃alkylCO₂H;
X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, and —S(O)₂NR^{a}R^{b};
Y represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)R^{c}, —C(O)N³R^{b}, —S(O)ₙR^{c}, —S(O)₂NR^{a}R^{b}, ═O, oxide to a ring N, —CHO, —NO₂, and —N(R^{a})(SO₂R^{c});
R^{a} and R^{b} independently represent hydrogen, —C₁₋₆alkyl, or together with the N atom to which they are bonded form a 5-, 6- or 7-membered heterocyclic ring optionally containing an additional heteroatom selected from O, N or S, optionally substituted by C₁₋₄alkyl, and optionally the S heteroatom is substituted by O i.e. represents S(O)ₙ;
R^{c} represents —C₁₋₆alkyl;
R^{d} represents hydrogen or —C₁₋₆alkyl;
n represents 0-2;
and pharmaceutically acceptable derivatives thereof.

The present invention also provides compounds of formula (IA) wherein:
R¹ represents a group selected from: 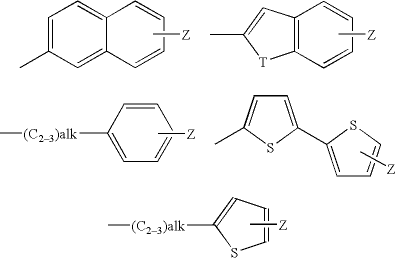
Z represents an optional substituent halogen,
alk represents alkylene or alkenylene,
T represents S, O or NH;
R² represents hydrogen;
X represents phenyl optionally substituted by 0-2 groups selected from: halogen —CN, —C₁₋₄alkyl, —CF₃, —NR^{a}R^{b}, —(CH₂)ₙOR^{c}, —C(O)R^{c}, —C(O)NR^{a}R^{b}, —S(O)ₙR^{c}, —S(O)₂NR^{a}R^{b};
Y represents phenyl optionally substituted by 0-2 groups selected from: halogen —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, —S(O)₂NR^{a}R^{b};
R^{a} and R^{b} independently represent hydrogen, —C₁₋₆alkyl or together with the N atom to which they are bonded form a 5-, 6- or 7-membered heterocyclic ring optionally containing an additional heteroatom selected from O, N or S and are optionally substituted by C₁₋₄alkyl, optionally the S heteroatom is substituted by O i.e. represents S(O)ₙ;
R^{c} represents —C₁₋₆alkyl;
n represents 0-2;
and pharmaceutically acceptable salts and solvates thereof.

In another aspect, the present invention provides a compound of formula (I) having a formula (IB): 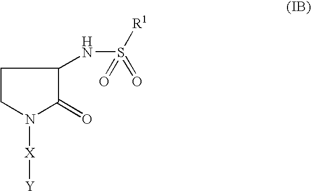
wherein:
R¹ represents a group selected from: 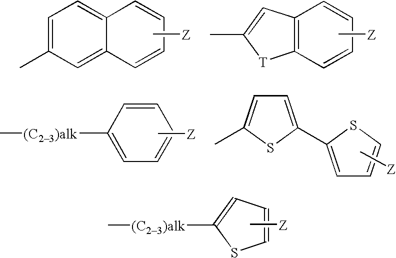
Z represents an optional substituent halogen,
alk represents alkylene or alkenylene,
T represents S, O or NH;
X represents phenyl optionally substituted by 0-2 groups selected from: halogen —CN, —C₁₋₄alkyl, —CF₃, —(CH₂)ₙOR^{c}, —C(O)R^{c}, —C(O)NR^{a}R^{b}, S(O)ₙR^{c}, —S(O)₂NR^{a}R^{b};
Y represents —NR^{a}R^{b};
R^{a} and R^{b} independently represent hydrogen, —C₁₋₆alkyl or together with the N atom to which they are bonded from a 5-, 6- or 7-membered heterocyclic ring optionally containing an additional heteroatom selected from O, N or S and are optionally substituted by C₁₋₄alkyl, optionally the S heteroatom is substituted by O i.e. represents S(O)ₙ;
R^{c} represents —C₁₋₆alkyl;
n represents 0-2;
and pharmaceutically acceptable salts and solvates thereof.

In another aspect, the present invention provides a compound of formula (I) having a formula (IC): 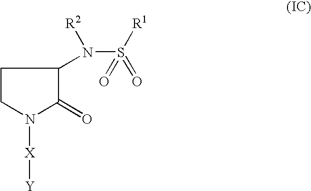
wherein:
R¹ represents a group selected from: 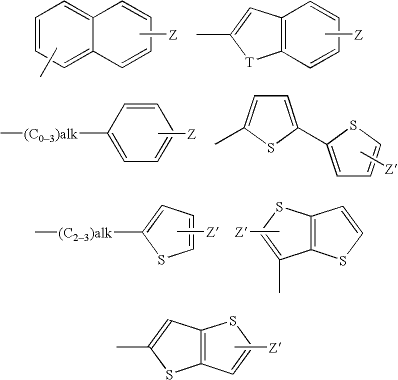
   each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alkylene or alkenylene,
T represents S, O or NH;
R² represents hydrogen, —C₁₋₃alkylCONR^{a}R^{b}, —C₁₋₃alkylCO₂C₁₋₄alkyl, —C₁₋₃alkylmorpholino, —CO₂C₁₋₄alkyl), or —C₁₋₃alkylCO₂H;
X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, and —S(O)₂NR^{a}R^{b};
Y represents a substituent selected from hydrogen, halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, or —S(O)₂NR^{a}R^{b};
R^{a} and R^{b} independently represent hydrogen, —C₁₋₆alkyl, or together with the N atom to which they are bonded form a 5-, 6- or 7-membered heterocyclic ring optionally containing an additional heteroatom selected from O, N or S, optionally substituted by C₁₋₄alkyl, and optionally the S heteroatom is substituted by O i.e. represents S(O)ₙ;
R^{c} represents —C₁₋₆alkyl;
R^{d} represents hydrogen or —C₁₋₆alkyl;
n represents 0-2;
and pharmaceutically acceptable derivatives thereof.

The present invention also provides compounds of formula (IC) wherein:
R¹ represents a group selected from: 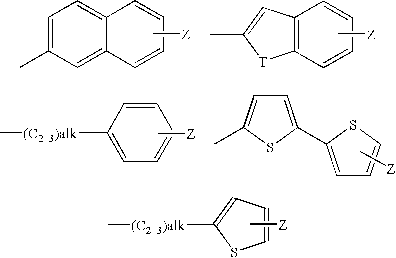
Z represents an optional substituent halogen,
alk represents alkylene or alkenylene,
T represents S, O or NH;
R² represents hydrogen;
X represents phenyl or a 5 or 6 membered aromatic or non-aromatic heterocyclic group containing at least one heteroatom selected from O, N or S, each of which is optionally substituted by 0-2 groups selected from: halogen —CN, —C₁₋₄alkyl, —CF₃, —NR^{a}R^{b}, —(CH₂)ₙOR^{c}, —C(O)R^{c}, —C(O)NR^{a}R^{b}, —S(O)ₙR^{c}, —S(O)₂NR^{a}R^{b};
Y represents a substituent selected from hydrogen, halogen —CN, —C₁₋₄alkyl, —CF₃, —NR^{a}R^{b}, —(CH₂)ₙOR^{c}, —C(O)R^{c}, —C(O)NR^{a}R^{b}, —S(O)ₙR^{c}, —S(O)₂NR^{a}R^{b};
R^{a} and R^{b} independently represent hydrogen, —C₁₋₆alkyl or together with the N atom to which they are bonded form a 5-, 6- or 7-membered heterocyclic ring optionally containing an additional heteroatom selected from O, N or S and are optionally substituted by C₁₋₄alkyl, optionally the S heteroatom is substituted by O i.e. represents S(O)ₙ;
R^{c} represents —C₁₋₄alkyl;
n represents 0-2;
and pharmaceutically acceptable salts or solvates thereof.

In another aspect, the present invention provides compounds of formula (I) wherein X and Y are as defined above and R¹ represents chloronaphthylene, preferably 6-chloronaphthylene.

The compounds of formula (I) contain chiral (asymmetric) centres. The individual stereoisomers (enantiomers and diastereoisomers) and mixtures of these are within the scope of the present invention.

In a compound of formula (I):

Preferably, R¹ represents a group selected from: 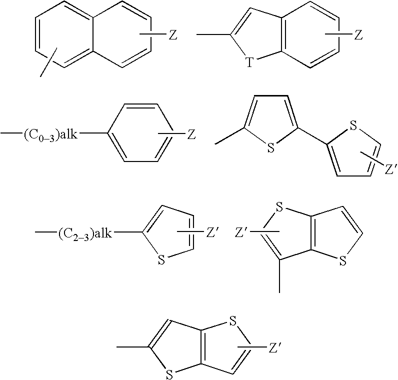
each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alkylene or alkenylene,
T represents S or O.

More preferably, R¹ represents a group selected from: 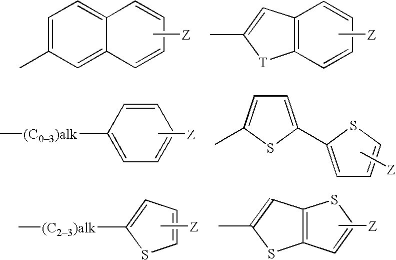
each of which optionally contain a further heteroatom N,
Z represents an optional substituent halogen,
alk represents alkylene or alkenylene,
T represents S or O.

Even more preferably, R¹ represents a group selected from:

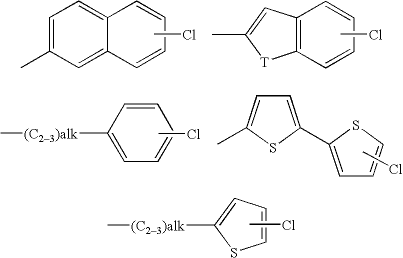

Most preferably, R¹ represents:

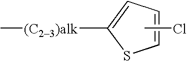

Preferably, R² represents hydrogen, CH₂CONH₂, CH₂CO₂CH₃, CH₂CO₂C₄alkyl, CH₂CO₂H, CO₂C₄alkyl, (CH₂)₂morpholino. Preferably, R² represents hydrogen or CH₂CONH₂.

Preferably, when R² represents C₂H₄morpholino, the morpholino ring is N-linked to the alkyl chain.

Preferably, X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, C₂₋₄alkenyl, —NR^{a}R^{b}, —N(C₁₋₄alkyl)(CHO), —NO₂, —NHCOC₁₋₄alkyl, NH₂SO₂R^{c}, C₀₋₄alkylOR^{d}, —C(O)R^{c}, and —C(O)NR^{a}R^{b}. More preferably, X represents phenyl or a 5 or 6 membered aromatic heterocyclic group containing at least one heteroatom selected from O, N or S, each of which is optionally substituted by 0-2 groups selected from: halogen, —CN, —C₁₋₄alkyl, —C₂₋₄alkenyl, —NR^{a}R^{b}, —N(C₁₋₄alkyl)(CHO), NO₂, NHSO₂R^{c}, C₀₋₄alkylOR^{d}, —C(O)R^{c}, and —C(O)NR^{a}R^{b}. Even more preferably, X represents phenyl or a 5 or 6 membered aromatic heterocyclic group containing at least one heteroatom selected from O, N or S, each of which is optionally substituted by 0-2 groups selected from: halogen, —CN, —C₂₋₄alkenyl, —N(C₁₋₄alkyl)(CHO), —C(O)R^{c}, and —C(O)NR^{a}R^{b}. Even more preferably, X represents phenyl optionally substituted by halogen or a 5 or 6 membered aromatic heterocyclic group containing at least one heteroatom selected from O, N or S. Even more preferably, X represents phenyl substituted by hydrogen or halogen, or pyridine. Most preferably, X represents phenyl substituted at the 2-position by fluorine, or pyridine.

Preferably, Y represents (i) a substituent selected from hydrogen, halogen, —CN, C₁₋₄alkyl, —C₂₋₄alkenyl, —NR^{a}R^{b}, —N(C₁₋₄alkyl)(CHO), NO₂, —NHCOC₁₋₄alkyl, —NHSO₂R^{c}, C₀₋₄alkylOR^{d}, —C(O)R^{c}, or —C(O)NR^{a}R^{b}, or (ii) phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)NR^{a}R^{b}, —S(O)ₙR^{c}, —S(O)₂NR^{a}R^{b}, oxide to a ring N, —CHO, —NO₂, and —N(R^{a})(SO₂R^{c}). More preferably, Y represents (i) a substituent selected from hydrogen, halogen, —CN, —C₂₋₄alkenyl, —NR^{a}R^{b}, —N(C₁₋₄alkyl)(CHO), NO₂, —NHSO₂R^{c}, C₀₋₄alkyl OR^{d}, —C(O)R^{c}, or —C(O)NR^{a}R^{b}, or (ii) phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)NR^{a}R^{b}, —S(O)ₙR^{c}, —S(O)₂NR^{a}R^{b}, oxide to a ring N, —CHO, —NO₂, and —N(R^{a})(SO₂R^{c}). Even more preferably, Y represents (i) a substituent selected from hydrogen, halogen, —CN, —C₂₋₄alkenyl, —N(C₁₋₄alkyl)(CHO), —C(O)R^{c}, or —C(O)NR^{a}R^{b}, or (ii) phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, (CH₂)ₙN⁺R^{a}R^{b}CH₂CONH₂ , C₀₋₄alkylOR^{d}, —C(O)NR^{a}R^{b}, —S(O)ₙR^{c}—S(O)₂NR^{a}R^{b}, NO₂, and —N(R^{a})(SO₂R^{c}). Most preferably, Y represents (i) a substituent selected from hydrogen, halogen, —CN, —C(O)R^{c}, or —C(O)NR^{a}R^{b}, or (ii) phenyl, pyrazole, imidazole or pyridine, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)NR^{a}R^{b}, —S(O)ₙR^{c}, —S(O)₂NR^{a}R^{b}, NO₂, and —N(R^{a})(SO₂R^{c}) In another preferred aspect, Y represents (i) a substituent selected from hydrogen, halogen, —CN, —C₁₋₄alkyl, —C₂₋₄alkenyl, —CF₃, —NR^{a}R^{b}, NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c} or —S(O)₂NR^{a}R^{b}, (ii) phenyl optionally substituted by 0-2 groups selected from: halogen, —CN, —C₁₋₄alkyl, —CF₃, —(CH₂)ₙNR^{a}R^{b}, C₀₋₄alkylOR^{d}, —C(O)R^{c}, —C(O)NR^{a}R^{b}, —S(O)ₙR^{c}, —S(O)₂NR^{a}R^{b}, —CHO, —NO₂, and —N(R^{a})(SO₂R^{c}), (iii) a 5 or 6 membered aromatic or non-aromatic heterocyclic group containing at least one heteroatom selected from O, N or S, each of which is substituted by a group selected from: —S(O)ₙR^{c}, —S(O)₂NR^{a}R^{b}, —NO₂, or —N(R^{a})(SO₂R^{c}), or (iv) when R¹represents 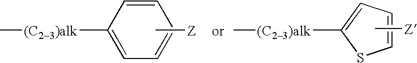
Y represents a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)R^{c}, —C(O)NR^{a}R^{b}, —S(O)ₙR^{c}, —S(O)₂NR^{a}R^{b}, —CHO, NO₂, and —N(R^{a})(SO₂R^{c}).

More preferably, Y represents (i) a substituent selected from hydrogen, halogen, —CN, —C₁₋₄alkyl, —CF₃, —, NR^{a}R^{b}, —(CH₂)ₙOR^{c}, —C(O)R^{c}, —C(O)NR^{a}R^{b}, —S(O)ₙR^{c}, or —S(O)₂NR^{a}R^{b}, (ii) phenyl optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, and —S(O)₂NR^{a}R^{b}, (iii) a 5 or 6 membered aromatic or non-aromatic heterocyclic group containing at least one heteroatom selected from O, N or S, each of which is substituted by a group selected from: —S(O)ₙR^{c}, or —S(O)₂NR^{a}R^{b}, or (iv) when R¹ represents 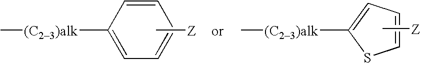
and Z represents an optional substituent halogen,
Y represents a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, —S(O)₂NR^{a}R^{b}.

Most preferably, Y represents (i) a substituent selected from hydrogen, halogen, —CN, —C₁₋₄alkyl, —CF₃, —NR^{a}R^{b}, (CH₂)ₙOR^{c}, —C(O)R^{c}, —C(O)NR^{a}R^{b}, —S(O)ₙR^{c} or —S(O)₂NR^{a}R^{b}, or (ii) phenyl optionally substituted by 0-2 groups selected from: halogen, —CN, —C₁₋₄alkyl, —CF₃, —(CH₂)ₙNR^{a}R^{b}, —(CH₂)ᵤOR^{c}, —C(O)R^{c}, —C(O)NR^{a}R^{b}, —S(O)ₙR^{c}, and —S(O)₂NR^{a}R^{b}, or (iii) when R¹ represents 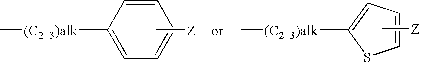
and Z represents an optional substituent halogen,
Y represents pyrazole, imidazole or pyridine, each of which is optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, —S(O)₂NR^{a}R^{b}. Preferably, when X is phenyl, Y is a substituent at the 4-position (i.e. para to the rest of the molecule) on the phenyl ring.

Preferably, R^{a} and R^{b} independently represent hydrogen or —C₁₋₆alkyl.

In a compound of formula (IA):

Preferably, R¹ represents a group selected from: 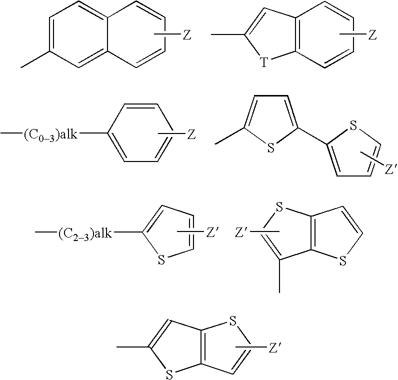
each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alklene or alkenylene,
T represents S or O.

More preferably, R¹ represents a group selected from: 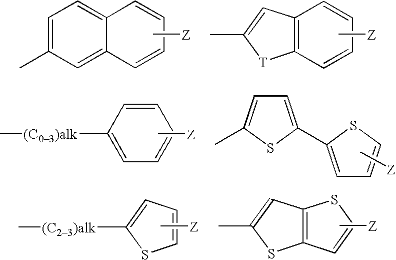
each of which optionally contain a further heteroatom N,
Z represents an optional substituent halogen,
alk represents alkylene or alkenylene,
T represents S or O.

Even more preferably, R¹ represents a group selected from:

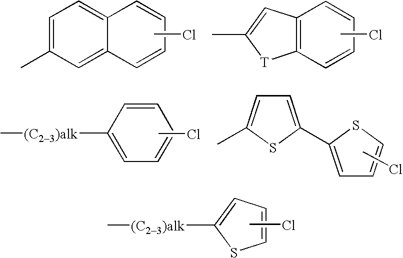

Most preferably, R¹ represents a group selected from:

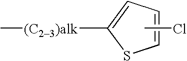

Preferably, R² represents hydrogen, CH₂CONH₂, CH₂CO₂CH₃, CH₂CO₂C₄alkyl, CH₂CO₂H, CO₂C₄alkyl, (CH₂)₂morpholino. Preferably, R² represents hydrogen or CH₂CONH₂.

Preferably, when R² represents C₂H₄morpholino, the morpholino ring is N-linked to the alkyl chain.

Preferably, X represents phenyl optionally substituted by halogen or a 5 or 6 membered aromatic heterocyclic group containing at least one heteroatom selected from O, N or S. More preferably, X represents phenyl or a 5 or 6 membered aromatic heterocyclic group containing at least one heteroatom selected from O, N or S, each of which is optionally substituted by 1-2 groups selected from: halogen. Even more preferably, X represents phenyl substituted by halogen, or pyridine. Most preferably, X represents phenyl substituted at the 2-position by fluorine, or pyridine.

Preferably, Y represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)NR^{a}R^{b}, —S(O)ₙR^{c}, —S(O)₂NR^{a}R^{b}, oxide to a ring N, —CHO, NO₂, or —N(R^{a})(SO₂R^{c}). More preferably, Y represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)NR^{a}R^{b}, —S(O)ₙR^{c}, —S(O)₂NR^{a}R^{b}, —NO₂, or —N(R^{a})(SO₂R^{c}). Most preferably, Y represents phenyl, pyridine or pyrazole optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)NR^{a}R^{b}, —S(O)ₙR^{c}, —S(O)₂NR^{a}R^{b}, NO₂, or —N(R^{a})(SO₂R^{c}). In another preferred aspect, Y represents (i) phenyl optionally substituted by 0-2 groups selected from: halogen, —CN, —C₁₋₄alkyl, —CF₃, —(CH₂)ₙNR^{a}R^{b}, C₀₋₄alkylOR^{d}, —C(O)R^{c}, —C(O)NR^{a}R^{b}, —S(O)ₙR^{c}, —S(O)₂NR^{a}R^{b}, —CHO, —NO₂, and —N(R^{a})(SO₂R^{c}), (ii) a 5 or 6 membered aromatic or non-aromatic heterocyclic group containing at least one heteroatom selected from O, N or S, each of which is substituted by a group selected from: —S(O)ₙR^{c}, —S(O)₂NR^{a}R^{b}, NO₂, or —N(R^{a})(SO₂R^{c}), or (iii) when R¹ represents 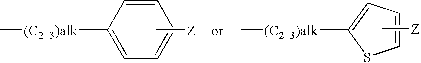
Y represents a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, C₀₋₄alkylOR^{d}, —C(O)R^{c}, —C(O)NR^{a}R^{b}, —S(O)ₙR^{c}, —S(O)₂N^{a}R^{b}, —CHO, NO₂, —N(R^{a})(SO₂R^{c}).

More preferably, Y represents (i) phenyl optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O),R^{c}, and —S(O)₂NR^{a}R^{b}, (ii) a 5 or 6 membered aromatic or non-aromatic heterocyclic group containing at least one heteroatom selected from O, N or S, each of which is substituted by a group selected from: —S(O)ₙR^{c}, or —S(O)₂NR^{a}R^{b}, or (iii) when R¹ represents 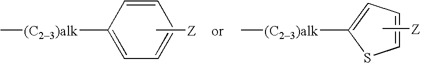
Y represents a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, and —S(O)₂NR^{a}R^{b}.

Most preferably, Y represents (i) phenyl optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, and —S(O)₂NR^{a}R^{b}, or (iii) when R¹ represents 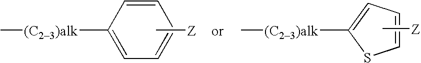
Y represents phenyl, pyrazole, imidazole or pyridine, each of which is optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, S(O)ₙR^{c}, and —S(O)₂NR^{a}R^{b}.

Preferably, when X is phenyl, Y is a substituent at the 4-position (i.e. para to the rest of the molecule) on the phenyl ring.

Preferably, R^{a} and R^{b} independently represent hydrogen or —C₁₋₆alkyl.

In a compound of formula (IC):

Preferably, R¹ represents a group selected from: 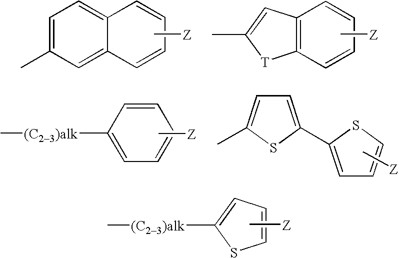
Z represents an optional substituent halogen,
T represents S.

Even more preferably, R¹ represents a group selected from: 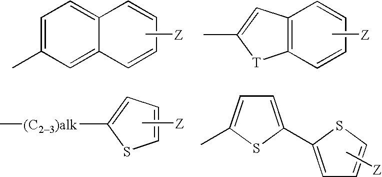
Z represents an optional substituent halogen,
T represents S.

More preferably, R¹ represents a group selected from:

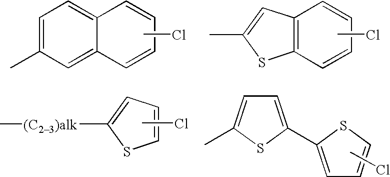

Most preferably, R¹ represents:

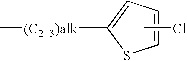

Preferably, R² represents hydrogen or CH₂CONH₂.

Preferably, X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —C₂₋₄alkenyl, —NR^{a}R^{b}, —N(C₁₋₄alkyl)(CHO), NO₂, —NHCOC₁₋₄alkyl, —NHSO₂R^{c}, C₀₋₄alkylOH, —C(O)R^{c}, and —C(O)NR^{a}R^{b}. More preferably, X represents phenyl or a 5 or 6 membered aromatic heterocyclic group containing at least one heteroatom selected from O, N or S, each of which is optionally substituted by 0-2 groups selected from: halogen, —CN, C₂₋₄alkenyl, —NR^{a}R^{b}, —N(C₁₋₄alkyl)(CHO), NO₂, C₀₋₄alkylOH, —C(O)R^{c}, and —C(O)NR^{a}R^{b}. Even more preferably, X represents phenyl or a 5 or 6 membered aromatic heterocyclic group containing at least one heteroatom selected from O, N or S, each of which is optionally substituted by 0-2 groups selected from: halogen, —CN, —N(C₁₋₄alkyl)(CHO), —C(O)R^{c}, and —C(O)NR^{a}R^{b}. Even more preferably, X represents phenyl optionally substituted by halogen or a 5 or 6 membered aromatic heterocyclic group containing at least one heteroatom selected from O, N or S. Even more preferably, X represents phenyl substituted by halogen, or pyridine. Even more preferably, X represents phenyl substituted by halogen. Most preferably, X represents phenyl substituted at the 2-position by halogen.

Preferably, Y represents a substituent selected from hydrogen, halogen —CN, —C₁₋₄alkyl, —C₂₋₄alkenyl, —NR^{a}R^{b}, NO₂, —N(C₁₋₄alkyl)(CHO), —NHCOC₁₋₄alkyl, —NHSO₂R^{c}, C₀₋₄alkylOR^{d}, —C(O)R^{c}, or —C(O)NR^{a}R^{b}. More preferably, Y represents a substituent selected from hydrogen, halogen, —CN, —C₂₋₄alkenyl, —NR^{a}R^{b}, NO₂, —N(C₁₋₄alkyl)(CHO), C₁₋₄alkylOH, —C(O)R^{c}, or —C(O)NR^{a}R^{b}. Even more preferably, Y represents a substituent selected from hydrogen, halogen, —CN, —C₂₋₄alkenyl, —N(C₁₋₄alkyl)(CHO), C₁₋₄alkylOH, —C(O)R^{c}, or —C(O)NR^{a}R^{b}.

Most preferably, Y represents a substituent selected from hydrogen, halogen, —CN, —C(O)R^{c}, or —C(O)NR^{a}R^{b}. Preferably, when X is phenyl, Y is a substituent at the 4-position (i.e. para to the rest o the molecule) on the phenyl ring.

It is to be understood that the present invention covers all combinations of preferred, more preferred, even more preferred and most preferred groups described herein above.

As used herein, the term “alkyl” means both straight and branched chain saturated hydrocarbon groups. Examples of alkyl groups include methyl (—CH₃), ethyl (—C₂H₅), propyl (—C₃H₇) and butyl (—C₄H₉).

As used herein, the term “alkylene” means both straight and branched chain saturated hydrocarbon linker groups. Examples of alkylene groups include methylene (—CH₂—), ethylene (—CH₂CH₂—) and propylene (—CH₂CH₂CH₂—).

As used herein, the term “alkenylene” means both straight and branched chain unsaturated hydrocarbon linker groups, wherein the unsaturation is present only as double bonds. Examples of alkenylene groups includes ethenylene (—CH═CH—) and propenylene (—CH₂—CH═CH—).

As used herein, the term “heterocyclic group” means optionally substituted rings containing one or more heteroatoms selected from: nitrogen, sulphur and oxygen atoms. The heterocycle may be aromatic or non-aromatic, i.e., may be saturated, partially or fully unsaturated. Examples of 5-membered groups include thienyl, furanyl, pyrrolidinyl thiazolyl, oxazolyl and imidazolyl. Examples of 6-membered groups include pyridyl, piperidinyl, pyrimidinyl and morpholinyl. Examples of 7-membered groups include hexamethyleneiminyl. Certain heterocyclic groups, e.g. thienyl, furanyl, thiazolyl, oxazolyl, pyridyl and pyrimidinyl are C-linked to the rest of the molecule. Other heterocyclic groups, e.g pyrrolidinyl, imidazolyl, piperidyl, morpholinyl and hexamethyleneiminyl may be C-linked or N-linked to the rest of the molecule.

As used herein, the term “halogen” means an atom selected from fluorine, chlorine, bromine and iodine.

As used herein, the term “pharmaceutically acceptable” means a compound which is suitable for pharmaceutical use.

As used herein, the term “pharmaceutically acceptable derivative”, means any pharmaceutically acceptable salt, solvate, or prodrug e.g. ester or carbamate, or salt or solvate of such a prodrug, of a compound of formula (I), which upon administration to the recipient is capable of providing (directly or indirectly) a compound of formula (I), or an active metabolite or residue thereof. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and solvates.

Suitable salts according to the invention include those formed with both organic and inorganic acids and bases. Pharmaceutically acceptable acid addition salts include those formed from mineral acids such as: hydrochloric, hydrobromic, sulphuric, phosphoric, acid; and organic acids such as: citric, tartaric, lactic, pyruvic, acetic, trifluoroacetic, succinic, oxalic, formic, fumaric, maleic, oxaloacetic, methanesulphonic, ethanesulphonic, p-toluenesulphonic, benzenesulphonic and isethionic acids. Particularly preferred pharmaceutically acceptable salts include those formed from hydrochloric, trifluoroacetic and formic acids.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of formula (I) are within the scope of the invention.

Salts and solvates of compounds of formula (I) which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of formula (I) and their pharmaceutically acceptable salts and solvates.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference. Esters may be active in their own right and/or be hydrolysable under in vivo conditions in the human body. Suitable pharmaceutically acceptable in vivo hydrolysable ester groups include those which break down readily in the human body to leave the parent acid or its salt.

Preferred compounds of the invention include:
6-Chloro-N-{(3S)-1-[3-fluoro-2′-(methylsulfonyl)-1,1′-biphenyl-4-yl]-2-oxopyrrolidin-3-yl}naphthalene-2-sulfonamide
6-Chloro-N-{(3S)-1-[4-(dimethylamino)phenyl]-2-oxopyrrolidin-3-yl}naphthalene-2-sulfonamide
(E)-2-(5-Chlorothien-2-yl)-N-((3S)-1-{5-[2-(methylsulfonyl)phenyl]pyridin-2-yl}-2-oxopyrrolidin-3-yl)ethenesulfonamide
(E)-2-(5-Chlorothien-2-yl)-N-{(3S)-1-[3-fluoro-2′-(methylsulfonyl)-1,1′-biphenyl-4-yl]-2-oxopyrrolidin-3-yl}ethenesulfonamide
5-Chloro-N-{(3S)-1-[3-fluoro-2′-(methylsulfonyl)-1,1′-biphenyl-4-yl]-2-oxopyrrolidin-3-yl}-1-benzofuran-2-sulfonamide
N-{(3S)-1-[3-Fluoro-2′-(methylsulfonyl)-1,1′-biphenyl-4-yl]-2-oxopyrrolidin-3-yl}isoquinoline-5-sulfonamide
(E)-2-(4-Chlorophenyl)-N-{(3S)-1-[3-fluoro-2′-(methylsulfonyl)-1,1′-biphenyl-4-yl]-2-oxopyrrolidin-3-yl}ethenesulfonamide
5′-Chloro-N-{(3S)-1-[3-fluoro-2′-(methylsulfonyl)-1,1′-biphenyl-4-yl]-2-oxopyrrolidin-3-yl}-2,2′-bithiophene-5-sulfonamide
6-(Dimethylamino)-N-{(3S)-1-[3-fluoro-2′-(methylsulfonyl)-1,1′-biphenyl-4-yl]-2-oxopyrrolidin-3-yl}naphthalene-2-sulfonamide
N-{(3S)-1-[3-Fluoro-2′-(methylsulfonyl)-1,1′-biphenyl-4-yl]-2-oxopyrrolidin-3-yl}quinoline-8-sulfonamide
6-Chloro-N-{(3S)-1-[3-fluoro-2′-(methylsulfonyl)-1,1′-biphenyl-4-yl]-2-oxopyrrolidin-3-yl}-1-benzothiophene-2-sulfonamide
5-Chloro-N-{(3S)-1-[3-fluoro-2′-(methylsulfonyl)-1,1′-biphenyl-4-yl]-2-oxopyrrolidin-3-yl}-1-benzothiophene-2-sulfonamide
6-Chloro-N-[(3S)-1-(4-{2-[(dimethylamino)methyl]-1H-imidazol-1-yl}-2-fluorophenyl)-2-oxopyrrolidin-3-yl]-1-benzothiophene-2-sulfonamide formate salt (1:1)
(1E)-2-(5-Chlorothien-2-yl)-N-[(3S)-1-(4-{2-[(dimethylamino)methyl]-1 H-imidazol-1-yl}-2-fluorophenyl)-2-oxopyrrolidin-3-yl]prop-1-ene-1-sulfonamide formate salt (1:1)
N-{(3S)-1-[2′(Aminosulfonyl)-3-fluoro-1,1′-biphenyl-4-yl]-2-oxopyrrolidin-3-yl}-6-chloro-1-benzothiophene-2-sulfonamide
4′-[(3S)-3-({[(1E)-2-(5-Chlorothien-2-yl)prop-1-enyl]sulfonyl}amino)-2-oxopyrrolidin-1-yl]-3′-fluoro-1,1′-biphenyl-2-sulfonamide
(E)-2-(5-Chlorothien-2-yl)-N-{(3S)-1-[5-(2-nitrophenyl)pyridin-2-yl]-2-oxopyrrolidin-3-yl}ethenesulfonamide
(E)-2-(5-Chlorothien-2-yl)-N-[(3S)-1-(3-fluoro-2′-nitro-1,1 ′-biphenyl-4-yl)-2-oxopyrrolidin-3-yl]ethenesulfonamide
4′-[(3S)-3-({[(E)-2-(5-Chlorothien-2-yl)ethenyl]sulfonyl}amino)-2-oxopyrrolidin-1-yl]-3′-fluoro-N-methyl-1,1′-biphenyl-2-sulfonamide
4′-[(3S)-3-({[(E)-2-(5-Chlorothien-2-yl)ethenyl]sulfonyl}amino)-2-oxopyrrolidin-1-yl]-3′-fluoro-1,1′-biphenyl-2-sulfonamide
(E)-2-(5-Chlorothien-2-yl)-N-[(3S)-1-(5-{2-[(methylsulfonyl)amino]phenyl}pyridin-2-yl)-2-oxopyrrolidin-3-yl]ethenesulfonamide
(E)-N-{(3S)-1-[5-(2-tert-Butylphenyl)pyridin-2-yl]-2-oxopyrrolidin-3-yl}-2-(5-chlorothien-2-yl)ethenesulfonamide
5-Chloro-N-((3S)-1-{5-[2-(methylsulfonyl)phenyl]pyridin-2yl}-2-oxopyrrolidin-3-yl)-1-benzofuran-2-sulfonamide
(E)-2-(5-Chlorothien-2-yl)-N-((3S)-2-oxo-1-{5-[2-(trifluoromethyl)phenyl]pyridin-2-yl}pyrrolidin-3-yl)ethenesulfonamide
2-{6-[(3S)-3-({[(E)-2-(5-Chlorothien-2-yl)ethenyl]sulfonyl}amino)-2-oxopyrrolidin-1-yl]pyridin-3-yl}-N,N-dimethylbenzamide
(E)-2-(5-Chlorothien-2-yl)-N-{(3S)-1-[5-(2-cyanophenyl)pyridin-2-yl]-2-oxopyrrolidin-3-yl}ethenesulfonamide
2-{6-[(3S)-3-({[(E)-2-(5-Chlorothien-2-yl)ethenyl]sulfonyl}amino)-2-oxopyrrolidin-1-yl]pyridin-3-yl}benzenesulfonamide
2-{6-[(3S)-3-({[(E)-2-(5-Chlorothien-2-yl)ethenyl]sulfonyl}amino)-2-oxopyrrolidin-1-yl]pyridin-3-yl}-N,N-dimethylbenzenesulfonamide
2-{6-[(3S)-3-({[(E)-2-(5-Chlorothien-2-yl)ethenyl]sulfonyl}amino)-2-oxopyrrolidin-1-yl]pyridin-3-yl}-N-methylbenzenesulfonamide
(E)-2-(5-Chlorothien-2-yl)-N-[(3S)-1-(5-{2-[methyl(methylsulfonyl)amino]phenyl}pyridin-2-yl)-2-oxopyrrolidin-3-yl]ethenesulfonamide
(E)-2-(5-Chlorothien-2-yl)-N-{(3S)-1-[5-(2-isopropoxyphenyl)pyridin-2-yl]-2-oxopyrrolidin-3-yl}ethenesulfonamide
6-Chloro-N-[(3S)-2-oxo-1-(5-phenylpyridin-2-yl)pyrrolidin-3-yl]naphthalene-2-sulfonamide
5-Chloro-N-((3S)-1-{5-[2-(methylsulfonyl)phenyl]pyridin-2-yl}-2-oxopyrrolidin-3-yl)thieno[2,3-b]pyridine-2-sulfonamide
4-Cyano-N-{(3S)-1-[3-fluoro-2′-(methylsulfonyl)-1,1′-biphenyl-4-yl]-2-oxopyrrolidin-3-yl)benzenesulfonamide
3-Cyano-N-{(3S)-1-[3-fluoro-2′-(methylsulfonyl)-1,1′-biphenyl-4-yl]-2-oxopyrrolidin-3-yl}benzenesulfonamide
6-Chloro-N-{(3S)-1-[3-fluoro-2′-(methylsulfonyl)-1,1′-biphenyl-4-yl]-2-oxopyrrolidin-3-yl}-1-benzofuran-2-sulfonamide
6-Chloro-N-{(3S)-1-[3-fluoro-2′-(methylsulfonyl)-1,1′-biphenyl-4-yl]-2-oxopyrrolidin-3-yl}thieno[3,2-b]pyridine-2-sulfonamide
5-Chloro-N-{(3S)-1-[3-fluoro-2′-(methylsulfonyl)-1,1′-biphenyl-4-yl]-2-oxopyrrolidin-3-yl}thieno[3,2-b]pyridine-2-sulfonamide
(1E)-2-(5-Chlorothien-2-yl)-N-{(3S)-1-[3-fluoro-2′-(methylsulfonyl)-1,1′-biphenyl-4-yl]-2-oxopyrrolidin-3-yl}prop-1-ene-1-sulfonamide
tert-Butyl [{[(E)2-(5-Chlorothien-2-yl)ethenyl]sulfonyl}((3S)-1-{5-[2-(methylsulfonyl)phenyl]pyridin-2-yl}-2-oxopyrrolidin-3-yl)amino]acetate
[{[(E)-2-(5-Chlorothien-2-yl)ethenyl]sulfonyl}((3S)-1-{5-[2-(methylsulfonyl)phenyl]pyridin-2-yl}-2-oxopyrrolidin-3-yl)amino]acetic acid
(E)-2-(5-Chlorothien-2-yl)-N-((3S)-1-{5-[2-(methylsulfonyl)phenyl]pyridin-2-yl}-2-oxopyrrolidin-3-yl)-N-(2-morpholin-4-ylethyl)ethenesulfonamide formate salt (1:1)
2-[{[(E)-2-(5-Chlorothien-2-yl)ethenyl]sulfonyl}((3S)-1-{5-[2-(methylsulfonyl)phenyl]pyridin-2-yl}-2-oxopyrrolidin-3-yl)amino]acetamide
tert-Butyl [(E)-2-(5-chlorothien-2-yl)ethenyl]sulfonyl{(3S)-1-[3-fluoro-2′-(methylsulfonyl)-1,1′-biphenyl-4-yl]-2-oxopyrrolidin-3-yl}carbamate
(E)-2-(5-Chlorothien-2-yl)-N-{(3S)-1-[3-fluoro-2′-(methylsulfonyl)-1,1′-biphenyl-4-yl]-2-oxopyrrolidin-3-yl}-N-(2-morpholin-4-ylethyl)ethenesulfonamide
2-({[(E)-2-(5-Chlorothien-2-yl)ethenyl]sulfonyl}{(3S)-1-[3-fluoro-2′-(methylsulfonyl)-1,1′-biphenyl-4-yl]-2-oxopyrrolidin-3-yl}amino)acetamide
tert-Butyl ({[(E)-2-(5-chlorothien-2-yl)ethenyl]sulfonyl}{(3S)-1-[3-fluoro-2′-(methylsulfonyl)-1,1′-biphenyl-4-yl]-2-oxopyrrolidin-3-yl}amino)acetate
{[(E)-2-(5-Chlorothien-2-yl)ethenyl]sulfonyl}((3S)-1-{5-[2-(methylsulfonyl)phenyl]pyridin-2-yl}-2-oxopyrrolidin-3-yl)amino]acetic acid
({[(E)-2-(5-Chlorothien-2-yl)ethenyl]sulfonyl}{(3S)-1-[3-fluoro-2′-(methylsulfonyl)-1,1′-biphenyl-4-yl]-2-oxopyrrolidin-3-yl}amino)acetic acid
4′-[(S)-3-(6-Chloro-naphthalene-2-sulphonylamino)-2-oxo-pyrrolidin-1-yl]-3′-fluoro-biphenyl-3-carboxylic acid amide
6-Chloro-naphthalene-2-sulphonic acid [(S)-1-[5-(2-methylsulphanylphenyl)-thiazol-2-yl]-2-oxo-pyrrolidin-3-yl]amide
6-Chloro-naphthalene-2-sulphonic acid [(S)-1-[5-(2-methanesulphonylphenyl)-thiazol-2-yl]-2-oxo-pyrrolidin-3-yl]amide
3-(Aminomethyl)-N-{(3S)-1-[3-fluoro-2′-(methylsulfonyl)-1,1′-biphenyl-4-yl]-2-oxopyrrolidin-3-yl}benzenesulfonamide
4-(Aminomethyl)-N-{(3S)-1-[3-fluoro-2′-(methylsulfonyl)-1,1′-biphenyl-4-yl]-2-oxopyrrolidin-3-yl}benzenesulfonamide
6-Chloro-N-[(3S)-1-(2-fluoro-4-pyridin-4-ylphenyl)-2-oxopyrrolidin-3-yl]naphthalene-2-sulfonamide
6-Chloro-N-{(3S)-1-[4-(2,4-dimethoxypyrimidin-5-yl)-2-fluorophenyl]-2-oxopyrrolidin-3-yl}naphthalene-2-sulfonamide
6-Chloro-N-[(3S)-1-(2-fluoro-4-pyridin-3-ylphenyl)-2-oxopyrrolidin-3-yl]naphthalene-2-sulfonamide
6-Chloro-N-{(3S)-1-[2-fluoro-4-(6-methoxypyridin-3-yl)phenyl]-2-oxopyrrolidin-3-yl}naphthalene-2-sulfonamide
6-Chloro-N-{(3S)-1-[2-fluoro-4-(4-propylpyridin-3-yl)phenyl]-2-oxopyrrolidin-3-yl}naphthalene-2-sulfonamide
6-Chloro-N-((3S)-1-{2-fluoro-4-[6-(methylthio)pyridin-3-yl]phenyl}-2-oxopyrrolidin-3-yl)naphthalene-2-sulfonamide
N-{(3S)-1-[4-(5-bromopyridin-3-yl)-2-fluorophenyl]-2-oxopyrrolidin-3-yl}-6-chloronaphthalene-2-sulfonamide
6-Chloro-N-{(3S)-1-[2-fluoro-4-(4-methoxypyridin-3-yl)phenyl]-2-oxopyrrolidin-3-yl}naphthalene-2-sulfonamide
6-Chloro-N-[(3S)-1-(2-fluoro-4-pyrimidin-5-ylphenyl)-2-oxopyrrolidin-3-yl]naphthalene-2-sulfonamide
N-{(3S)-1-[3′-(aminomethyl)-3-fluoro-1,1′-biphenyl-4-yl]-2-oxopyrrolidin-3-yl}-6-chloronaphthalene-2-sulfonamide
6-Chloro-N-{(3S)-1-[2-fluoro-4-(3-furyl)phenyl]-2-oxopyrrolidin-3-yl}naphthalene-2-sulfonamide
6-Chloro-N-{(3S)-1-[2-fluoro-4-(4-methylthien-2-yl)phenyl]-2-oxopyrrolidin-3-yl}naphthalene-2-sulfonamide
6-Chloro-N-[(3S)-1-(2-fluoro-4-thien-3-ylphenyl)-2-oxopyrrolidin-3-yl]naphthalene-2-sulfonamide
6-Chloro-N-{(3S)-1-[2-fluoro-4-(5-methylthien-2-yl)phenyl]-2-oxopyrrolidin-3-yl}naphthalene-2-sulfonamide
6-Chloro-N-{(3S)-1-[2-fluoro-4-(4-methylthien-3-yl)phenyl]-2-oxopyrrolidin-3-yl}naphthalene-2-sulfonamide
6-Chloro-N-{(3S)-1-[2-fluoro-4-(3-formylthien-2-yl)phenyl]-2-oxopyrrolidin-3-yl}naphthalene-2-sulfonamide
6-Chloro-N-{(3S)-1-[4-(5-chlorothien-2-yl)-2-fluorophenyl]-2-oxopyrrolidin-3-yl}naphthalene-2-sulfonamide
6-Chloro-N-{(3S)-1-[4-(3,5-dimethylisoxazol-4-yl)-2-fluorophenyl]-2-oxopyrrolidin-3-yl}naphthalene-2-sulfonamide
6-Chloro-N-{(3S)-1-[2-fluoro-4-(5-methyl-2-furyl)phenyl]-2-oxopyrrolidin-3-yl}naphthalene-2-sulfonamide
6-Chloro-N-[(3S)-1-(3-fluoro-1,1′-biphenyl-4-yl)-2-oxopyrrolidin-3-yl]naphthalene-2-sulfonamide
(E)-2-(5-Chlorothien-2-yl)-N-[(3S)-1-(4-{2-[(dimethylamino)methyl]-1H-imidazol-1-yl}-2-fluorophenyl)-2-oxopyrrolidin-3-yl]ethenesulfonamide bis(trifluoroacetate)
6-Chloro-N-{(3S)-1-[2-fluoro-4-(1-oxidopyridin-4-yl)phenyl]-2-oxopyrrolidin-3-yl}naphthalene-2-sulfonamide
6-Chloro-N-{(3S)-1-[2-fluoro-4-(1-methyl-1H-imidazol-2-yl)phenyl]-2-oxopyrrolidin-3-yl}naphthalene-2-sulfonamide
6-Chloro-N-{(3S)-1-[4-(2-chloropyridin-3-yl)-2-fluorophenyl]-2-oxopyrrolidin-3-yl}naphthalene-2-sulfonamide
6-Chloro-N-{(3S)-1-[4-(2-cyanopyridin-3-yl)-2-fluorophenyl]-2-oxopyrrolidin-3-yl}naphthalene-2-sulfonamide
(E)-N-{(3S)-1-[4-(3-Chloropyridin-4-yl)-2-fluorophenyl]-2-oxopyrrolidin-3-yl}-2-(5-chlorothien-2-yl)ethenesulfonamide
6-Chloro-N-[(3S)-1-(2-fluoro-4-pyrimidin-2-ylphenyl)-2-oxopyrrolidin-3-yl]naphthalene-2-sulfonamide
6-Chloro-N-{(3S)-1-[4-(3-chloropyridin-2-yl)-2-fluorophenyl]-2-oxopyrrolidin-3-yl}naphthalene-2-sulfonamide
6-chloro-N-{(3S)-1-[4-(3-chloropyridin-4-yl)-2-fluorophenyl]-2-oxopyrrolidin-3-yl}naphthalene-2-sulfonamide
6-Chloro-N-{(3S)-1-[2-fluoro-4-(1-methyl-1H-imidazol-4-yl)phenyl]-2-oxopyrrolidin-3-yl}naphthalene-2-sulfonamide formate
6-Chloro-N-{(3S)-1-[2-fluoro-4-(1-methyl-1H-imidazol-5-yl)phenyl]-2-oxopyrrolidin-3-yl}naphthalene-2-sulfonamide
2-(5-Chlorothien-2-yl)-N-{(3S)-1-[3-fluoro-2′-(methylsulfonyl)-1,1′-biphenyl-4-yl]-2-oxopyrrolidin-3-yl}-1,3-thiazole-5-sulfonamide
5-Chloro-N-{(3S)-1-[3-fluoro-2′-(methylsulfonyl)-1,1′-biphenyl-4-yl]-2-oxopyrrolidin-3-yl}thieno[3,2-b]thiophene-2-sulfonamide
2-Chloro-N-{(3S)-1-[3-fluoro-2′-(methylsulfonyl)-1,1′-biphenyl-4-yl]-2-oxopyrrolidin-3-yl}thieno[3,2-b]thiophene-3-sulfonamide
6-Chloro-N-[(3S)-1-(2-fluoro-4-iodophenyl)-2-oxopyrrolidin-3-yl]naphthalene-2-sulfonamide
(E)-2-(5-Chlorothien-2-yl)-N-[(3S)-1-(5-iodopyridin-2-yl)-2-oxopyrrolidin-3-yl}ethenesulfonamide
6-Chloro-N-[(3S)-1-(2-fluoro-4-iodophenyl)-2-oxopyrrolidinyl]-1benzothiophene-2-sulfonamide X
5′-Chloro-N-[(3S)-1-(2-fluoro-4-iodophenyl)-2-oxopyrrolidin-3-yl]-2,2′-bithiophene-5-sulfonamide
2-(5-Chloro-2-thienyl)-N-[(3S)-1-(2-fluoro-4-iodophenyl)-2-oxopyrrolidinyl]ethanesulfonamide
6-Chloro-N-[(3R)-1-(2-fluoro-4-nitrophenyl)-2-oxopyrrolidinyl]-1-benzothiophene-2-sulfonamide
(E)-2-(5-Chloro-2-thienyl)-N-[(3S)-1-(2-fluoro-4-nitrophenyl)-2-oxopyrrolidinyl]ethenesulfonamide
5′-Chloro-N-[(3S)-1-(2-fluoro-4-nitrophenyl)-2-oxopyrrolidin-3-yl]-2,2′-bithiophene-5-sulfonamide
6-Chloro-N-[(3S)-1-(4-cyano-2-fluorophenyl)-2-oxopyrrolidin-3-yl]-1-benzothiophene-2-sulfonamide
(E)-2-(5-Chlorothien-2-yl)-N-[(3S)-1-(4-cyano-2-fluorophenyl)-2-oxopyrrolidin-3-yl]ethenesulfonamide
2-(5-Chlorothien-2-yl)-N-[(3 S)-1-(4-cyano-2-fluorophenyl)-2-oxopyrrolidin-3-yl]ethanesulfonamide
(E)-2-(5-Chloro-2-thienyl)-N-[(3S)-1-(2-fluoro-4-isopropenylphenyl)-2-oxopyrrolidinyl]ethenesulfonamide
6-Chloro-N-[(3S)-1-(2-fluorophenyl)-2-oxopyrrolidin-3-yl]naphthalene-2-sulfonamide
N-[(3S)-1-(4-Bromo-2-fluorophenyl)-2-oxopyrrolidinyl]-6-chloro-2-naphthalenesulfonamide
6-Chloro-N-{(3S)-1-[3-fluoro-4-(4-morpholinyl)phenyl]-2-oxopyrrolidinyl}-2-naphthalenesulfonamide
4-[(3S)-3-({[(E)-2-(5-Chlorothien-2-yl)ethenyl]sulfonyl}amino)-2-oxopyrrolidin-1-yl]-3-fluoro-N,N-dimethylbenzamide
(E)-2-(5-chloro-2-thienyl)-N-{(3S)-1-[2-fluoro-4-(1-pyrrolidinylcarbonyl)phenyl]-2-oxopyrrolidinyl}ethenesulfonamide
6-[(3S)-3-({[(E)-2-(5-Chlorothien-2-yl)ethenyl]sulfonyl}amino)-2-oxopyrrolidin-1-yl]nicotinamide
4-[(3S)-3-({[(E)-2-(5-Chlorothien-2-yl)ethenyl]sulfonyl}amino)-2-oxopyrrolidin-1-yl]-3-fluorobenzamide
4-[(3S)-3-({[(E)-2-(5-Chlorothien-2-yl)ethenyl]sulfonyl}amino)-2-oxopyrrolidin-1-yl]-3-fluoro-N-methylbenzamide
4-((3S)-3-{[(6-Chloro-1-benzothien-2-yl)sulfonyl]amino}-2-oxopyrrolidin-1-yl)-3-fluoro-N,N-dimethylbenzamide
4-[(3S)-3-({[(1E)-2-(5-Chlorothien-2-yl)prop-1-enyl]sulfonyl}amino)-2-oxopyrrolidin-1-yl]-3-fluoro-N,N-dimethylbenzamide
4-((3S)-3-{[(6-Chloro-1-benzothien-2-yl)sulfonyl]amino}-2-oxopyrrolidin-1-yl)-3-fluoro-N-isopropyl-N-methylbenzamide
(E)-N-[(3S)-1-(4-acetyl-2-fluorophenyl)-2-oxopyrrolidin-3-yl]-2-(5-chlorothien-2-yl)ethenesulfonamide
(E)-N-[(3S)-1-(5-Acetylpyridin-2-yl)-2-oxopyrrolidin-3-yl]-2-(5-chlorothien-2-yl)ethenesulfonamide
N-{4-[(3S)-3-({[(E)-2-(5-Chloro-2-thienyl)ethenyl]sulfonyl}amino)-2-oxopyrrolidinyl]-3-fluorophenyl}acetamide
N-{4-[(3S)-3-({[(E)-2-(5-Chloro-2-thienyl)ethenyl]sulfonyl}amino)-2-oxopyrrolidinyl]-3-fluorophenyl}propanamide
N-{4-[(3S)-3-({[(E)-2-(5-Chlorothien-2-yl)ethenyl]sulfonyl}amino)-2-oxopyrrolidin-1-yl]-3-fluorophenyl}-2-methylpropanamide
N-[4-((3S)-3-{[(6-Chloro-1-benzothien-2-yl)sulfonyl]amino}-2-oxopyrrolidinyl)-3-fluorophenyl]acetamide
N-[4-((3S)-3-{[(6-Chloro-1-benzothien-2-yl)sulfonyl]amino}-2-oxopyrrolidinyl)-3-fluorophenyl]propanamide
N-[4-((3S)-3-{[(6-Chloro-1-benzothien-2-yl)sulfonyl]amino}-2-oxopyrrolidinyl)-3-fluorophenyl]-2-methylpropanamide
(E)-2-(5-chlorothien-2-yl)-N-((3S)-1-{2-fluoro-4-[formyl(isopropyl)amino]phenyl}-2-oxopyrrolidin-3-yl)ethenesulfonamide
6-Chloro-N-((3S)-1-{2-fluoro-4-formyl(isopropyl)amino]phenyl}-2-oxopyrrolidin-3-yl)-1-benzothiophene-2-sulfonamide
6-Chloro-N-{(3S)-1-[2-fluoro-4-(1H-imidazol-yl)phenyl]-2-oxopyrrolidin-3-yl}naphthalene-2-sulfonamide
6-Chloro-N-[(3S)-1-(2,4-dichlorophenyl)-2-oxopyrrolidinyl]-2-naphthalenesulfonamide
N-[(3S)-1-(4-tert-Butyl-1,3-thiazol-2-yl)-2-oxopyrrolidinyl]-6-chloro-2-naphthalenesulfonamide
N-[(3S)-1-(4-tert-butylphenyl)-2-oxopyrrolidinyl]-6-chloro-2-naphthalenesulfonamide
(1E)-2-(5-chlorothien-2-yl)-N-[(3S)-2-oxo-1-pyrazin-2-ylpyrrolidin-3-yl]prop-1-ene-1-sulfonamide
6-Chloro-N-[(3S)-2-oxo-1-(1,3-thiazol-2-yl)pyrrolidinyl]-2-naphthalenesulfonamide
6-Chloro-N-{(3S)-1-[2-fluoro-4-(4-methyl-1H-imidazol-1-yl)phenyl]-2-oxopyrrolidinyl}-2-naphthalenesulfonamide
6-Chloro-N-{(3S)-1-[2-fluoro-4-(1H-pyrazol-1-yl)phenyl]-2-oxopyrrolidinyl}-2-naphthalenesulfonamide
N-[(3S)-1-(5-Bromo-1,3-thiazol-2-yl)-2-oxopyrrolidinyl]-2-(5-chloro-2-thienyl)ethanesulfonamide
6-Chloro-N-[(3S)-1-(pyrazin-2-yl)-2-oxopyrrolidin-3-yl]-1-benzothiphene-2-sulfonamide
2-(5-Chlorothien-2-yl)-N-[(3S)-1-(5-iodopyridin-2-yl)-2-oxopyrrolidin-3-yl]ethane-1-sulfonamide
4-[(3S)-3-((2-Amino-2-oxoethyl){[(E)-2-(5-chlorothien-2-yl)ethenyl]sulfonyl}amino)-2-oxopyrrolidin-1-yl]-3-fluorobenzamide
4-[(3S)-3-((2-Amino-2-oxoethyl){[(E)-2-(5-chlorothien-2-yl) ethenyl]sulfonyl}amino)-2-oxopyrrolidin-1-yl]-3-fluoro-N,N-dimethylbenzamide
(E)-2-(5-Chlorothien-2-yl)-N-{(3S)-1-[2-fluoro-4-(1-hydroxyethyl)phenyl]-2-oxopyrrolidin-3-yl}ethenesulfonamide
(1E)-2-(5-chlorothien-2-yl)-N-[(3S-1-(5-iodopyridin-2-yl)-2-oxopyrrolidin-3-yl]prop-1-ene-1-sulfonamide
(1E)-2-(5-chlorothien-2-yl)-N-((3S)-1-{2-fluoro-4-[(methylsulfonyl)amino]phenyl}-2-oxopyrrolidin-3-yl)prop-1-ene-1-sulfonamide
(E)-N-[(3S)-1-(4-acetylphenyl)-2-oxopyrrolidin-3-yl]-2-(5-chlorothien-2-yl)ethenesulfonamide
2-({(3S)-1-[2′-(Aminosulfonyl)-3-fluoro-1,1′-biphenyl-4-yl]-2-oxopyrrolidin-3-yl}{[(1E)-2-(5-chlorothien-2-yl)prop-1-enyl]sulfonyl}amino)acetamide
2-({(3S)-1-[2′-(Aminosulfonyl)-3-fluoro-1,1′-biphenyl-4-yl]-2-oxopyrrolidin-3-yl}{[(1Z)-2-(5-chlorothien-2-yl)prop-1-enyl]sulfonyl}amino)acetamide
2-{(6-Chloro-benzo[b]thiophene-2-sulphonyl)-[(S)-1-(3-fluoro-2′-sulfamoyl-biphenyl-4-yl)-2-oxo-pyrrolidin-3-yl]-amino}-acetamide formate
2-(5-Chlorothien-2-yl)-N-[(3S)-1-(4-{2-[(dimethylamino)methyl]-1H-imidazol-1-yl}-2-fluorophenyl)-2-oxopyrrolidin-3-yl]ethanesulfonamide
2-Amino-N-[(1-{4-[(3S)-3-({[(1E)-2-(5-chlorothien-2-yl)prop-1-enyl]sulfonyl}amino)-2-oxopyrrolidin-1-yl]-3-fluorophenyl}-1H-imidazol-2-yl)methyl]-N,N-dimethyl-2-oxoethanaminium formate
2-Amino-N-[1-{4-[(3S)-3-({[2-(5-chlorothien-2-yl)ethyl]sulfonyl}amino)-2-oxopyrrolidin-1-yl]-3-fluorophenyl}-1H-imidazol-2-yl)methyl]-N,N-dimethyl-2-oxoethanaminium formate
2-Amino-N-({1-[4-((3S)-3-{[(6-chloro-1-benzothien-2-yl)sulfonyl]amino}-2-oxopyrrolidin-1yl)-3-fluorophenyl]-1H-imidazol-2-yl}methyl)-N,N-dimethyl-2-oxoethanaminium formate

Compounds of the invention may show advantageous properties, they may be more efficacious, show greater selectivity, have fewer side effects, have a longer duration of action, be more bioavailable by the preferred route, or have other more desirable properties than similar known compounds.

The compounds of formula (I) are Factor Xa inhibitors and as such are useful in the treatment of clinical conditions susceptible to amelioration by administration of a Factor Xa inhibitor. Such conditions include acute vascular diseases such as coronary thrombosis (for example myocardial infarction and unstable angina), thromboembolism, acute vessel closure associated with thrombolytic therapy and percutaneous transluminal coronary angioplasty (PTCA), transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke; in oedema and PAF mediated inflammatory diseases such as adult respiratory shock syndrome, septic shock and reperfusion damage; the treatment of pulmonary fibrosis; the treatment of tumour metastasis; neurogenerative disease such as Parkinson's and Alzheimer's diseases; viral infection; Kasabach Merritt Syndrome; Haemolytic uremic syndrome; arthritis; osteoporosis; as anti-coagulants for extracorporeal blood in for example, dialysis, blood filtration, bypass, and blood product storage; and in the coating of invasive devices such as prostheses, artificial valves and catheters in reducing the risk of thrombus formation.

Accordingly, one aspect of the present invention provides a compound of formula (I) or a pharmaceutically acceptable derivative thereof for use in medical therapy, particularly for use in the amelioration of a clinical condition in a mammal, including a human, for which a Factor Xa inhibitor is indicated.

In another aspect, the invention provides a method for the treatment and/or prophylaxis of a mammal, including a human, suffering from a condition susceptible to amelioration by a Factor Xa inhibitor which method comprises administering to the subject an effective amount of a compound of formula (I) or a pharmaceutically acceptable derivative thereof.

In another aspect, the present invention provides the use of a compound of formula (I) or a pharmaceutically acceptable derivative thereof, for the manufacture of a medicament for the treatment and/or prophylaxis of a condition susceptible to amelioration by a Factor Xa inhibitor.

Preferably, the condition susceptible to amelioration by a Factor Xa inhibitor is selected from treatment of acute vascular diseases such as coronary thrombosis (for example myocardial infarction and unstable angina), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke.

More preferably, the condition susceptible to amelioration by a Factor Xa inhibitor is selected from coronary thrombosis (for example myocardial infarction and unstable angina), pulmonary embolism, deep vein thrombosis and the prevention of thromboembolic events associated with atrial fibrillation, e.g. stroke;

It will be appreciated that reference to treatment includes acute treatment or prophylaxis as well as the alleviation of established symptoms.

While it is possible that, for use in therapy, a compound of the present invention may be administered as the raw chemical, it is preferable to present the active ingredient as a pharmaceutical formulation.

In a further aspect, the invention provides a pharmaceutical composition comprising at least one compound of formula (I) or a pharmaceutically acceptable derivative thereof in association with a pharmaceutically acceptable carrier and/or excipient. The carrier and/or excipient must be “acceptable” in the sense of being compatible with the other ingredients of the formulation and not deletrious to the receipient thereof.

Accordingly, the present invention further provides a pharmaceutical formulation comprising at least one compound of formula (I) or a pharmaceutically acceptable derivative thereof, in association with a pharmaceutically acceptable carrier and/or excipient. The carrier and/or excipient must be “acceptable” in the sense of being compatible with the other ingredients of the formulation and not deletrious to the receipient thereof.

In another aspect, the invention provides a pharmaceutical composition comprising, as active ingredient, at least one compound of formula (I) or a pharmaceutically acceptable derivative thereof in association with a pharmaceutically acceptable carrier and/or excipient for use in therapy, and in particular in the treatment of human or animal subjects suffering from a condition susceptible to amelioration by a Factor Xa inhibitor.

There is further provided by the present invention a process of preparing a pharmaceutical composition, which process comprises mixing at least one compound of formula (I) or a pharmaceutically acceptable derivative thereof, together with a pharmaceutically acceptable carrier and/or excipient.

The compounds for use according to the present invention may be formulated for oral, buccal, parenteral, topical, rectal or transdermal administration or in a form suitable for administration by inhalation or insufflation (either through the mouth or the nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions or they may be presented as a dry product for constitution with water or other suitable vehicles before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the compositions may take the form of tablets or lozenges formulated in a conventional manner.

The compounds according to the present invention may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds according to the present invention may be formulated for topical administration by insufflation and inhalation. Examples of types of preparation for topical administration include sprays and aerosols for use in an inhaler or insufflator.

Powders for external application may be formed with the aid of any suitable powder base, for example, lactose, talc or starch. Spray compositions may be formulated as aqueous solutions or suspensions or as aerosols delivered from pressurised packs, such as metered dose inhalers, with the use of a suitable propellant.

The compounds according to the present invention may also be formulated in rectal compositions such as suppositories or retention enemas, e.g. containing conventional suppository bases such as cocoa butter or other glycerides.

In addition to the formulations described previously, the compounds may also be formulated as a depot preparation. Such long acting formulations may be administered by implantation (for example subcutaneously, transcutaneously or intramuscularly) or by intramuscular injection. Thus, for example, the compounds according to the present invention may be formulated with suitable polymeric or hydrophobic materials (for example as an emulsion in an acceptable oil) or ion exchange resins or as sparingly soluble derivatives, for example, as a sparingly soluble salt.

A proposed dose of the compounds according to the present invention for administration to a human (of approximately 70 kg body weight) is 0.1 mg to 1 g, preferably to 1 mg to 500 mg of the active ingredient per unit dose, expressed as the weight of free base. The unit dose may be administered, for example, 1 to 4 times per day. The dose will depend on the route of administration. It will be appreciated that it may be necessary to make routine variations to the dosage depending on the age and weight of the patient as well as the severity of the condition to be treated. The dosage will also depend on the route of administration. The precise dose and route of administration will ultimately be at the discretion of the attendant physician or veterinarian.

The compounds of formula (I) may also be used in combination with other therapeutic agents. The invention thus provides, in a further aspect, a combination comprising a compound of formula (I) or a pharmaceutically acceptable derivative thereof together with a further therapeutic agent.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with other antithrombotic drugs such as thrombin inhibitors, thromboxane receptor antagonists, prostacyclin mimetics, phosphodiesterase inhibitors, fibrinogen antagonists, thrombolytic drugs such as tissue plaminogen activator and streptokinase, non-steroidal anti-inflammatory drugs such as aspirin, and the like.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the Factor Xa inhibitor or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compounds of formula (I) and pharmaceutically acceptable derivatives thereof may be prepared by the processes described hereinafter, said processes constituting a further aspect of the invention. In the following description, the groups are as defined above for compounds of formula (I) unless otherwise stated.

According to a further aspect of the present invention, there is provided a process (A) for preparing a compound of formula (I) which comprises of reacting a compound of formula (II) with a compound of formula (III) wherein V is a reactive group, such as a halide, preferably chloride. The reaction is conveniently carried out in the presence of a base, e.g. pyridine, and in a suitable solvent, e.g. DCM, suitably at room temperature.

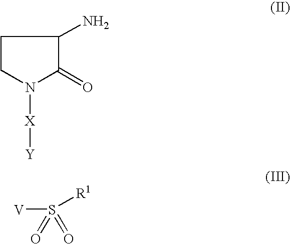

Compounds of formula (III) may be prepared by methods known in the literature or processes known to those skilled in the art¹.

Compounds of formula (II) may be prepared from compounds of formula (IV): 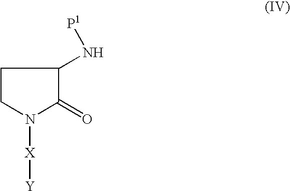
wherein P¹ is a suitable amine protecting group, e.g. Boc (t-Butyloxycarbonyl), by removal of the protecting group under standard conditions. For example, when P¹ represents Boc, removal of the protecting group may be effected under acidic conditions, using for example TFA (trifluoroacetic acid) in a solvent such as DCM or hydrogen chloride in dioxan, suitably at room temperature.

Compounds of formula (IV) may be prepared from compounds of formula (V): 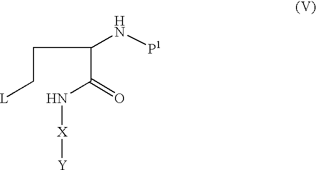
by cyclisation where L represents a leaving group. For example when L is a hydroxyl group, the ring closure may be performed by treatment with an aryl or alkyl phosphine, e.g., tri-n-butylphosphine, and a dialkyl azodicarboxylate, e.g., diisopropyl azodicarboxylate, in a suitable solvent, e.g. THF (tetrahydrofuran).

It will be appreciated by persons skilled in the art that compounds of formula (V) may be prepared by interconversion, utilising other compounds of formula (V) which are optionally protected by standard protecting groups, as precursors. For instance, compounds of formula (V) where L is OH, may be converted into compounds of formula (V) possessing alternative substituents at L, e.g. halogen, ⁺SMeRW⁻ or OSO₂R, by methods well known in the art (see for example Smith, M. B. and March, J., Advanced Organic Chemistry, 5^{th} Edition 2001, John Wiley & Sons). Generally R will represent alkyl or aralkyl and W will represent halide, especially iodide or sulphate. In such cases the ring closure may be performed by treatment with a base in a suitable solvent, e.g. MeCN.

Compounds of formula (V), where L is a hydroxyl group, may be prepared by reacting a compound of formula (VI) with a compound of formula (VII): 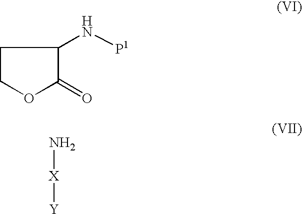
wherein P¹ is a suitable protecting group as described above. The reaction is conveniently carried out by addition of a Lewis acid, e.g. trimethylaluminium, to compounds of formula (VII) in a suitable solvent e.g. DCM, under an inert atmosphere, e.g., nitrogen, suitably at room temperature followed by addition of a compounds of formula (VI) in a compatible solvent e.g., DCM.

Compounds of formula (VI) may be prepared from compounds of formula (VIII) where HA is a suitable salt, e.g., hydrochloride, using methods well known to those skilled in the art. See, for example, “Protective groups in organic synthesis” by T. W. Greene and P. G. M. Wuts (John Wiley & sons 1991) or “Protecting Groups” by P. J. Kocienski (Georg Thieme Verlag 1994).

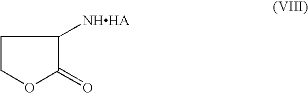

There is provided a further process (B) for preparing compounds of formula (IV).

According to process (B), compounds of formula (IV) may be prepared by metal-catalysed coupling of a compound of formula (IX) with a compound of formula (X) where C¹ and C² are suitable coupling groups, e.g., when bonded to a ring carbon atom C¹ and C² can be boronate [B(OH)₂], halide preferably iodide (I), trifluoromethanesulfonate (OTf) or stannane such as trialkyltin, and P¹ is as defined above. C² can also be hydrogen when directly bonded to a heteroatom of Y. A suitable metal catalyst includes palladium(0) or a salt thereof in the presence of a ligand, e.g., triphenylphosphine and a base, e.g., sodium carbonate, and optionally with a suitable co-solvent, e.g., water, suitably at temperature range from room temperature to 150° C. For example, when C¹ is B(OH)₂ and C² is a bromide, coupling of compounds of formula (IX) with compounds of formula (X) can be effected with tetrakis(triphenylphosphine)palladium(0) in the presence of sodium carbonate in aqueous tetrahydrofuran at 75° C.

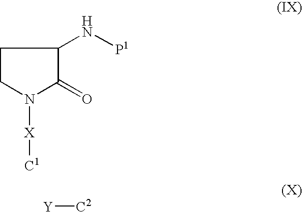

It will be appreciated by persons skilled in the art that certain combinations of coupling groups C¹ and C² in compounds of formula (IX) and (X) and metal catalysts are preferred. Examples of these can be found in Smith, M. B. and March, J., Advanced Organic Chemistry, 5^{th} Edition 2001, John Wiley & Sons. Furthermore, persons skilled in the art will also appreciated that coupling groups, C¹ and C², in compounds of formula (IX) and (X) may be interconverted using known methods.

Alternatively, where Y—C² represents a group NHR^{a}R^{b}, i.e., when C² is a hydrogen directly bonded to a heteroatom of Y, compounds of formula (IV) may be prepared by metal-catalysed coupling of a compound of formula (IX) with a compound of formula (X) where C¹ is a suitable coupling group, e.g., boronate [B(OH)₂], halide preferably iodide (I), and P¹ is as defined above. Suitable metal catalysts include palladium(0) or a salt thereof in the presence of a ligand, e.g., tri-o-tolylphosphine or a copper salt e.g., copper (I) iodide and a base, e.g., sodium tert-butoxide or potassium carbonate, and optionally with a suitable co-solvent, e.g., triethylamine, suitably at temperature range from room temperature to 150° C. For example, when C¹ is iodide, coupling of compounds of formula (IX) with compounds of formula (X) can be effected with copper (I) iodide in the presence of potassium carbonate in dimethylsulfoxide at 123° C. or tris(dibenzylideneacetone)dipalladium (0) and tri-o-tolylphosphine in the presence of sodium tert-butoxide in dioxan at 100° C.

Accordingly, compounds of formula (IX) may be prepared from compounds of formula (XI) where P¹, L and C¹ are defined as above: 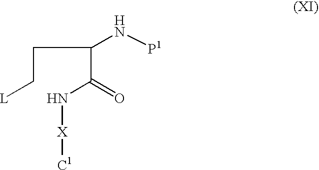
by cyclisation where L represents a leaving group. For example when L is a hydroxyl group, the ring closure may be performed by treatment with an aryl or alkyl phosphine, e.g., tri-n-butylphosphine, and a dialkyl azodicarboxylate, e.g., diisopropyl azodicarboxylate, in a suitable solvent, e.g. THF (tetrahydrofuran).

Compounds of formula (XI), where L is a hydroxyl group, may be prepared by reacting compounds of formula (VI) with NH₂—XC¹. The reaction is conveniently carried out by addition of a Lewis acid, e.g. trimethylaluminium, to NH₂—XC¹ in a suitable solvent e.g. DCM, under an inert atmosphere, e.g., nitrogen, suitably at room temperature followed by addition of a compounds of formula (VI) in a compatible solvent e.g., DCM.

There is provided a further process (C) for preparing compounds of formula (I) from compounds of formula (XII) 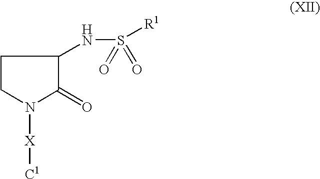
by metal-catalysed coupling of a compound of formula (XII) with a compound of formula (X) where C¹ and C² are suitable coupling groups, e.g., boronate [B(OH)₂], halide preferably iodide (I), trifluoromethanesulfonate (OTf) or stannane such as trialkyltin, and P¹ is as defined above. Suitable metal catalysts include copper (I) iodide palladium(0)or a salt thereof in the presence of a ligand, e.g., triphenylphosphine and a base, e.g., sodium carbonate or potassium carbonate, and optionally with a suitable co-solvent, e.g., water, suitably at temperature range from room temperature to 150° C. For example, when C¹ is B(OH)₂ and C² is a bromide, coupling of compounds of formula (XII) with compounds of formula (X) can be effected with tetrakis(triphenylphosphine)palladium(0) in the presence of sodium carbonate in aqueous tetrahydrofuran at 75° C.

Alternatively, where Y—C² represents a group NHR^{a}R^{b}, i.e., when C² is hydrogen bonded to a nitrogen heteroatom of Y, compounds of formula (I) may be prepared from a compound of formula (XI) by metal-catalysed coupling of a compound of formula (XII) with a compound of formula (X) where C¹ is a suitable coupling group, e.g., boronate [B(OH)₂], halide preferably iodide (I), and P¹ is as defined above. Suitable metal catalysts include palladium(0) or a salt thereof in the presence of a ligand, e.g., tri-o-tolylphosphine or a copper salt e.g., copper (I) iodide and a base, e.g., sodium tert-butoxide or potassium carbonate, and optionally with a suitable co-solvent, e.g., triethylamine, suitably at temperature range from room temperature to 150° C. For example, when C¹ is iodide, coupling of compounds of formula (XII) with compounds of formula (X) can be effected with copper (I) iodide in the presence of potassium carbonate in dimethylsulfoxide at 123° C. or tris(dibenzylideneacetone)dipalladium (0) and tri-o-tolylphosphine in the presence of sodium tert-butoxide in dioxan at 100° C.

Compounds of formula (XII) may be prepared by reacting compounds of formula (XII) with a compound of formula (III)

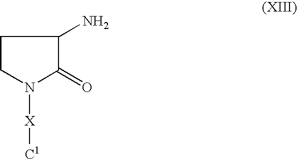

The reaction is conveniently carried out in the presence of a base, e.g. pyridine, and in a suitable solvent, e.g. DCM, suitably at room temperature.

Compounds of formula (XIII) may be prepared by deprotection of compounds of formula (IX) as described above.

There is provided a further process (D) for preparing compounds of formula (I) by coupling of compounds of formula (XIV) with compounds of formula (XV) where C³ is a suitable coupling group e.g., B(OH)₂ or halide such as bromide.

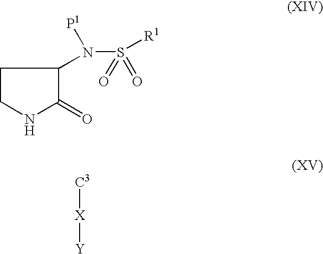

The reaction may be suitably effected under metal catalysis (e.g., copper salt such as Cu(OAc)₂ or CuCl) in the presence of a base, e.g., triethylamine or K₂CO₃, in a suitable solvent, e.g., DCM or xylene, and optionally in the presence of molecular sieves or another base e.g., tris[2-(2-methoxyethoxy)ethyl]amine at temperature range from room temperature to 200° C.

There is provided a further process (E) for the synthesis of compounds of formula (XII) by coupling of a compound of formula (XIV) with C³—X—C¹ using process described above.

Compounds of formula (XIV) may be prepared from the known 3-aminopyrrolidin-2-one or a salt thereof using methods well known to those skilled in the art, see for example Synthesis of (+−)-azetidine-2-carboxylic acid and 2-pyrrolidinone derivatives Yamada, Yasuhiro; Emori, Tomio; Kinoshita, Shinichi; Okada, Hirosuke. Fac. Eng., Osaka Univ., Suita, Japan. Agr. Biol. Chem. (1973), 37(3), 649-52.

There is provided a further process (F) for preparing compounds of formula (I) where R² is a substituent other than hydrogen, which comprises reacting a compound of formula (XVI) with a compound of formula (XVII): 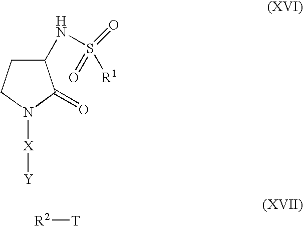
wherein R¹ and R² are defined as above and T is a suitable leaving group such as a halide, e.g. bromide . The reaction is effected in a suitable organic solvent, e.g. THF, DMF, in the presence of a base, e.g. LiHMDS (lithium hexamethyldisilylamide), potassium carbonate or sodium carbonateat a temperature range from −78° C. to +50° C., preferably −78° C. to room temperature. Furthermore, it will appreciated that the substituent R², other than hydrogen, may be introduced at various intermediate stages by methods well known to those skilled in the art.

Compounds of formula (XVI) may be prepared according to the methods described above for a compound of formula (I) where R² represents hydrogen.

It will be appreciated by those skilled in the art that compounds of formula (I) or a solvate thereof may be synthesized from appropriate intermediates via solid phase chemistry processes.

It will be appreciated by persons skilled in the art that compounds containing suitable R^{a} and R^{b} groups may be converted into their corresponding compounds where Y is a heterocycle. Examples of these interconversions can be found in Smith, M. B. and March, J., Advanced Organic Chemistry, 5^{th} Edition 2001, John Wiley & Sons.

Those skilled in the art will appreciate that in the preparation of the compound of formula (I) or a solvate thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl). Examples of suitable oxygen protecting groups may include for example alky silyl groups, such as trimethylsilyl or tert-butyldimethylsilyl; alkyl ethers such as tetrahydropyranyl or tert-butyl; or esters such as acetate.

Various intermediate compounds used in the above-mentioned process, including but not limited to certain compounds of formulae (II), (IV), (V), (IX), (XI), (XII), (XIII), and (XIV) are novel and accordingly constitute a further aspect of the present invention.

The present invention will now be further illustrated by the accompanying examples which should not be construed as limiting the scope of the invention in any way.

All publications, including but not limited to patents and patent applications, cited in this specification are herein incorporated by reference as if each individual publication were specifically and individually indicated to be incorporated by reference herein as though fully set forth.

### EXAMPLES

Abbreviations

THF Tetrahydrofuran
TFA Trifluoroacetic acid
DCM Dichloromethane
BOC t-Butyloxycarbonyl
Cbz or Z Benzyloxycarbonyl
HOBT 1-Hydroxybenzotriazole
br broad
m multiplet
q quartet
s singlet
t triplet

### Example 1

### 6-Chloro-N-{(3S)-1-[3-fluoro-2′-(methylsulfonyl)-1,1′-biphenyl-4-yl]-2-oxopyrrolidin-3-yl}naphthalene-2-sulfonamide

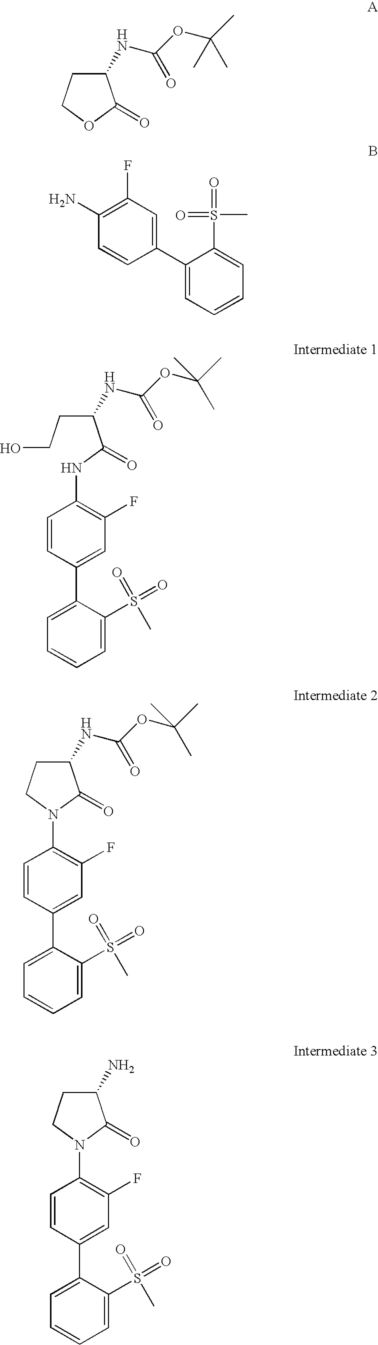

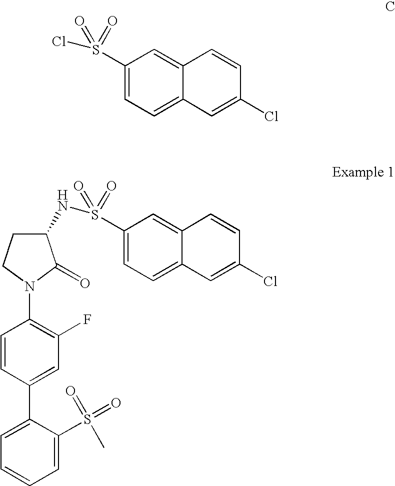

The amine, (3S)-3-Amino-1-[3-fluoro-2′-(methylsulfonyl)-1,1′-biphenyl-4-yl]pyrrolidin-2-one, (0.042 g) was dissolved in anhydrous DCM (2 ml) at room temperature. To this solution was added pyridine (0.012 ml) and (C) 6-chloro-2-naphthyl sulfonyl chloride¹. The reaction mixture was stirred at room temperature for 19 h. After this time the organic phase was washed with saturated aqueous sodium bicarbonate solution. The separated organic layer was washed and concentrated under reduced pressure to give the crude product as a yellow glass which was subsequently purified using mass directed preparative h.p.l.c. to give the title compound (0.046 g) as a white solid.

Mass spectrum: Found: MH³⁰ 573

H.p.l.c. (1) Rt 3.52 min

### Example 2

### 6-Chloro-N-{(3S)-1-[4-(dimethylamino)phenyl]-2-oxopyrrolidin-3-yl}naphthalene-2-sulfonamide

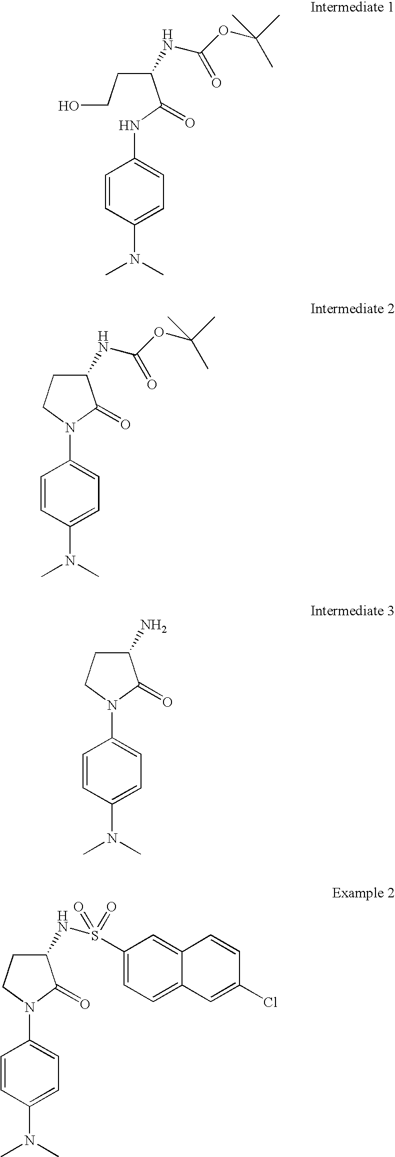

(3S)-3-Amino-1-[4-(dimethylamino)phenyl]pyrrolidin-2-one (0.0074 g) was dissolved in anhydrous DCM (2ml) at room temperature. To this solution was added pyridine (0.005 ml) and 6-chloro-2-naphthyl sulfonylchloride¹ (0.014 g). The reaction mixture was stirred at room temperature for 18 h and then evaporated under a stream of nitrogen. The resultant residue was dissolved in 1:1 mixture of DMSO:methanol (0.5 ml) and purified by mass directed preparative h.p.l.c. to give the title compound (0.007 g) as a white solid.

Mass spectrum: Found: MH⁺ 444

H.p.l.c. Rt 3.44 min

Using similar chemistry, the following compounds were prepared:

### Example 3

### (E)-2-(5-Chlorothien-2-yl)-N-((3S)-1-{5-[2-(methylsulfonyl)phenyl]pyridin-2-yl}-2-oxopyrrolidin-3-yl)ethenesulfonamide

Mass spectrum: Found: MH⁺ 538

H.p.l.c. (1) Rt 3.23 min

### Example 4

### (E)-2-(5-Chlorothien-2-yl)-N-{(3S)-1-[3-fluoro-2′-(methylsulfonyl)-1,1′-biphenyl-4-yl]-2-oxopyrrolidin-3-yl}ethenesulfonamide

Mass spectrum: Found: MH⁻ 554

H.p.l.c. (1) Rt 3.29 min

### Example 5

### 5-Chloro-N-{(3S)-1-[3-fluoro-2′-(methylsulfonyl)-1,1′-biphenyl-4-yl]-2-oxopyrrolidin-3-yl}-1-benzofuran-2-sulfonamide

Mass spectrum: Found: MH⁺ 563

H.p.l.c. (1) Rt 3.19 min

### Example 6

### N-{(3S)-1-[3-Fluoro-2′-(methylsulfonyl)-1,1′-biphenyl-4-yl]-2-oxopyrrolidin-3-yl}isoquinoline-5-sulfonamide

Mass spectrum: Found: MH⁺ 540

H.p.l.c. (1) Rt 2.78 min

### Example 7

### (E)-2-(4-Chlorophenyl)-N-{(3S)-1-[3-fluoro-2′-(methylsulfonyl)-1,1′-biphenyl-4-yl]-2-oxopyrrolidin-3-yl}ethenesulfonamide

Mass spectrum: Found: MH⁺ 549

H.p.l.c. (1) Rt 3.27 min

### Example 8

### 5′-Chloro-N-{(3S)-1-[3-fluoro-2′-(methylsulfonyl)-1,1′-biphenyl-4-yl]-2-oxopyrrolidin-3-yl}-2,2′-bithiophene-5-sulfonamide

Mass spectrum: Found: MH⁺ 611

H.p.l.c. (1) Rt 3.48 min

### Example 9

### 6-(Dimethylamino)-N-{(3S)-1-[3-fluoro-2′-(methylsulfonyl)-1,1′-biphenyl-4-yl]-2-oxopyrrolidin-3-yl}naphthalene-2-sulfonamide

Mass spectrum: Found: MH⁺ 582

H.p.l.c. (1) Rt 3.25 min

### Example 10

### N-{(3S)-1-[3-Fluoro-2′-(methylsulfonyl)-1,1′-biphenyl-4-yl]-2-oxopyrrolidin-3-yl}quinoline-8-sulfonamide

Mass spectrum: Found: MH⁺ 540

H.p.l.c. (1) Rt 2.99 min

### Example 11

### 6-Chloro-N-{(3S)-1-[3-fluoro-2′-(methylsulfonyl)-1,1′-biphenyl-4-yl]-2-oxopyrrolidin-3-yl}-1-benzothiophene-2-sulfonamide

Mass spectrum: Found: MH⁺ 579

H.p.l.c. (1) Rt 3.40 min

### Example 12

### 5-Chloro-N-{(3S)-1-[3-fluoro-2′-(methylsulfonyl)-1,1′-biphenyl-4-yl]-2-oxopyrrolidin-3yl}-1-benzothiophene-2-sulfonamide

Mass spectrum: Found: MH⁺ 579

H.p.l.c. (1) Rt 3.39 min

### Example 13

### 6-Chloro-N-[(3S)-1-(4-{2-[(dimethylamino)methyl]-1H-imidazol-1-yl}-2-fluorophenyl)-2-oxopyrrolidin-3-yl]-1-benzothiophene-2-sulfonamide formate Salt (1:1)

Mass spectrum: Found: MH⁺ 548

H.p.l.c. (1) Rt 2.56 min

### Example 14

### (1E)-2-(5-Chlorothien-2-yl)-N-[(3S)-1-(4-{2-[(dimethylamino)methyl]-1H-imidazol-1-yl}-2-fluorophenyl)-2-oxopyrrolidin-3-yl]prop-1-ene-1-sulfonamide formate salt (1:1)

Mass spectrum: Found: MH⁺ 538

### H.p.l.c. (1) Rt 2.46 min

### Example 15

### N-{(3S)-1-[2′-(Aninosulfonyl)-3-fluoro-1,1′-biphenyl-4-yl]-2-oxopyrrolidin-3-yl}-6-chloro-1-benzothiophene-2-sulfonamide

Mass spectrum: Found: MH⁺ 580

H.p.l.c. (1) Rt 3.37 min

### Example 16

### 4′-[(3S)-3-({[(1E)-2-(5-Chlorothien-2-yl)prop-1-enyl]sulfonyl}amino)-2-oxopyrrolidin-1-yl]-3′-fluoro-1,1′-biphenyl-2-sulfonamide

Mass spectrum: Found: MH⁺ 570

H.p.l.c. (1) Rt 3.33 min

### Example 17

### (E)-2-(5-Chlorothien-2-yl)-N-{(3S)-1-[5-(2-nitrophenyl)pyridin-2-yl]-2-oxopyrrolidin-3-yl}ethenesulfonamide

Mass spectrum: Found: MH⁻ 503

H.p.l.c. (1) Rt 3.50 min

### Example 18

### (E)-2-(5-Chlorothien-2-yl)-N-[(3S)-1-(3-fluoro-2′-nitro-1,1′-biphenyl-4-yl)-2-oxopyrrolidin-3-yl]ethenesulfonamide

Mass spectrum: Found: MH⁻ 520

H.p.l.c. (1) Rt 3.44 min

### Example 19

### 4′-[(3S)-3-({[(E)-2-(5-Chlorothien-2-yl)ethenyl]sulfonyl}amino)-2-oxopyrrolidin-1-yl]-3′-fluoro-N-methyl-1,1′-biphenyl-2-sulfonamide

Mass spectrum: Found: MH⁻ 568

H.p.l.c. (1) Rt 3.31 min

### Example 20

### 4′-[(3S)-3-({[(E)-2-(5-Chlorothien-2-yl)ethenyl]sulfonyl}amino)-2-oxopyrrolidin-1-yl]-3′-fluoro-1,1′-biphenyl-2-sulfonamide

Mass spectrum: Found: MH⁺ 568

H.p.l.c. (1) Rt 3.31 min

### Example 21

### (E)-2-(5-Chlorothien-2-yl)-N-[(3S)-1-(5-{2-[(methylsulfonyl)amino]phenyl}pyridin-2-yl)-2-oxopyrrolidin-3-yl]ethenesulfonamide

Mass spectrum: Found: MH⁻ 551

H.p.l.c. (1) Rt 3.23 min

### Example 22

### (E)-N-{(3S)-1-[5-(2-tert-Butylphenyl)pyridin-2-yl]-2-oxopyrrolidin-3-yl}-1-2-(5-chlorothien-2-yl)ethenesulfonamide

Mass spectrum: Found: MH⁻ 514

H.p.l.c. (1) Rt 3.90 min

### Example 23

### 5-Chloro-N-((3S)-1-{5-[2-(methylsulfonyl)phenyl]pyridin-2-yl}-2-oxopyrrolidin-3-yl)-1-benzofuran-2-sulfonamide

Mass spectrum: Found: MH⁺ 545

H.p.l.c. (1) Rt 3.33 min

### Example 24

### (E)-2-(5-Chlorothien-2-yl)-N-((3S)-2-oxo-1-{5-[2-(trifluoromethyl)phenyl]pyridin-2-yl}pyrrolidin-3yl)ethenesulfonamide

Mass spectrum: Found: MH⁻ 526

H.p.l.c. (1) Rt 3.73 min

### Example 25

### 2-{6-[(3S)-3-({[(E)-2-(5-Chlorothien-2-yl)ethenyl]sulfonyl}amino)-2-oxopyrrolidin-1-yl]pyridin-3-yl}-N,N-dimethylbenzamide

Mass spectrum: Found: MH⁻ 529

H.p.l.c. (1) Rt 3.17 min

### Example 26

### (E)-2-(5-Chlorothien-2-yl)-N-{(3S)-1-[5-(2-cyanophenyl)pyridin-2yl]-2-oxopyrrolidin-3-yl}ethenesulfonamide

Mass spectrum: Found: MH⁻ 483

H.p.l.c. (1) Rt 3.47 min

### Example 27

### 2-{6-[(3S)-3-({[(E)-2-(5-Chlorothien-2-yl)ethenyl]sulfonyl}amino)-2-oxopyrrolidin-1-yl]pyridin-3-yl}benzenesulfonamide

Mass spectrum: Found: MH⁻ 537

H.p.l.c. (1) Rt 3.18 min

### Example 28

### 2-{6-[(3S)-3-({[(E)-2-(5-Chlorothien-2yl)ethenyl]sulfonyl}amino)-2-oxopyrrolidin-(1-yl]pyridin-3-yl}-N,N-dimethylbenzenesulfonamide

Mass spectrum: Found: MH⁻ 565

H.p.l.c. (1) Rt 3,42 min

### Example 29

### 2-{6-[(3S)-3-({[(E)-2-(5-Chlorothien-2-yl)ethenyl]sulfonyl}amino)-2-oxopyrrolidin-1-yl]pyridin-3-yl}-N-methylbenzenesulfonamide

Mass spectrum: Found: MH⁺ 553

H.p.l.c. (1) Rt 3.33 min

### Example 30

### (E)-2-(5-Chlorothien-2-yl)-N-[(3S)-1-(5-{2-[methyl(methylsulfonyl)amino]phenyl}pyridin-2-yl)-2-oxopyrrolidin-3yl]ethenesulfonamide

Mass spectrum: Found: MH⁺ 567

H.p.l.c. (1) Rt 3.32 min

### Example 31

### (E)-2-(5-Chlorothien-2-yl)-N-{(3S)-1-[5-(2-isopropoxyphenyl)pyridin-2-yl]-2-oxopyrrolidin-3-yl}ethenesulfonamide

Mass spectrum: Found: MH⁺ 518

H.p.l.c. (1) Rt 3.79 min

### Example 32

6-Chloro-N-[(3S)-2-oxo-1-(5-phenylpyridin-2-yl)pyrrolidin-3-yl]naphthalene-2-sulfonamide

Mass spectrum: Found: MH⁺ 562

H.p.l.c. (1) Rt 3.42 min

### Example 33

### 5-Chloro-N-((3S)-1-{5-[2-(methylsulfonyl)phenyl]pyridin-2-yl}-2-oxopyrrolidin-3-yl)thieno[2.3-b]pyridine-2-sulfonamide

Mass spectrum: Found: MH⁺ 472

H.p.l.c. (1) Rt 3.79 min

### Example 34

### 4-Cyano-N-{(3S)-1-[3-fluoro-2′-(methylsulfonyl-1,1′-biphenyl-4-yl]-2-oxopyrrolidin-3-yl}benzenesulfonamide

Mass spectrum: Found: MNH₄⁺ 531

H.p.l.c. (1) Rt 3.17 min

### Example 35

### 3-Cyano-N-{(3S)-1-[3-fluoro-2′-(methylsulfonyl)-1,1′-biphenyl-4-yl]-2-oxopyrrolidin-3-yl}benzenesulfonamide

Mass spectrum: Found: MNH₄⁺ 531

H.p.l.c. (1) Rt 3.16 min

### Example 36

### 6-Chloro-N-{(3S)-1-[3-fluoro-2′-(methylsulfonyl)-1,1′-biphenyl-4yl]-2-oxopyrrolidin-3-yl}-1-benzofuran-2-sulfonamide

Mass spectrum: Found: MH⁺ 563

H.p.l.c. (1) Rt 3.35 min

### Example 37

### 6-Chloro-N-{(3S)-1-[3-fluoro-2′-(methylsulfonyl)-1,1′-biphenyl-yl]-2-oxopyrrolidin-3-yl}thieno[3,2b]pyridine-2-sulfonamide

Mass spectrum: Found: MH⁺ 580

H.p.l.c. (1) Rt 3.24 min

### Example 38

### 5-Chloro-N-{(3S)-1-[3-fluoro-2′-(methylsulfonyl)-1,1′-biphenyl-4-yl]-2-oxopyrrolidin-3-yl}thieno[3,2-b]pyridine-2-sulfonamide

Mass spectrum: Found: MH^{+ 580}

H.p.l.c. (1) Rt 3.19 min

### Example 39

### (1E)-2-(5-Chlorothien-2-yl)-N-{(3S)-1-[3-fluoro-2′-(methylsulfonyl)-1,1′-biphenyl-4yl]-2-oxopyrrolidin-3-yl}prop-1-ene-1-sulfonamide

Mass spectrum: Found: MNH₄⁺ 586

H.p.l.c. (1) Rt 3.37 min

### Example 40

### tert-Butyl [{[(E)-2-(5-Chlorothien-2-yl)ethenyl]sulfonyl}((3S)-1-{5-[2-(methylsulfonyl)phenyl]pyridin-2-yl}-2-oxopyrrolidin-3-yl)amino]acetate

Example 3 (0.05 g) was dissolved in anhydrous DMF (1 ml) in a reactivial. tert-Butoxycarbonyl]bromoacetate (0.029 g) was added, followed by potassium carbonate (0.037 g) and the mixture was stirred at ambient temperature for 21 h. The reaction mixture was diluted with DCM, and washed with aqueous saturated sodium bicarbonate. The organic layer was separated and concentrated under reduced pressure to give the title compound (0.049 g) as a clear oil.

Mass spectrum: Found: MH⁺ 652

H.p.l.c. (1) Rt 3.81 min

Using similar chemistry, the following compounds were prepared:

### Example 41

### [{[(E)-2-(5-Chlorothien-2-yl)ethenyl]sulfonyl}((3S)-1-{5-[2-(methylsulfonyl) phenyl]pyridin-2-yl}-2-oxopyrrolidin-3-yl)amino]acetic acid

Mass spectrum: Found: MH⁺ 610

H.p.l.c. (1) Rt 3.41 min

### Example 42

### (E)-2-(5-Chlorothien-2-yl)-N-((3S)-1-{5-[2-(methylsulfonyl)Phenyl]pyridin-2-yl}-2-oxopyrrolidin-3-yl)-N-(2-morpholin-4-ylethyl)ethenesulfonamide Formate Salt (1:1)

Mass spectrum: Found: MH⁺ 651

H.p.l.c. (1) Rt 2.64 min

### Example 43

### 2-[{[(E)-2-(5-Chlorothien-2-yl)ethenyl]sulfonyl}((3S)-1-{5-[2-(methylsulfonyl)phenyl]pyridin-2-yl}-2-oxopyrrolidin-3-yl)amino]acetamide

Mass spectrum: Found: MH⁺ 595

H.p.l.c. (1) Rt 3.11 min

### Example 44

### tert-Butyl [(E)-2-(5-chlorothien-2-yl)ethenyl]sulfonly{(3S)-1-[3-fluoro-2′-(methylsulfonyl)-1,1′-biphenyl-4yl]-2-oxopyrrolidin-3-yl}carbamate

Mass spectrum: Found: MH⁺ 655

H.p.l.c. (1) Rt 3.69 min

### Example 45

### (E)-2-(5-Chlorothien-2-yl)-N-{(3S)-1-[3-fluoro-2′-(methylsulfonyl)-1,1′-biphenyl-4-yl]-2-oxopyrrolidin-3-yl}-N-(2-morpholin-4-ylethyl)ethenesulfonamide

Mass spectrum: Found: MH⁺ 668

H.p.l.c. (1) Rt 2.88 min

### Example 46

### 2-({[(E)-2-(5-Chlorothien-2-yl)ethenyl]sulfonyl}{(3S)-1-[3-fluoro-2′-(methylsulfonyl-1,1′-biphenyl-4-yl]-2-oxopyrrolidin-3-yl}amino)acetamide

Mass spectrum: Found: MH⁺ 612

H.p.l.c. (1) Rt 3.33 min

### Example 47

### tert-Butyl ({[(E)-2-(5-chlorothien-2-yl)ethenyl]sulfonyl}{(3S)-1-[3-fluoro-2′-(methylsulfonyl)-1,1′-biphenyl-4-yl]-2-oxopyrrolidin-3-yl}amino)acetate

Mass spectrum: Found: MH⁺ 669

H.p.l.c. (1) Rt 3.91 min

### Example 48

### {[(E)-2-(5-Chlorothien-2-yl)(ethenyl]sulfonyl}((3S)-1-{5-[2-(methylsulfonyl)phenyl]pyridin-2-yl}-2-oxopyrrolidin-3-yl)amino]acetic Acid

Example 40 (0.0497 g) was dissolved in anhydrous DCM (0.5 ml). Trifluoroacetic acid (0.50 ml) was added and the mixture was stirred at ambient temperature for 1.5 h. The reaction mixture was concentrated under reduced pressure. The residue was then purified using SPE (silica, eluting with DCM, diethyl ether, ethyl acetate and acetonitrile) to give the title compound (0.03 g) as a cream solid.

Mass spectrum: Found: MH⁺ 596

H.p.l.c. (1) Rt 3.53 min

Using similar chemistry and Example 47, the following was prepared:

### Example 49

### ({[(E)-2-(5-Chlorothien-2-yl)ethenyl]sulfonyl}{(3S)-1[-3-fluoro-2′-(methylsulfonyl-1,1′-biphenyl-4-yl]-2-oxopyrrolidin-3-yl}amino)acetic Acid

Mass spectrum: Found: MH⁺ 613

H.p.l.c. (1) Rt 3.53 min

### Example 50

### 4′-[(S)-3-(6-Chloro-naphthalene-2-sulphonylamino)-2-oxo-pyrrolidin-1-yl]-3′-fluoro-biphenyl-3-carboxylic Acid Amide

A solution of 6-chloro-naphthalene-2-sulphonic acid [(S)-1-(2-fluoro-4-iodophenyl)-2-oxo-pyrrolidin-3-yl]-amide (0.083 g), 3-(aminocarbonyl)phenyl boronic acid (0.03 g) and tetrakistriphenylphospinepalladium(0) (0.01 g) in DME (5 ml) containing 0.5M sodium carbonate solution (1 ml) was degassed with nitrogen and then stirred for 18 h at ambient temperature. The mixture was then heated at 80° C. for 4 h, allowed to cool and concentrated under reduced pressure. The residue was purified using flash column chromatography (silica, eluting with DCM followed by ethyl acetate) to give the title compound (0.066 g) as a cream solid.

Mass spectrum: Found: MH⁺ 538

H.p.l.c. (1) Rt 3.31 min

Using similar chemistry, the following was prepared:

### Example 51

### 6-Chloro-naphthalene-2-sulphonic Acid [(S)-1-[5-(2-methylsulphanylphenyl)-thiazol-2-yl]-2-oxo:pyrrolidin-3-yl]amide

Mass spectrum: Found: MH⁺ 530

H.p.l.c. (1) Rt 3.58 min

### Example 52

### 6-Chloro-naphthalene-2-sulphonic Acid [(S)-1-[5-(2-methanesulphonylphenyl)-thiazol-2-yl]-2-oxo-pyrrolidin-3-yl]amide

To a solution of Example 51 (0.085 g) in DCM (5 ml) was added meta-chloroperbenzoic acid (0.102 g) and the solution was stirred for 4 h then washed with saturated sodium carbonate solution. The organic layer was separated, dried (over magnesium sulphate) and concentrated under reduced pressure. The residue was purified using flash column chromatography (silica, eluting with DCM, DCM:ethyl acetate 5:1) to give the title compound (0.032 g) as a white solid.

Mass spectrum: Found: MH⁺ 562

H.p.l.c. (1) Rt 3.35 min

### Example 53

### 3-(Aminomethyl)-N-{(3S)-1-[3-fluoro-2′-(methylsulfonyl)-1,1′-biphenyl-4-yl]-2-oxopyrrolidin-3-yl}benzenesulfonamide

Example 35 (0.109 g) was dissolved in ethanol (4.5 ml) and dilute hydrochloric acid (2N, 0.5 ml). To this solution, was added 20% palladium hydroxide on carbon (0.0086 g) and the resulting suspension was stirred under hydrogen (60 psi) at 60° C. for 60 h. The catalyst was filtered through Celite® and the volatile components of the filtrate removed under reduced pressure. The residue was purified by ion exchange solid phase extraction (eluting with with methanol and then 10% aqueous ammonia in methanol) to give the title compound (0.066 g) as an off-white gum.

Mass spectrum: Found: MH⁺ 518

H.p.l.c. (1) Rt 2.17 min

Using Example 34 and similar chemistry, the following was prepared:

### Example 54

### 4-(Aminomethyl)-N-{(3S)-1-[3-fluoro-2′-(methylsulfonyl)-1,1′-biphenyl-4-yl]-2-oxopyrrolidin-3-yl}benzenesulfonamide

Mass spectrum: Found: MH⁺ 518

H.p.l.c. (1) Rt 2.24 min

### Example 55

### 6-Chloro-N-[(3S)-1-(2-fluoro-4-pyridin-4-ylphenyl)-2-oxopyrrolidin-3-yl]naphthalene-2-sulfonamide

A solution of Intermediate 70 (0.242 g) and tetrakistriphenylphospinepalladium(0) (0.025 g) in dimethoxyethane (purged with nitrogen, 10 ml) was stirred at room temperature for 10 min under nitrogen. Pyridine-4-boronic acid (0.66 g) was added followed by 0.5M sodium carbonate (2.7 ml). The resulting mixture was heated for 18 h at 80-85° C. The cooled solution was diluted with DCM and filtered through a hydrophobic frit. The filtrate was added to a SCX-2 SPE column (silica, eluting with methanol and then 2M ammonia in methanol) to give the title compound (0.14 g) as a peach coloured solid.

Mass spectrum: Found: MH⁺ 496

H.p.l.c. (1) Rt 3.08 min

Using similar chemistry, the following were prepared:

### Example 56

### 6-Chloro-N-{(3S)-1-[4-(2,4-dimethoxypyrimidin-5-yl)-2-fluorophenyl]oxopyrrolidin-3-yl}naphthalene-2-sulfonamide

Mass spectrum: Found: MH⁺ 557

H.p.l.c. (1) Rt 3.46 min

### Example 57

### 6-Chloro-N-[(3S)-1-(2-fluoro-4-pyridin-3ylphenyl)-2-oxopyrrolidin-3-yl]naphthalene-2-sulfonamide

Mass spectrum: Found: MH⁺ 496

H.p.l.c. (1) Rt 3.22 min

### Example 58

### 6-Chloro-N-{(3S)-1-[2-fluoro-4-(6-methoxypyridin-3-yl)phenyl]-2-oxopyrrolidin -3-yl}naphthalene-2-sulfonamide

Mass spectrum: Found: MH⁺ 526

H.p.l.c. (1) Rt 3.53 min

### Example 59

### 6-Chloro-N-{(3S)-1-[2-fluoro-4-(4-propylpyridin-3-yl)phenyl]-2-oxopyrrolidin-3-yl}naphthalene-2-sulfonamide

Mass spectrum: Found: MH³⁰ 538

H.p.l.c. (1) Rt 3.48 min

### Example 60

### 6-Chloro-N-((3S)-1-{2-fluoro-4-[6-(methylthio)pyridin-3-yl]phenyl}-2-oxopyrrolidin-3-yl)naphthalene-2-sulfonamide

Mass spectrum: Found: MH⁺ 542

H.p.l.c. (1) Rt 3.74 min

### Example 61

### N-{(3S)-1-[4-(5-Bromopyridin-3-yl)-2-fluorophenyl]-2-oxopyrrolidin-3-yl}-6-chloronaphthalene-2-sulfonamide

Mass spectrum: Found: MH⁺ 576

H.p.l.c. (1) Rt 3.68 min

### Example 62

### 6-Chloro-N-{(3S)-1-[2-fluoro-4-(4-methoxypyridin-3-yl)phenyl]-2-oxopyrrolidin-3-yl}naphthalene-2-sulfonamide

Mass spectrum: Found: MH⁺ 526

H.p.l.c. (1) Rt 2.91 min

### Example 63

### 6-Chloro-N-[(3S)-1-(2-fluoro-4-pyrimidin-5-ylphenyl)-2-oxopyrrolidin-3-yl]naphthalene-2-sulfonamide

Mass spectrum: Found: MH⁺ 497

H.p.l.c. (1) Rt 3.12 min

### Example 64

### N-{(3S)-1-[3′-(Aminomethyl)-3-fluoro-1,1′-biphenyl-4-yl]-2-oxopyrrolidin-3-yl})-6-chloronaphthalene-2-sulfonamide

Mass spectrum: Found: MH⁺ 524

H.p.l.c. (1) Rt 2.79 min

### Example 65

### 6-Chloro-N-{(3S)-1-[2-fluoro-4-(3-furyl)phenyl]-2-oxopyrrolidin-3-}naphthalene-2-sulfonamide

Mass spectrum: Found: MH⁺ 485

H.p.l.c. (1) Rt 3.55 min

### Example 66

### 6-Chloro-N-{(3S)-1-[2-fluoro-4-(4-methylthien-2-yl)1phenyl]-2-oxopyrrolidin-3-yl}naphthalene-2-sulfonamide

Mass spectrum: Found: MH⁺ 515

H.p.l.c. (1) Rt 3.79 min

### Example 67

### 6-Chloro-N-[(3S)-1-(2-fluoro-4-thien-3-ylphenyl)-2-oxopyrrolidin-3-yl]naphthalene-2-sulfonamide

Mass spectrum: Found: MH⁺ 501

H.p.l.c. (1) Rt 3.90 min

### Example 68

### 6-Chloro-N-{(3S)-1-[2-fluoro-4-(5-methylthien-2-yl)phenyl]-2-oxopyrrolidin-3-yl}naphthalene-2-sulfonamide

Mass spectrum: Found: MH⁺ 515

H.p.l.c. (1) Rt 3.70 min

### Example 69

### 6-Chloro-N-{(3S)-1-[2-fluoro-4-(4-methylthien-3-yl)phenyl]-2-oxopyrrolidin-3-yl}naphthalene-2-sulfonamide

Mass spectrum: Found: MH⁺ 515

H.p.l.c. (1) Rt 3.86 min

### Example 70

### 6-Chloro-N-{(3S)-1-[2-fluoro-4-(3-formylthien-2-yl)phenyl]-2-oxopyrrolidin-3-yl}naphthalene-2-sulfonamide

Mass spectrum: Found: MH⁺ 529

H.p.l.c. (1) Rt 3.62 min

### Example 71

### 6-Chloro-N-{(3S)-1-[4-(5-chlorothien-2-yl)-2-fluorophenyl]-2-oxopyrrolidin-3-yl}naphthalene-2-sulfonamide

Mass spectrum: Found: MH⁺ 535

H.p.l.c. (1) Rt 4.01 min

### Example 72

### 6-Chloro-N-{(3S)-1-[4-(3,5-dimethylisoxazol-4-yl)-2-fluorophenyl]-2-oxopyrrolidin-3-yl}naphthalene-2-sulfonamide

Mass spectrum: Found: MH⁺ 514

H.p.l.c. (1) Rt 3.40 min

### Example 73

### 6-Chloro-N-{(3S)-1-[2-fluoro-4-(5-methyl-2-furyl)phenyl]-2-oxopyrrolidin-3-yl}naphthalene-2-sulfonamide

Mass spectrum: Found: MH⁺ 499

H.p.l.c. (1) Rt 3.70 min

### Example 74

### 6-Chloro-N-[(3S)-1-(3-fluoro-1,1′-biphenyl-4-yl)-2-oxopyrrolidin-3-yl]naphthalene-2-sulfonamide

Mass spectrum: Found: MH⁺ 495

H.p.l.c. (1) Rt 3.68 min

### Example 75

### (E)-2-(5-Chlorothien-2-yl)-N-[(3S)-1-(4-{2-[(dimethylamino)methyl]-1H-imidazol-1yl}-2-fluorophenyl)-2-oxopyrrolidin-3-yl]ethenesulfonamide Bis(trifluoroacetate)

To a solution of Intermediate 72 (0.245 g) in DCM (10 ml) was added trifluoroacetic acid (1 ml). The solution was stirred for 1 h, and then concentrated under reduced pressure. The residue was dissolved in acetonitrile (10 ml). A 5 ml aliquot was taken and diluted with acetonitrile (5 ml). To this was added N,N-diisopropylethylamine (0.332 ml) and (E)-2-(5-chlorothien-2-yl)ethenesulfonyl chloride (0.071 g). After stirring for 18 h at room temperature under nitrogen, the reaction mixture was loaded onto a SCX-2 SPE column (silica, eluting with methanol and then 0.5M ammonia in methanol) to give an impure sample of the title compound. Further purification using mass directed preparative h.p.l.c. provided a pure sample of the title compound (0.052 g) as a white solid.

Mass spectrum: Found: MH⁺ 524

H.p.l.c. (1) Rt 2.45 min

¹H NMR in DMSO: δ10.08 (1H, br.s), 8.08(1H, d), 7.70-7.62(2H, m), 7.61(1H, d), 7.51(1H, d), 7.43(1H, d), 7.42(1H, dd), 7.25(1H, d), 7.20(1H, d), 7.00(1H, d), 4.48(2H, s), 4.29(1H, m), 3.79(1H, m), 3.68(1H, t), 2.81(6H, s), 2.54(1H, m), 2.05(1H, m) ppm.

### Example 76

### 6-Chloro-N-{(3S)-1-[2-fluoro-4-(1-oxidopyridin-4-yl)phenyl]-2-oxopyrrolidin-3-yl}naphthalene-2-sulfonamide

To a solution of Example 55 (0.045 g) in DCM was added (57-86%) 3-chloroperbenzoic acid (0.031 g). The mixture was stirred for 18 h at room temperature then diluted with DCM and washed with 10% sodium bicarbonate. The organic phase was passed through a hydrophobic frit and loaded onto a SPE column (silica, eluting with diethyl ether, ethyl acetate, acetone and finally methanol) to give the title compound (0.025 g) as a tan coloured solid.

Mass spectrum: Found: MH⁺ 512

H.p.l.c. (1) Rt 3.06 min

### Example 77

### 6-Chloro-N-{(3S)-1-[2-fluoro-4-(1-methyl-1H-imidazol-2-yl)phenyl]-2-oxopyrrolidin-3-yl}naphthalene-2-sulfonamide

A mixture of 2-bromo-1-methylimidazole (0.161g), potassium acetate (0.294 g), 1,1′-bis(diphenylphosphino)ferrocene dichloro palladium(II) complex with DCM (0.041 g) and bispinacolatodiboron (0.279 g) in dimethoxyethane (12.5 ml, degassed) was heated at 80° C. for 6 h. The reaction mixture was concentrated under reduced pressure and the residue partitioned between ethyl acetate and 50% saturated sodium chloride solution. The separated organic phase was dried (over magnesium sulphate) and concentrated under reduced pressure to give 1-methyl-2-(4,4,5,5-tetramethyl-1,3,2-dioxaborolan-2-yl)-1H-imidazole (0.358 g). This was dissolved in dimethoxyethane (17 ml, degassed) and tetrakistriphenylphospinepalladium(0) (0.115 g), was added. After 5 min, potassium acetate (0.294 g), Intermediate 70 (0.46 g) and water (4 ml) were added and the mixture heated at 80° C. for 84 h. The reaction mixture was concentrated under reduced pressure and the residue partitioned between DCM and water. The organic phase was passed through a hydrophobic frit and loaded onto a SPE SCX-2 column (silica, eluting with methanol and then 0.5M ammonia in methanol) to give the title compound (0.045 g) as a brown solid.

Mass spectrum: Found: MH⁺ 499

H.p.l.c. Rt 2.65 min

### Example 78

### 6-Chloro-N-{(3S)-1-[4-(2-chloropyridin-3-yl)-2-fluorophenyl]-2-oxopyrrolidin-3-yl}naphthalene-2-sulfonamide

To a solution of Intermediate 74 (0.067 g) in DCM (20 ml) was added trifluoroacetic acid (1 ml). After stirring for 1.5 h the solution was concentrated under reduced pressure to give (3S)-3-amino-1-[4-(2-chloropyridin-3-yl)-2-fluorophenyl-]pyrrolidin-2-one trifluoroacetate (0.083 g). This material was suspended in acetonitrile (7.5 ml), and N,N-diisopropylethylamine (0.116 ml) and 6-chloro-2-napthyl sulfonyl chloride¹ (0.044 g) were added and the resultant solution was stirred at room temperature for 72 h under nitrogen. After removal of the solvent, the residue was partitioned between DCM and saturated sodium hydrogen carbonate solution. The organic phase was dried (using a hydrophobic frit) and loaded onto a SPE column (silica, eluting with DCM, diethyl ether, ethyl acetate) to give the title compound (0.023 g) as a white solid

Mass spectrum: Found: MH⁺ 530

H.p.l.c. Rt 3.44 min

### Example 79

### 6-Chloro-N-{(3S)-1-[4-(2-cyanopyridin-3-yl)-2-fluorophenyl-]-2-oxopyrrolidin-3-yl}naphthalene-2-sulfonamide

Using Intermediate 75 and the synthetic procedure described for Example 78 above, provided the title compound (0.029 g) as a pale gum.

Mass spectrum: Found: MH⁺ 521

H.p.l.c. Rt 3.36 min

### Example 80

### (E)-N-{(3S)-1-[4-(3-Chloropyridin-4-yl)-2-fluorophenyl]-2-oxopyrrolidin-3-yl}-2-(5-chlorothien-2-yl)ethenesulfonamide

A suspension of Intermediate 76 (0.205 g) in acetonitrile (10 ml) was treated with N,N-diisopropylethylamine (0.24 ml) and the resulting solution cooled in an ice bath. (E)-2-(5-chlorothien-2-yl)ethenesulfonyl chloride (0.068 g) was added and the solution left to warm to room temperature over 18 h The reaction mixture was concentrated under reduced pressure and the residue purified using SPE (silica, eluting with DCM, diethyl ether, and finally ethyl acetate) to give an impure sample of the title compound. Further purification using SPE (eluting with methanol and then 0.5M ammonia in methanol) gave the title compound (0.100 g) as a tan oil.

Mass spectrum: Found: MH⁺ 512

H.p.l.c. Rt 3.36 min

### Example 81

### 6-Chloro-N-[(3S)-1-(2-fluoro-4-pyrimidin-2-ylphenyl)-2-oxopyrrolidin-3-yl]naphthalene-2-sulfonamide

Using Intermediate 77, 6-chloro-2-napthyl sulfonyl chloride, and the synthetic procedure described for Example 78, provided the title compound as a buff solid,

Mass spectrum: Found: MH⁺ 497

H.p.l.c. Rt 3.45 min

### Example 82

### 6-Chloro-N-{(3S)-1-[4-(3-chloropyridin-2-yl)-2-fluorophenyl]-2-oxopyrrolidin-3-yl}naphthalene-2-sulfonamide

Using Intermediate 78, 6-chloro-2-napthyl sulfonyl chloride, and the synthetic procedure described for Example 78, provided the title compound as a white foam.

Mass spectrum: Found: MH⁺ 530

H.p.l.c. Rt 3.55 min

### Example 83

### 6-chloro-N-{(3S)-1-[4-(3-Chloropyridin-4-yl)-2-fluorophenyl]-2-oxopyrrolidin-3-yl}naphthalene-2-sulfonamide

Using Intermediate 71, 3-chloro-4-pyridineboronic acid pentahydrate, and the synthetic procedure described for Example 55, provided the title compound as an off white solid.

Mass spectrum: Found: MH⁺ 530

H.p.l.c. Rt 3.46 min

### Example 84

### 6Chloro-N-{(3S)-1-[2-fluoro-4-(1-methyl-1H-imidazol-4-yl)phenyl]-2-oxopyrrolidin-3-yl}naphthalene-2-sulfonamide Formate

Using Intermediate 70, 4-bromo-1-methyl-1H-imidazole, and the synthetic procedure described for Example 77, provided the title compound as a tan gum.

Mass spectrum: Found: MH⁺ 499

H.p.l.c. Rt 2.81 min

### Example 85

### 6-Chloro-N-{(3S)-1-[2-fluoro-4-(1-methyl-1H-imidazol-5-yl)phenyl]-2-oxopyrrolidin-3-yl}naphthalene-2-sulfonamide

Using Intermediate 70, 5-bromo-1-methyl-1H-imidazole and the synthetic procedure described for Example 77, provided the title compound as a maroon oil.

Mass spectrum: Found: MH⁺ 499

H.p.l.c. Rt 2.81 min

### Example 86

### 2-(5-Chlorothien-2-yl)-N-{(3S)-1-[3-fluoro-2′-(methylsulfonyl)-1,1′-biphenyl-4-yl]-2-oxopyrrolidin-3-yl}-1,3-thiazole-5-sulfonamide

To a solution of Intermediate 65 (0.1332 g) in dry THF (3 ml) at −78° C. under nitrogen, n-butyllithium (1.6M in hexanes, 0.46 ml) was added. After stirring for 25 min, sulphur dioxide was condensed into the reaction for about 10 mi and the mixture was then allowed to warm to room temperature and concentrated under reduced pressure. The resultant solid was stirred with N-chlorosuccinimide (0.108 g) in dry DCM (5 ml) for 5 h, filtered and concentrated under reduced pressure to give the crude 2-(5′-chlorothien-2′-yl)-2-thiazolyl-5-sulfonyl chloride (0.203 g) as a yellow solid.

A mixture of (3S)-3-amino-1-[3-fluoro-2′-(methylsulfonyl)-1,1′-biphenyl-4-yl]pyrrolidin-2-one (0.019 g), the crude 2-(5′-chlorothien-2′-yl)-2-thiazolyl-5-sulfonyl chloride (0.032 g) and pyridine (0.015 ml) in acetonitrile (0.5 ml) was sonicated for 2 min and stirred at room temperature for 18 h. The reaction mixture was evaporated under a stream of nitrogen to give a brown residue (0.040 g) that was purified using mass directed preparative h.p.l.c. to give the title compound (0.0069 g) as fine off white crystals.

Mass spectrum: Found: MH⁺ 612

H.p.l.c. (1) Rt 3.48 min

Using 2-chlorothieno[3,2-b]thiophene* and similar chemistry, the following compounds were prepared and isolated from the same reaction:

*Bugge, Andreas, Chem. Scr. (1972), 2(3), 137-42

### Example 87

### 5-Chloro-N-{(3S)-1-[3-fluoro-2′-(methylsulfonyl)-1,1′-biphenyl-4-yl]-2-oxopyrrolidin-3-yl}thieno[3.2-b]thiophene-2-sulfonamide

Mass spectrum: Found: MNH₄⁺ 602

H.p.l.c. (1) Rt 3.39 min

### Example 88

### 2-Chloro-N-{(3S)-1-[3-fluoro-2′-(methylsulfonyl)-1,1′-biphenyl-4-yl]-2-oxopyrrolidin-3-yl}thieno[3.2-b]thiophene-3-sulfonamide

Mass spectrum: Found: MNH₄⁺ 602

H.p.l.c. (1) Rt 3.28 min

### Example 89

### 6-Chloro-N-[(3S)-1-(2-fluoro-4-iodophenyl)-2-oxopyrrolidin-3-yl]naphthalene-2-sulfonamide

(3S)-3-Amino-1-(2-fluoro-4-iodophenyl)pyrrolidin-2-one hydrochloride (2.67 g) was suspended in DCM (80 ml). N,N-diisopropylethylamine (2.13 g) was added, followed by 6-chloro-napthyl-2-sulfonyl chloride¹ (1.97 g). The solution was stirred for 18 h at room temperature, then poured onto SCX-2 SPE columns and washed with DCM. The DCM was concentrated under reduced pressure Crystallisation from methanol/diethyl ether (1:1) gave the title compound (1.4 g) as white needles. Further material (1.56 g) was obtained from the mother liquors by purification by Biotage™ chromatography (eluting with DCM then cyclohexane:ethyl acetate 2:1).

Mass spectrum: Found: MH⁺ 545

H.p.l.c. (1) Rt 3.60 min

Using similar chemistry, the following were also prepared:

### Example 90

### (E)-2-(5-Chlorothien-2-yl)-N-[(3S)-1-(5-iodopyridin-2yl)oxopyrrolidin-3-yl]ethenesulfonamide

Mass spectrum: Found: MH⁺ 510

H.p.l.c. (1) Rt 3.54 min

### Example 91

### 6-Chloro-N-[(3S)-1-(2-fluoro-4-iodophenyl)-2-oxopyrrolidinyl]-1-benzothiophene-2-sulfonamide

Mass spectrum: Found: MH⁺ 548

H.p.l.c. (1) Rt 3.65 min

### Example 92

### 5′-Chloro-N-[(3S)-1-(2-fluoro-4-iodophenyl)-2-oxopyrrolidin-3-yl]-2,2′-bithiophene-5-sulfonamide

Mass spectrum: Found: MH⁻ 580

H.p.l.c. (1) Rt 3.8 min

### Example 93

### 2-(5-Chloro-2-thienyl)-N-[(3S)-1-(2-fluoro-4-iodophenyl)-2-oxopyrrolidinyl]ethanesulfonamide

Mass spectrum: Found: MH⁺ 528

H.p.l.c. (1) Rt 3.59 min

### Example 94

### 6-Chloro-N-[(3R)-1-(2-fluoro-4-nitrophenyl)-2-oxopyrrolidinyl]-1-benzothiophene-2-sulfonamide

Mass spectrum: Found: MH⁻ 468

H.p.l.c. (1) Rt 3.45 min

### Example 95

### (E)-2-(5-Chloro-2-thienyl)-N-[(3S)-1-(2-fluoro-4-nitrophenyl)-2-oxopyrrolidinyl]ethenesulfonamide

Mass spectrum: Found: MH⁻ 444

H.p.l.c. (1) Rt 3.30 min

### Example 96

### 5′-Chloro-N-[(3S)-1-(2-fluoro-4-nitrophenyl)-2-oxopyrrolidin-3-yl]-2,2′-bithiophene-5-sulfonamide

Mass spectrum: Found: MH⁻ 500

H.p.l.c. (1) Rt 3.60 min

### Example 97

### 6-Chloro-N-[(3S)-1-(4-cyano-2-fluorophenyl)-2-oxopyrrolidin-3-yl]-1-benzothiophene-2-sulfonamide

Mass spectrum: Found: MH⁻ 447

H.p.l.c. (1) Rt 3.36 min

### Example 98

### (E)-2-(5-Chlorothien-2-yl)-N-[(3S)-1-(4-cyano-2-fluorophenyl)-2-oxopyrrolidin-3-yl]ethenesulfonamide

Mass spectrum: Found: MH⁻ 424

H.p.l.c. (1) Rt 3.19 min

### Example 99

### 2-(5-Chlorothien-2-yl)-N-[(3S)-1-(4-cyano-2-fluorophenyl)-2-oxopyrrolidin-3-yl]ethanesulfonamide

Mass spectrum: Found: MH⁻ 426

H.p.l.c. (1) Rt 3.23 min

### Example 100

### (E)-2-(5-Chloro-2-thienyl)-N-[(3S)-1-(2-fluoro-4-isopropenylphenyl)-2-oxopyrrolidinyl]ethenesulfonamide

Mass spectrum: Found: MH⁺ 441

H.p.l.c. (1) Rt 3.53 min

### Example 101

### 6-Chloro-N-[(3S)-1-(2-fluorophenyl)-2-oxopyrrolidin-3-yl]naphthalene-2-sulfonamide

Obtained as a biproduct from a boronic acid coupling. Purification by preparative h.p.l.c. gave the title compound as a white solid.

LC/MS ESI Rt 1.51 mins no ion seen

### Example 102

### N-[(3S)-1-(4-Bromo-2-fluorophenyl)-2-oxopyrrolidinyl]-6-chloro-2-naphthalenesulfonamide

Mass spectrum: Found: MH⁺ 501

H.p.l.c. (1) Rt 3.84 min

### Example 103

### 6-Chloro-N-{(3S)-1-[3-fluoro-4-(4-morpholinyl)phenyl]-2-oxopyrrolidinyl}-2-naphthalenesulfonamide

Mass spectrum: Found: MH⁺ 504

H.p.l.c. (1) Rt 3.41 min

### Example 104

### 4-[(3S)-3-({[(E)-2-(5-Chlorothien-2-yl)ethenyl]sulfonyl}amino)-2-oxopyrrolidin-1-yl]-3-fluoro-N,N-dimethylbenzamide

Mass spectrum: Found: MH³¹ 470

H.p.l.c. (1) Rt 2.89 min

### Example 105

### (E)-2-(5-Chloro-2-thienyl)-N-{(3S)-1-[2-fluoro-4-(1-pyrrolidinylcarbonyl)phenyl]-2-oxopyrrolidinyl}ethenesulfonamide

Mass spectrum: Found: MH+ 498

H.p.l.c. (1) Rt 3.01 min

### Example 106

### 6-[(3S)-3-({[(E)-2-(5-Chlorothien-2-yl)ethenyl]sulfonyl}amino)-2-oxopyrrolidin-1-yl]nicotinamide

Mass spectrum: Found: MH⁺ 427

H.p.l.c. (1) Rt 2.78 min

### Example 107

### 4-[(3S)-3-({[(E)-2-(5-Chlorothien-2-yl)ethenyl]sulfonyl}amino)-2-oxopyrrolidin-1-yl]-3-fluorobenzamide

Mass spectrum: Found: MH⁻ 442

H.p.l.c. (1) Rt 3.80 min

### Example 108

### 4-[(3S)-3-({[(E)-2-(5-Chlorothien-2-yl)ethenyl]sulfonyl}amino)-2-oxopyrrolidin-1-yl]-3-fluoro-N-methylbenzamide

Mass spectrum: Found: MH⁻ 456

H.p.l.c. (1) Rt 2.86 min

### Example 109

### 4-((3S)-3-{[(6-Chloro-1-benzothien-2-yl)sulfonyl]amino}-2-oxopyrrolidin-1-yl)-3-fluoro-N,N-dimethylbenzamide

Mass spectrum: Found: MH⁻ 494

H.p.l.c. (1) Rt 3.08 min

### Example 110

### 4-[(3S)-3({[(1E)-2-(5-Chlorothien-2-yl)prop-1-enyl]sulfonyl}amino)-2-oxopyrrolidin-1-yl]-3-fluoro-N,N-dimethylbenzamide

Mass spectrum: Found: MH⁻ 484

H.p.l.c. (1) Rt 2.98 min

### Example 111

### 4-((3S)-3-{[(6-Chloro-1-benzothien-2-yl)sulfonyl]amino}-2-oxopyrrolidin-1-yl)-3-fluoro-N-isopropyl-N-methylbenzamide

Mass spectrum: Found: MH⁻ 522

H.p.l.c. (1) Rt 3.27 min

### Example 112

### (E)-N-[(3S)-1-(4-acetyl-2-fluorophenyl))-2-oxopyrrolidin-3-yl]-2-(5-chlorothien-2-yl)ethenesulfonamide

Mass spectrum: Found: MH⁻ 441

H.p.l.c. (1) 3.16 min

Using similar chemistry, the following was prepared:

### Example 113

### (E)-N-[(3S)-1-(5-Acetylpyridin-2-yl)-2-oxopyrrolidin-3yl]-2-(5-chlorothien-2-yl)ethenesulfonamide

Mass spectrum: Found: MH⁺ 426

H.p.l.c. (1) 3.11 min

### Example 114

### N-{4-[(3S)-3-({[(E)-2-(5-Chloro-2-thienyl)ethenyl]sulfonyl}amino)-2-oxopyrrolidinyl]-3-fluorophenyl}acetamide

Mass spectrum: Found: MH⁺ 458

H.p.l.c. (1) Rt 2.96 min

### Example 115

### N-{4-[(3S)-3-({[(E)-2-(5-Chloro-2-thienyl)ethenyl]sulfonyl}amino)-2-oxopyrrolidinyl]-3-fluorophenyl}propanamide

Mass spectrum: Found: MH⁺ 472

H.p.l.c. (1) Rt 3.09 min

### Example 116

### N-{4-[(3S)-3-({[(E)-2-(5-Chlorothien-2-yl)ethenyl]sulfonyl}amino)-2-oxopyrrolidin-1yl]-3-fluorophenyl}-2-methylpropanamide

Mass spectrum: Found: MH⁺ 486

H.p.l.c. (1) Rt 3.20 min

### Example 117

### N-[4-((3S)-3-{[(6-Chloro-1-benzothien-2-yl)sulfonyl]amino}-2-oxopyrrolidinyl)-3-fluorophenyl]acetamide

Mass spectrum: Found: MH⁺ 482

H.p.l.c. (1) Rt 3.16 min

### Example 118

### N-[4-((3S)-3-{[(6-Chloro-1-benzothien-2-yl)sulfonyl]amino}-2-oxopyrrolidinyl)-3-fluorophenyl]propanamide

Mass spectrum: Found: MH⁺ 496

H.p.l.c. (1) Rt 3.28 min

### Example 119

### N-[4-((3S)-3-{[(6-Chloro-1-benzothien-2-yl)sulfonyl]amino}-2-oxopyrrolidinyl)-3-fluorophenyl]-2-methylpropanamide

Mass spectrum: Found: MH⁺ 510

H.p.l.c. (1) Rt 3.36 min

### Example 120

### (E)-2-(5-Chlorothien-2-yl)-N-((3S)-1-{2-fluoro-4-[formyl(isopropyl)amino]phenyl}-2-oxopyrrolidin-3-yl)ethenesulfonamide

Mass spectrum: Found: MH⁻ 484

H.p.l.c. (1) Rt 3.10 min

### Example 121

### 6-Chloro-N-((3S)-1-{2-fluoro-4-[formyl(isopropyl)amino]phenyl}-2-oxopyrrolidin-3-yl)-1-benzothiophene-2-sulfonamide

Mass spectrum: Found: MH⁻ 508

H.p.l.c. (1) Rt 3.27 min

### Example 122

### 6-Chloro-N-{(3S)-1-[2-fluoro-4-(1H-imidazol-1-yl)phenyl]-2-oxopyrrolidin-3-yl}naphthalene-2-sulfonamide

To a solution of Intermediate 106 (0.05 g) in DCM (5 ml) was added triflouroacetic acid (0.5 ml). After stirring for 2 h the reaction mixture was concentrated under reduced pressure to give (3S)-3-amino-1-[2-fluoro-4-(1H-imidazol-1-yl)phenyl]pyrrolidin-2-one (0.071 g) as an oil. Acetonitrile (5 ml) was added followed by N,N-diisopropylethylamine (84.8 μl) and 6-chloro-napthyl-2-sulfonyl chloride¹ (0.036 g). After stirring at room temperature for 18 h, the reaction mixture was concentrated under reduced and partitioned between DCM and saturated sodium hydrogen carbonate. The organic phase was dried (hydrophobic frit) and loaded onto a SPE (Silica, eluting with DCM, diethyl ether, ethyl acetate and acetone) to give the title compound (0.030 g) as a white solid.

Mass spectrum: Found: MH⁺ 485

H.p.l.c. (1) Rt 2.79 min

Using similar chemistry, the following were prepared:

### Example 123

### 6-Chloro-N-[(3S)-1-(2,4-dichlorophenyl)-2-oxopyrrolidinyl]-2-naphthalenesulfonamide

Mass spectrum: Found: MH⁺ 473

H.p.l.c. (1) Rt 3.67 min

### Example 124

### N-[(3S)-1-(4-tert-Butyl-1,3-thiazol-2-yl-2-oxopyrrolidinyl]-6-chloro-2-naphthalenesulfonamide

Mass spectrum: Found: MH⁺ 464

H.p.l.c. (1) Rt 3.94 min

### Example 125

### N-[(3S)-1-(4-tert-butylphenyl)-2-oxopyrrolidinyl]-6-chloro-2-naphthalenesulfonamide

Mass spectrum: Found: MH⁺ 457

H.p.l.c. (1) Rt 3.90 min

### Example 126

### (1E)-2-(5-chlorothien-2-yl)-N-[(3S)-2-oxo-1-pyrazin-2-ylpyrrolidin-3-yl]prop-1-ene-1-sulfonamide

Mass spectrum: Found: MH⁺ 399

H.p.l.c. (1) Rt 3.08 min

### Example 127

### 6-Chloro-N-[(3S)-2-oxo-1-(1,3-thiazol-2-yl)pyrrolidinyl]-2-naphthalenesulfonamide

Mass spectrum: Found: MH⁺ 408

H.p.l.c. (1) Rt 3.31 min

### Example 128

### 6-Chloro-N-{(3S)-1-[2-fluoro-4-(4-methyl-1H-imidazol-1-yl)phenyl]-2-oxopyrrolidinyl}-2-naphthalenesulfonamide

Mass spectrum: Found: MH⁺ 499

H.p.l.c. (1) Rt 2.73 min

### Example 129

### 6-Chloro-N-{(3S)-1-[2-fluoro-4-(1H-pyrazol-1-yl)phenyl]-2-oxopyrrolidinyl}-2-naphthalenesulfonamide

Mass spectrum: Found: MH⁺ 485

H.p.l.c. (1) Rt 3.37 min

### Example 130

### N-[(3S)-1-(5-Bromo-1,3-thiazol-2-yl)-2-oxopyrrolidinyl]-2-(5-chloro-2-thienyl)ethanesulfonamide

Mass spectrum: Found: MH⁺ 471.5

H.p.l.c. (1) Rt 3.63 min

### Example 131

### 6-Chloro-N-[(3S)-1-(pyrazin-2-yl)-2-oxopyrrolidin-3-yl]-1-benzothiphene-2-sulfonamide

Mass spectrum: Found: MH⁺ 409

H.p.l.c. (1) Rt 3.26 min

### Example 132

### 2-(5-Chlorothien-2-yl)-N-[(3S)-1-(5-iodopyridin-2-yl)-2-oxopyrrolidin-3-yl]ethane-1-sulfonamide

Mass spectrum: Found: MH⁺ 512

H.p.l.c. (1) Rt 3.59 min

### Example 133

### 4-[(3S)-3-((2-Amino-2-oxoethyl){[(E)-2-(5-chlorothien-2-yl)ethenyl]sulfonyl}amino)-2-oxopyrrolidin-1yl]-3-fluorobenzamide

A solution of Example 107 (0.10 g) in dry acetonitrile (5 ml) was treated with cesium carbonate (0.092 g) and bromoacetamide (0.039 g) and stirred at ambient temperature for 18 h. Solvent was removed under reduced pressure, partitioning the residue between ethyl acatate and saturated sodium bicarbonate solution. The separated organic layer was washed with water, dried (over magnesium sulphate) and concentrated under reduced pressure to offer crude material which was purified using mass directed preparative h.p.l.c. to give the title compound (0.038 g) as a white powder.

Mass spectrum: Found: MH⁺ 501

H.p.l.c. (1) Rt 2.66 min

Using similar chemistry, the following was prepared:

### Example 134

### 4-[(3S)-3-((2-Amino-2-oxoethyl){[(E)-2-(5-chlorothien-2-yl)ethenyl]sulfonyl}amino)-2-oxopyrrolidin-1-yl]-3-fluoro-N,N-dimethylbenzamide

Mass spectrum: Found: MH⁻ 529

H.p.l.c. (1) Rt 2.76 min

### Example 135

### (E)-2-(5-Chlorothien-2-yl)-N-{(3S)-1-[2-fluoro-4-(1-hydroxyethyl)phenyl]-2-oxopyrrolidin-3-yl}ethenesulfonamide

Example 112 (0.163 g) suspended in dry methanol (5 ml) was treated with sodium borohydride (0.028 g) and the mixture stirred at ambient temperature for 90 min under nitrogen. The reaction was quenched with 3 drops water and concentrated under reduced pressure, partitioning the residue between DCM and water. The separated organic layer was dried (hydrophobic frites) and concentrated under reduced pressure to give the title compound (0.149 g) as a beige foamy solid.

Mass spectrum: Found: MH⁺ 445

H.p.l.c. (1) Rt 3.00 min

### Example 136

### (1E-2-(5-Chlorothien-2-yl)-N-[(3S)-1-(5-iodopyridin-2-yl)-2-oxopyrrolidin-3-yl]prop-1-ene-1-sulfonamide

Mass spectrum: Found: MH⁺ 524

H.p.l.c. (1) Rt 3.65 min

Using similar chemistry to Example 89, the following were prepared:

### Example 137

### (1E)-2-(5-Chlorothien-2-yl)-N-((3S)-1-{2-fluoro-4-[(methylsulfonyl)amino]phenyl}-2-oxopyrrolidin-3-yl)prop-1-ene-1-sulfonamide

Mass spectrum: Found: MH⁺ 508

H.p.l.c. (1) Rt 3.10 min

### Example 138

### (E)-N-[(3S)-1-(4-Acetylphenyl)-2-oxopyrrolidin-3-yl]-2-(5-chlorothien-2-yl)ethenesulfonamide

Mass spectrum: Found: MH⁺ 424

H.p.l.c. (1) Rt 3.16

### Example 139

### (A) 2-({(3S)-1-[2′-(Aminosulfonyl)-3-fluoro-1,1′-biphenyl-4-yl]-2-oxopyrrolidin-3-yl}{[(1E)-2-(5-chlorothien-2-yl)prop-1-enyl]sulfonyl}amino)acetamide and (B) 2-({(3S)-1-[2′-(Aminosulfonyl)-3-fluoro-1,1′-biphenyl-4-yl]-2-oxopyrrolidin-3-yl}{[(1Z)-2-(5-chlorothien-2-yl)prop-1-enyl]sulfonyl}amino)acetamide

Using Example 16 and 2-bromoacetamide, and the synthetic procedure described to prepare Example 40, the title compounds were prepared.

### Example A

Mass spectrum: Found: MH⁺ 627

H.p.l.c. (1) Rt 3.13 min

### Example B

Mass spectrum: Found: MH⁺ 627

H.p.l.c. (1) Rt 3.09 min

Using similar chemistry, the following was also prepared:

### Example 140

### 2-{(6-Chloro-benzo[b]thiophene-2-sulphonyl)-[(S)-1-(3-fluoro-2′-sulfamoyl-biphenyl-4-yl)-2-oxo-pyrrolidin-3-yl]-amino}-acetamide Formate

Mass spectrum: Found: MH⁺ 637

H.p.l.c. (1) Rt 3.21 min

### Example 141

### 2-(5-Chlorothien-2-yl)-N-[(3S)-1-(4-{2-[(dimethylamino)methyl]-1H-imidazol-1yl}-2-fluorophenyl)-2-oxopyrrolidin-3-yl]ethanesulfonamide

Using Intermediate 16 and Intermediate 146, and the synthetic procedure described to prepare Example 1, the title compound was prepared.

Mass spectrum: Found: MH⁺ 526

H.p.l.c. (1) Rt 2.36 min

### Example 142

### 2-Amino-N-[(1-{4-[(3S)-3-({[(1E)-2-(5-chlorothien-2-yl)prop-1-enyl]sulfonyl}amino)-2-oxopyrrolidin-1-yl]-3-fluorophenyl}-1H-imidazol-2-yl)methyl]-N,N-dimethyl-2-oxoethanaminium formate

Using Example 14 and 2-bromoacetamide, and the synthetic procedure described to prepare Example 40, the title compound was prepared.

Mass spectrum: Found: MH⁺ 595

H.p.l.c. (1) Rt 2.44 min

Using similar chemistry, the following were also prepared:

### Example 143

### 2-Amino-N-[(1-{4-[(3S)-3-({[2-(5-chlorothien-2-yl)ethyl]sulfonyl}amino)-2-oxopyrrolidin-1-yl]-3-fluorophenyl}-1H-imidazol-2-yl)methyl]-N,N-dimethyl-2-oxoethanaminium Formate

Mass spectrum: Found: MH⁺ 583

H.p.l.c. (1) Rt 2.36 min

### Example 144

### 2-Amino-N-({1-[4-((3S)-3-{[(6-chloro-1-benzothien-2-yl)sulfonyl]amino}-2-oxopyrrolidin-1-yl)-3-fluorophenyl]-1 H-imidazol-2-yl}methyl)-N,N-dimethyl-2-oxoethanaminium Formate

Mass spectrum: Found: MH⁺ 605

H.p.l.c. (1) Rt 2.52 min

Intermediate 1

tert-Butyl (2S)-1-({[4-(dimethylamino)phenyl]amino}carbonyl)-3-hydroxypropylcarbamate

To a solution of 4-(N,N-dimethylamino)aniline (0.061 g) in anhydrous DCM (2 ml) at room temperature under nitrogen was treated with a solution of trimethyaluminium (2.0M solution in hexane; 0.224 ml) dropwise over ca. 10 min. The resultant solution was stirred at room temperature for a further 15 min before a solution of (S)-N-(tert-butoxycarbonyl)homoserine (0.060 g) in anhydrous DCM (1 ml) was added slowly. The mixture was quenched after stirring at room temperature for 18 h by addition of 0.5N aqueous hydrochloric acid. The separated organic layer was washed with brine, filtered through hydrophobic frits and evaporated under a stream of nitrogen. The resultant residue was purified using a 10 g Redisep™ cartridge (silica, eluting with a gradient of 5% to 60% ethyl acetate:cyclohexane) to give the title compound (0.029 g).

Mass spectrum: Found: MH⁺ 338

Intermediate 2

tert-Butyl (3S)-1-[4-(dimethylamino)phenyl]-2-oxopyrrolidin-3-ylcarbamate

A solution of diisopropyl azodicarboxylate (0.023 g) in anhydrous THF (1 ml) at room temperature under nitrogen was treated with tri-n-butyl phoshphine (0.028 ml) and the solution stirred for 5 min. This solution was then added dropwise to a solution of tert-butyl (2S)-1-({[4-(dimethylamino)phenyl]amino}carbonyl)-3-hydroxypropylcarbamate (0.029 g) in anhydrous THF (1 ml) cooled to 0° C. under nitrogen. The resultant solution was allowed to warm to room temperature then stirred for a further 18 h. The reaction was evaporated under a stream of nitrogen. The residue was partitioned between saturated aqueous sodium bicarbonate solution (5 ml) and DCM (5 ml). The organic layer was separated, filtered through hydrophobic frits and evaporated under a stream of nitrogen. The resultant residue was purified using a 4 g Redisep™ cartridge (silica, eluting with a gradient of cyclohexane:ethyl acetate 3:1 increasing to 1:1 over 15 min) to give the title compound (0.026 g).

Mass spectrum: Found: MH⁺ 320

Intermediate 3

(3S)-3-Amino-1-[4-(dimethylamino)phenyl]pyrrolidin-2-one

tert-Butyl (3S)-1-[4-(dimethylamino)phenyl]-2-oxopyrrolidin-3-ylcarbamate (0.026 g) was treated with TFA-DCM (1:1, 1 ml) at room temperature and the solution aged for 1 h and then evaporated under a stream of nitrogen. The residue was re-dissolved in DCM/methanol and loaded onto a pre-equilibrated SCX SPE cartridge (1 g). The non-basic components were eluted with methanol and the required amine was eluted with 5% ammonia:methanol. The solvent was evaporated under reduced pressure to give the title compound (0.0074 g).

H.p.l.c. (1) Rt 2.38 min

Intermediate 4

tert-Butyl (2S)-1-({[3-fluoro-2′-(methylsulfonyl)-1,1′-biphenyl-4-yl]amino}carbonyl)-3-hydroxypropylcarbamate

A solution of 3-fluoro-2′-(methylsulfonyl)-1,1′-biphenyl-4-ylamine (0.318 g) in anhydrous DCM (3 ml) at room temperature under nitrogen was treated with solution of trimethyaluminium (2.0M solution in heptane; 0.6 ml) dropwise over ca. 10 min. The resultant solution was stirred at room temperature for a further 15 min before a solution of (S)-N-(tert-butoxycarbonyl)homoserine (0.200 g) in anhydrous DCM (3 ml) was added slowly. The mixture was quenched after stirring at room temperature for 18 h by addition of aqueous hydrochloric acid (1N, 4 ml). The separated organic layer was washed with brine, dried (over magnesium sulphate), and concentrated under reduced pressure. The residue was purified using a 35 g Redisep™ cartridge (silica, eluting with a gradient of cyclohexane:ethyl acetate 2:1 increasing to 1:2 over 20 min) to give the title compound (0.203 g) as a colourless glass.

Mass spectrum: Found: MH⁺ 466

Intermediate 5

tert-Butyl (3S)-1-[3-fluoro-2′-(methylsulfonyl)-1,1′-biphenyl-4-yl]-2-oxopyrrolidin-3-ylcarbamate

A solution of diisopropyl azodicarboxylate (0.128 g) in anhydrous THF (3 ml) at room temperature under nitrogen was treated with tri-n-butyl phoshphine (0.198 ml) and the solution stirred for 5min. This solution was then added dropwise to a solution of tert-butyl (2S)-1-({[3-fluoro-2′-(methylsulfonyl)-1,1′-biphenyl-4-yl]amino}carbonyl)-3-hydroxypropylcarbamate (0.200 g) in anhydrous THF (3 ml) cooled to 0° C. under nitrogen. The resultant solution was allowed to warn to room temperature and then stirred for a further 28 h. The reaction was concentrated under reduced pressure. The residue was partitioned between sodium bicarbonate solution and DCM. The organic layer was separated, washed with brine, then dried (over magnesium sulphate) and concentrated under reduced pressure to give the crude title compound (0.300 g) as a pale yellow gum.

Mass spectrum: Found: MH⁺ 449

A portion of this material was purified using SPE (silica, eluting with a gradient of 30-40% cyclohexane:ethyl acetate over 21 min) to give a sample of the pure title compound as a colourless gum.

Intermediate 6

(3S)-3-Amino-1-[3-fluoro-2′-(methylsulfonyl)-1,1′biphenyl-4-yl]pyrrolidin-2-one

Unpurified tert-butyl (3S)-1-[3-fluoro-2′-(methylsulfonyl)-1,1′-biphenyl-4-yl]-2-oxopyrrolidin-3-ylcarbamate (0.150 g) was treated with trifluoroacetic acid-DCM (1:1, 1 ml) at room temperature and the solution aged for 1 h and then concentrated under reduced pressure. The residue was re-dissolved in methanol (2 ml) and loaded onto a pre-equilibrated SCX SPE cartridge. The non-basic components were eluted with methanol and the required amine was eluted with 5% ammonia/methanol. The solvent was concentrated under reduced pressure to give the title compound (0.042 g) as a white solid.

Mass spectrum: Found: MH⁺ 348

Intermediate 7

1-Bromo-2-(methylsulfonyl)benzene

2-Bromothioanisole (6.0 g), was dissolved in DCM (234 ml) and stirred under nitrogen at −5° C. in an ice/salt bath. 3-Chloroperoxybenzoic acid (22.8 g) was added portionwise, maintaining the temperature between −5 and 0° C. When the addition was complete, the reaction mixture was warmed to ambient temperature and stirred for 4.5 h. The reaction mixture was washed with saturated aqueous sodium sulphite solution, saturated aqueous sodium bicarbonate solution, dried (over magnesium sulphate), filtered and concentrated under reduced pressure. The residue was triturated with 40-60 petroleum ether, filtered and dried under vacuum at 30° C. to give the title compound (7.58 g) as a white solid.

Mass spectrum: Found: MH⁺ 237

Intermediate 8

tert-Butyl (1S)-3-hydroxy-1-{[(5-iodopyridin-2-yl)amino]carbonyl}propylcarbamate

To a solution of 2-amino-5-iodopyridine (20 g) in anhydrous DCM (150 ml) cooled to 0° C. under nitrogen, a solution of trimethylaluminium (2M in hexanes, 45.15 ml) was added slowly. The mixture was stirred for a further 1 h (warmed to 10° C.). A cooled solution (0° C.) of (S)-N-(tert)butoxycarbonyl)homoserine (15.1 g) in anhydrous DCM (150 ml) was added dropwise. The resulting solution was allowed to warm to ambient temperature over 1 h. After stirring for a further 26 h, the reaction mixture was diluted with DCM (200 ml) and sodium fluoride (15.2 g) was added. The mixture was cooled to 0° C. and water (4.87 ml) was added dropwise. After stirring vigorously for a further 10 min at 0° C., and 30 min at ambient temperature, the mixture was filtered through Celite™ and washed with DCM. The combined organic solutions were concentrated under reduced pressure to give the crude product which was purified by reverse phase Biotage™ chromatography, (silica, eluting with 10% to 100% acetonitrile:water) to give the title compound (12.7 g) as a white solid.

¹H NMR in CDCl₃: δ9.10(1H, br.s), 8.17(1H, d), 8.05(1H, br.d), 7.96(1H, dd), 5.75(1H, br.d), 4.55(1H, br.s), 3.85(2H, m), 2.15-1.90(2H, 2×m), 1.45(9H, s) ppm.

Using similar chemistry, the following was prepared:

Intermediate 9

tert-Butyl(1S)-1-{[(2-fluoro-4-iodophenyl)amino]carbonyl}-3-hydroxypropylcarbamate

Mass spectrum: Found: MH⁺ 439

Intermediate 10

tert-Butyl (3S)-1-5-iodopyridin-2-yl)-2-oxopyrrolidin-3-ylcarbamate

To a solution of di-tert-butyl azodicarboxylate (8.9 g) in anhydrous THF (50 ml) at 0° C. under nitrogen, tri-n-butylphosphine (9.61 ml) followed by a solution of Intermediate 8 (12.5 g) in anhydrous THF (100 ml) was added. The reaction mixture was stirred at 0° C. for a further 1 h and then at ambient temperature for a further 16 h. The reaction mixture was concentrated under reduced pressure and treated with DCM and saturated aqueous sodium bicarbonate solution. The organic layer was separated, washed with brine, dried (over magnesium sulphate) and filtered. The organic phase was concentrated under reduced pressure to give the crude product which was purified by flash column chromatography (silica, eluting with cyclohexane:ethyl acetate, 5:2 to 2:1) to give the title compound (12.5 g) as a white solid.

¹H NMR in CDCl₃: δ8.55δ(1H, d), 8.25(1H, d), 7.95(1H, dd), 5.1.5(1H, m), 4.55(1H, br.m), 4.20-3.80(2H, 2×m), 2.73-1.97(2H, 2×m), 1.60(9H, s) ppm.

Using similar chemistry, the following was prepared:

Intermediate 11

tert-Butyl (3S)-1-(2-fluoro-4-iodophenyl)-2-oxopyrrolidin-3-ylcarbamate

Mass spectrum: Found: MH⁺ 421

Intermediate 12

tert-But (3S)-2-oxo-1-[5-(4,4,5,5-tetramethyl-1,3,2-dioxaborolan-2-yl)pyridin-2-yl]pyrrolidin-3-ylcarbamate

Intermediate 8 (1.0 g) was dissolved in anhydrous DMF (12 ml) and stirred under nitrogen at ambient temperature. Potassium acetate (0.733 g) and bis(pinacolato)diboron (0.067 g) followed by 1,1′-bis(diphenylphosphino)ferrocene dichloropalladium (II) (0.09 g) were added and the reaction mixture was stirred and heated at 80° C. under nitrogen for 5.5 h. The reaction mixture was cooled to ambient temperature and diluted with ethyl acetate. The organic layer was washed with saturated brine, water and then dried (over magnesium sulphate), filtered and concentrated under reduced-pressure. The residue was dried under high vacuum to give the title compound as a brown solid (1.42 g).

Tlc (SiO₂, cyclohexane:ether, 1:3), Rf 0.40.

Intermediate 13

tert-Butyl (3S)-1-{5-[2-(methylsulfonyl)phenyl]pyridin-2-yl}-2-oxopyrrolidin-3-ylcarbamate

Intermediate 12 (1.42 g) was dissolved in anhydrous DME (40 ml) and stirred under nitrogen at ambient temperature. Intermediate 7 (1.0 g) was added, followed by potassium carbonate (2.43 g) and 1,1′-bis(diphenylphosphino)ferrocene dichloropalladium (II) (0.213 g). The dark brown reaction mixture was stirred at 80° C. for 22 h. The reaction mixture cooled to ambient temperature and concentrated under reduced pressure. The residue was partitioned between ethyl acetate and water. The aqueous layer was separated and re-extracted with ethyl acetate. The combined organic layers were dried (over magnesium sulphate), filtered and concentrated under reduced pressure. The residue was purified using Biotage™ chromatography (silica, eluting with cyclohexane:ether 1:7) to give the title compound (0.49 g) as a yellow foam.

Mass spectrum: Found: MH⁺ 432

Intermediate 14

(3S)-3-Amino-1-{5-[2-(methylsulfonyl)phenyl]pyridin-2-yl}pyrrolidin-2-one

Intermediate 13 (0.49 g) was dissolved in anhydrous DCM (15 ml) and stirred in an ice bath under nitrogen. Trifluoroacetic acid (15 ml) was added slowly to the reaction mixture and then warmed to ambient temperature and stirred for 2.5 h. The reaction mixture was concentrated under reduced pressure. The residue was purified by SPE (silica, eluting with methanol to 2-5% aqueous ammonia in methanol) to give the title compound (0.310 g) as a white foam.

Mass spectrum: Found: MH⁺ 332

Intermediate 15

tert-Butyl (3S)-1-(4-{2-[(dimethylamino)methyl]-1H-imidazol-1-yl}-2-fluorophenyl)-2-oxopyrrolidin-3-ylcarbamate

A suspension of Intermediate 11 (2.10 g), 2-(N,N-dimethylamino)methyl imidazole (0.682 g), anhydrous potassium carbonate (0.737 g), 8-hydroxyquinoline (0.047 g) in anhydrous dimethylsulphoxide (5 ml) was stirred under nitrogen at ambient temperature. Copper (I) iodide (0.045 g) was added and the reaction mixture was heated to 122° C. and stirred for 17 h. The reaction mixture was cooled to ambient temperature. 17% Aqueous ammonium hydroxide was added and the mixture was stirred for 1 h. The reaction mixture was extracted with ethyl acetate. The combined organic layers were washed with 17% aqueous ammonium hydroxide, dried (over magnesium sulphate), filtered and concentrated under reduced pressure. The residue was purified using Biotage™ chromatography (silica, eluting with DCM:methanol 9:1) to give the title compound (1.75 g) as a dark brown oil.

Tlc (SiO₂, CHCl₃:MeOH:H₂O, 65:30:5) R_{f} 0.7

Intermediate 16

(3S)-3-Amino-1-(4-{2-[(dimethylamino)methyl]-1H-imidazol-1-yl}-2-fluorophenyl)pyrrolidin-2-one

Intermediate 15 (1.75 g) was dissolved in DCM (11 ml) and stirred under nitrogen at ambient temperature. Trifluoroacetic acid (11 ml) was added and the reaction mixture was stirred at ambient temperature for 1 h. The reaction mixture was concentrated under reduced pressure. The residue was purified using SPE (silica, eluting with methanol: aqueous ammonia 50:1, and then 19:1) and then using Biotage™ chromatography (silica, eluting with DCM:methanol 9:1) to give the title compound (0.679 g) as a brown oil.

Tlc (SiO₂, CHCl₃:MeOH:H₂O, 65:30:5) R_{f} 0.40

Intermediate 17

Di(tert-butyl) (2-bromophenyl)sulfonylimidodicarbonate

1-Bromobenzenesulphonamide (15.40 g) was partially dissolved in anhydrous acetonitrile (300 ml) and stirred at ambient temperature under nitrogen. Di-tert-butyl dicarbonate (68 g) was added in portions followed by 4-dimethylaminopyridine (3.30 g). The reaction mixture was stirred at ambient temperature for 2 h. Di-tert-butyl dicarbonate (34.0 g) was added in portions followed by 4-dimethylaminopyridine (2.55 g). The reaction mixture was stirred at ambient temperature for 18 h. The reaction mixture was concentrated under reduced pressure and the residue was purified using flash vacuum chromatography (silica, eluting with cyclohexane:ether, 3:1) to give the title compound (17.3 g) as a yellow solid.

Tlc (SiO₂, cyclohexane:ether, 3:1), R_{f} 0.32.

Intermediate 18

tert-Butyl (3S)-1-[2-fluoro-4-(4,4,5,5-tetramethyl-1,3,2-dioxaborolan-2-yl)phenyl]-2-oxopyrrolidin-3-ylcarbamate

Intermediate 11 (2.0 g) was dissolved in anhydrous DMF (23 ml) and stirred under nitrogen at ambient temperature. Potassium acetate (1.41 g) and bis(pinacolato)diboron (1.29 g) followed by 1,1′-bis(diphenylphosphino)ferrocene dichloropalladium (II) (0.173 g) were added and the reaction mixture was stirred and heated at 80° C. under nitrogen for 5 h. The reaction mixture was cooled to ambient temperature and diluted with ethyl acetate. The organic layer was washed with saturated brine, water and then dried (over magnesium sulphate), filtered and concentrated under reduced-pressure. The residue was dried under high vacuum to give the title compound (2.8 g) as a brown solid.

Tlc (SiO₂, cyclohexane:ether, 1:3), R_{f} 0.30.

Intermediate 19

Di(tert-butyl) (4′-{(3S)-3-[(tert-butoxycarbonyl)amino]-2-oxopyrrolidin-1-yl}-3′-fluoro-1,1′-biphenyl-2-yl)sulfonylimidodicarbonate

Intermediate 18 (2.5 g) was dissolved in anhydrous DME (70 ml) and stirred under nitrogen at ambient temperature. Intermediate 17 (2.58 g) was added, followed by potassium carbonate (4.11 g) and 1,1′-bis(diphenylphosphino)ferrocene dichloropalladium (II) (0.36 g). The dark brown reaction mixture was stirred at 80° C. for 18 h. The reaction mixture was cooled to ambient temperature and concentrated under reduced pressure. The residue was partitioned between ethyl acetate and water. The aqueous layer was separated and re-extracted with ethyl acetate twice. The combined organic layers were dried (over magnesium sulphate), filtered and concentrated under reduced-pressure. The residue was purified using Biotage™ chromatography (silica, eluting with cyclohexane:ether 1:3) to give the title compound (0.53 g) as a cream froth.

Mass spectrum: Found: MH⁺ 651

Intermediate 20

Di(tert-butyl) {4′-[(3S)-3-amino-2-oxopyrrolidin-1-yl]-3′-fluoro-1,1′-biphenyl-2-yl}sulfonylimidodicarbonate

Intermediate 19 (0.53 g) was dissolved in anhydrous DCM (5 ml) and stirred under nitrogen at ambient temperature. Trifluoroacetic acid (5 ml) was added and the reaction mixture was stirred at ambient temperature for 2 h and then concentrated under reduced pressure. The residue was purified using SPE (silica, eluting with methanol, methanol:aqueous ammonia 50:1, 19:1) to give the title compound (0.287 g) as a cream froth.

Mass spectrum: Found: MH⁺ 350

Intermediate 21

tert-Butyl (3S)-1-[5-(2-nitrophenyl)pyridin-2-yl]-2-oxopyrrolidin-3-ylcarbamate

A solution of crude Intermediate 12, (0.128 g) in dry ethyleneglycol dimethylether (8 ml) was treated sequentially with 1-iodo-2-nitrobenzene (0.095 g), potassium carbonate (0.219 g) and 1,1′bis(diphenylphosphino) ferrocene palladium dichloride (0.018 g). The mixture was heated to 80° C. under nitrogen for 6 h. The resulting black suspension was cooled to room temperature and diluted with ethyl acetate washing with saturated sodium chloride solution and water. The separated organic layer was dried (over magnesium sulphate) and concentrated under reduced pressure to give a crude brown gum. This gum like solid was purified using SPE (silica, eluting with cyclohexane: ethyl acetate 19:1 to 1:1) to give the title compound (0.095 g) as a pale yellow gum

Mass spectrum: Found: MH⁺ 399

Using similar chemistry, the following was prepared:

Intermediate 22

tert-Butyl(3S)-1-(3-fluoro-2′-nitro-1,1′-biphenyl-4-yl)-2-oxopyrrolidin-3-ylcarbamate

Mass spectrum: Found: MH⁺ 416

Intermediate 23

(3S)-3-Amino-1-[5-(2-nitrophenyl)pyridin-2-yl]pyrrolidin-2-one Hydrochloride

Intermediate 21 (0.095 g) was stirred in 4N hydrochloric acid/dioxane (10 ml) at 0° C. for 1 h, allowing to warm up to room temperature over 18 h. The resulting pale yellow suspension was concentrated under reduced pressure to give the title compound (0.081 g) as a yellow powder.

Mass spectrum: Found: MH⁺ 299

Using similar chemistry and Intermediate 22, the following was prepared:

Intermediate 24

(3S)-3-Amino-1-(3-fluoro-2′-nitro- 1,1′-biphenyl-4-yl)pyrrolidin-2-one Hydrochloride

Mass spectrum: Found: MH⁺ 316

Intermediate 25

tert-Butyl(3S)-2-oxo-1-(5-phenylpyridin-2-yl)pyrrolidin-3-ylcarbamate

A solution of Intermediate 10 (0.15 g) in ethyleneglycol dimethylether: water (15 ml, 2:1) was treated, sequentially with sodium carbonate (0.103 g), phenyl boronic acid (0.054 g) and tetrakistriphenylphospinepalladium(0) (0.015 g). The pale yellow solution was heated to 80° C. for 5 h. The cooled reaction mixture was concentrated under reduced pressure, partitioning the residue between diethyl ether and water. The separated organic layer was dried (over magnesium sulphate) and concentrated under reduced pressure to give a crude orange residue which was purified using SPE (silica, eluting with cyclohexane: ethyl acetate 4:1) to give the title compound (0.10 g) as a white powder.

Mass spectrum: Found: MH⁺ 354

Intermediate 26

1-Bromo-2-isopropoxybenzene

A solution of 2-bromophenol (1.0 g) in dry N′N-dimethylformamide (10 ml) was treated with potassium carbonate (1.2 g) followed by 2-bromopropane (0.711 g). The mixture was heated to 60° C. for 18 h. The cooled reaction was concentrated under reduced pressure and the residue partitioned between diethyl ether and 1N sodium hydroxide, washing the separated organic layer with saturated sodium chloride solution and water. The separated organic layer was dried (over magnesium sulphate) and concentrated under reduced pressure to give the title compound (1.18 g) as a colourless oil.

¹H NMR in CDCl₃: δ1.38 (d, 6H), 4.55 (septet, 1H), 6.82 (t, 1H), 6.93 (d, 1H), 7.24 (t, 1H), 7.53 (d, 1H)ppm.

Intermediate 27

tert-Butyl (2-Bromophenyl)sulphonylcarbamate

Aqueous ammonia solution (50 ml) was added to a stirred solution of 2-bromobenzenesulphonyl chloride (5.0 g) in tetrahydrofuran (100 ml) at 5° C. The mixture was stirred for 20 min and then concentrated under reduced pressure, triturating the residue with water. The solid was collected by filtration and suspended in DCM (100 ml). 4-(Dimethylamino)pyridine (0.25 g) and triethylamine(3.2 ml) were added , followed by di-tert-butyl dicarbonate (5.8 g) and the solution was stirred at ambient temperature for 1 h. The solution was washed with 1N hydrochloric acid, water and dried (over sodium sulphate). The solvent was removed under reduced pressure to give the title compound (5.8 g) as a white powder.

H.p.l.c. (1) Rt 3.08 min

Intermediate 28

tert-Butyl(2-bromophenyl)sulfonyl{[2-(trimethylsilyl)ethoxy]methyl}carbamate

A solution of Intermediate 27 (1.0 g) in dry THF (15 ml), at 0° C., was treated with sodium hydride (0.14 g) portionwise. The mixture was stirred for 45 min before a solution of 2-(trimethylsilyl)ethoxymethyl chloride (0.63 ml) in dry THF (10 ml) was added dropwise. The reaction was allowed to warm up to room temperature and stirred for 18 h. The resulting white suspension was concentrated under reduced pressure, partitioning the residue between diethyl ether and water. The separated organic layer was washed with saturated sodium chloride, dried (over magnesium sulphate) and concentrated under reduced pressure to give crude material as a pale yellow oil. This was purified using SPE (silica, eluting with cyclohexane:ethylacetate 20:1 and 4:1) to give the title compound (1.29 g) as a pale yellow oil.

Mass spectrum: Found: MH⁺ 485

Intermediate 29

N-(2-Bromophenyl)-N-methylmethanesulphonamide

A solution of N-(2-bromophenyl)methanesulphonamide (0.2 g) in dry acetonitrile (5 ml), at 0° C., was treated with potassium carbonate (0.167 g) followed by iodomethane (0.34 g). The mixture was allowed to warm up to room temperature and stirred for 18 h. Solvent was removed under reduced pressure and the residue partitioned between DCM and water. The organic layer was dried through hydrophobic frits and concentrated under reduced pressure. The residue was purified using SPE (silica, eluting with cyclohexane:ethylacetate 20:1 to 2:1) to give the title compound (0.19 g) as white solid.

Mass spectrum: Found: MH⁺ 266

Intermediate 30

tert-Butyl(2-bromophenyl)sulphonyl(methyl)carbamate

A solution of Intermediate 27 (1.0 g) in dry THF (30 ml), at 0° C., was treated with sodium hydride (0.14 g) portionwise. The mixture was stirred for 45 min before iodomethane (1.27 g) was added slowly. The mixture was allowed to warm up to room temperature and stirred for 18 h. Solvent was removed under reduced pressure, partitioning the residue between ethyl acetate and water. The separated organic layer was washed with saturated sodium chloride, dried (over magnesium sulphate) and concentrated under reduced pressure to give crude material. This was purified using SPE (silica, eluting with cyclohexane:ethylacetate 20:1 to 2:1) to give the title compound (0.56 g) as a white solid.

Mass spectrum: Found MNH₄⁺ 369

A by-product from this reaction was:

Intermediate 31

2-Bromo-N,N-dimethylbenzene sulphonamide

Mass spectrum: Found: MH⁺ 266

Intermediate 32

N-(2-Bromophenyl)-N-{[2-(trimethylsilyl)ethoxy]methyl}methanesulfonamide

Using N-(2-bromophenyl)methanesulphonamide and the synthetic procedure described for Intermediate 28, the title compound was prepared.

Mass spectrum: Found MNH₄⁺ 399

Intermediate 33

1-Bromo-2-tert-butylbenzene

Bromine (0.25 ml) was added dropwise to a cooled flask of phosphorus tribromide (0.47 ml). To this was added 2-tert-butyl phenol (3 g) and the mixture heated to 230° C. for 2.5 h. The cooled reaction was partitioned between diethyl ether and 10% aqueous sodium thiosulphate solution. The separated organic layer was washed with 2N potassium hydroxide, dried (over magnesium sulphate) and concentrated under reduced pressure to give a crude orange oil. The residue was purified using Biotage™ chromatography (silica, eluting with cyclohexane:ethylacetate 19:1) to give the title compound (0.54 g) as a colourless oil.

¹H NMR in CDCl₃: δ1.50(s, 9H), 7.02(t, 1H), 7.23(t, 1H), 7.42(d, 1H), 7.59(d, 1H) ppm.

Intermediate 34

tert-Butyl(3S)-1-(3-fluoro-2′-nitro-1,1′-biphenyl-4-yl)-2-oxopyrrolidin-3-ylcarbamate

Using 1-iodo-2-nitrobenzene and the synthetic procedure described for Intermediate 21, the title compound was prepared.

Mass spectrum: Found: MH⁺ 416

Intermediate 35

tert-Butyl(4′-{(3S)-3-[(tert-butoxycarbonyl)amino]-2-oxopyrrolidin-1-yl}-3′-fluoro-1,1′-biphenyl-2-yl)sulfonyl(methyl)carbamate

Using Intermediate 29 and the synthetic procedure described for Intermediate 21, the title compound was prepared.

Mass spectrum: Found: MNH₄⁺ 399

Intermediate 36

tert-Butyl(4′-{(3S)-3-[(tert-butoxycarbonyl)amino]-2-oxopyrrolidin- 1-yl}-3′-fluoro-1,1′-biphenyl-2-yl)sulfonyl{[2-(trimethylsilyl)ethoxy]methyl}carbamate

Using Intermediate 28 and the synthetic procedure described for Intermediate 21, the title compound was prepared.

Mass spectrum: Found: MNH₄⁺ 697

Intermediate 37

tert-Butyl(3S)-1-{5-[2-((methylsulfonyl) {[2-(trimethylsilyl)ethoxy]methyl}amino)phenyl]pyridin-2-yl}-2-oxopyrrolidin-3-ylcarbamate

Using Intermediate 32 and the synthetic procedure described for Intermediate 21, the title compound was prepared.

Mass spectrum: Found: MH⁺ 577

Intermediate 38

tert-Butyl(3S)-1-[5-(2-tert-butylphenyl)pyridin-2-yl]-2-oxopyrrolidin-3-ylcarbamate

Using 1-iodo-2-nitrobenzene and the synthetic procedure described for Intermediate 21, the title compound was prepared.

Mass spectrum: Found: MH⁺ 410

Intermediate 39

tert-Butyl(3S)-2-oxo-1-{5-[2-(trifluoromethyl)phenyl]pyridin-2-yl}pyrrolidin-3-ylcarbamate

Using Intermediate 10 and the synthetic procedure described for Intermediate 25, the title compound was prepared.

Mass spectrum: Found: MH⁺ 422

Intermediate 40

tert-Butyl(3S)-1-(5-{2-[(dimethylamino)carbonyl]phenyl}pyridin-2-yl)-2-oxopyrrolidin-3-ylcarbamate

Using 2-iodo-N,N-dimethylbenzamide and the synthetic procedure described for Intermediate 21, the title compound was prepared.

Mass spectrum: Found: MH⁺ 425

Intermediate 41

tert-Butyl (3S)-1-[5-(2-cyanophenyl)pyridin-2-yl]-2-oxopyrrolidin-3-ylcarbamate

Using 2-bromobenzonitrile and the synthetic procedure described for Intermediate 21, the title compound was prepared.

Mass spectrum: Found: MH⁺ 379

Intermediate 42

tert-Butyl[2-(6-{(3S)-3-[(tert-butoxycarbonyl)amino]-2-oxopyrrolidin-1-yl}pyridin-3-yl)phenyl]sulfonyl{[2-(trimethylsilyl)ethoxy]methyl}carbamate

Using Intermediate 28 and the synthetic procedure described for Intermediate 21, the title compound was prepared.

Mass spectrum: Found: MH⁺ 663

Intermediate 43

tert-Butyl(3S)-1-(5-{2-[(dimethylamino)sulfonyl]phenyl}pyridin-2-yl)-2-oxopyrrolidin-3-ylcarbamate

Using Intermediate 31 and the synthetic procedure described for Intermediate 21, the title compound was prepared.

Mass spectrum: Found: MH⁺ 461

Intermediate 44

tert-Butyl[2-(6-{(3S)-3-[(tert-butoxycarbonyl)amino]-2-oxopyrrolidin-1-yl}pyridin-3-yl)phenyl]sulfonyl(methyl)carbamate

Using Intermediate 30 and the synthetic procedure described for Intermediate 21, the title compound was prepared.

Mass spectrum: Found: MH⁺ 547

Intermediate 45

tert-Butyl(3S)-1-(5-{2-[methyl(methylsulfonyl)amino]phenyl}pyridin-2-yl)-2-oxopyrrolidin-3-ylcarbamate

Using Intermediate 29 and the synthetic procedure described for Intermediate 21, the title compound was prepared.

Mass spectrum: Found: MH⁺ 461

Intermediate 46

tert-Butyl(3S)-1-[5-(2-isopropoxyphenyl)pyridin-2-yl]-2-oxopyrrolidin-3-ylcarbamate

Using Intermediate 26 and the synthetic procedure described for Intermediate 21, the title compound was prepared.

Mass spectrum: Found: MH⁺ 461

Intermediate 47

4′-[(3S)-3-Amino-2-oxopyrrolidin-1-yl]-3′-fluoro-N-methyl-1,1′-biphenyl-2-sulfonamide Hydrochloride

Using Intermediate 35 and the synthetic procedure described for Intermediate 24, the title compound was prepared.

Mass spectrum: Found: MH⁻ 362

Intermediate 48

4′-[(3S)-3-Amino-2-oxopyrrolidin-1-yl]-3′-fluoro-1,1′-biphenyl-2-sulfonamide Hydrochloride

Using Intermediate 36 and the synthetic procedure described for Intermediate 24, the title compound was prepared.

Mass spectrum: Found: MNH₄⁺ 367

Intermediate 49

N-(2-{6-[(3S)-3-Amino-2-oxopyrrolidin-1-yl]pyridin-3-yl}phenyl)methanesulfonamide Hydrochloride

Using Intermediate 37 and the synthetic procedure described for Intermediate 24, the title compound was prepared.

Mass spectrum: Found: MH⁺ 346

Intermediate 50

(3S)-3-Amino-1-[5-(2-tert-butylphenyl)pyridin-2-yl]pyrrolidin-2-one Hydrochloride

Using Intermediate 39 and the synthetic procedure described for Intermediate 24, the title compound was prepared.

Mass spectrum: Found: MH⁺ 346

Intermediate 51

(3S)-3-Amino-1-{5-[2-(trifluoromethyl)phenyl]pyridin-2-yl}pyrrolidin-2-one Hydrochloride

Using Intermediate 38 and the synthetic procedure described for Intermediate 23, the title compound was prepared.

Mass spectrum: Found: MH⁺ 322

Intermediate 52

2-{6-[(3S)-3-Amino-2-oxopyrrolidin-1-yl]pyridin-3-yl}-N,N-dimethylbenzamide Hydrochloride

Using Intermediate 40 and the synthetic procedure described for Intermediate 24, the title compound was prepared.

Mass spectrum: Found: MH⁺ 325

Intermediate 53

2-{6-[(3S)-3-Amino-2-oxopyrrolidin-1-yl]pyridin-3-yl}benzonitrile Hydrochloride

Using Intermediate 41 and the synthetic procedure described for Intermediate 24, the title compound was prepared.

Mass spectrum: Found: M^{H+} 279

Intermediate 54

2-{6-[(3S)-3-Amino-2-oxopyrrolidin-1-yl]pyridin-3-yl}benzenesulfonamide Hydrochloride

Using Intermediate 42 and the synthetic procedure described for Intermediate 24, the title compound was prepared.

Mass spectrum: Found: MH⁺ 333

Intermediate 55

2-{6-[(3S)-3-Amino-2-oxopyrrolidin-1-yl]pyridin-3-yl}-N,N-dimethylbenzenesulfonamide Hydrochloride

Using Intermediate 43 and the synthetic procedure described for Intermediate 24, the title compound was prepared.

Mass spectrum: Found: MH⁺ 361

Intermediate 56

2-{6-[(3S)-3-Amino-2-oxopyrrolidin-1-yl]pyridin-3-yl}-N-methylbenzenesulfonamide Hydrochloride

Using Intermediate 44 and the synthetic procedure described for Intermediate 24, the title compound was prepared.

Mass spectrum: Found: MH⁺ 347

Intermediate 57

N-(2-{6-[(3S)-3-Amino-2-oxopyrrolidin-1-yl]pyridin-3-yl}phenyl)-N-methylmethanesulfonamide Hydrochloride

Using Intermediate 45 and the synthetic procedure described for Intermediate 24, the title compound was prepared.

Mass spectrum: Found: MH⁺ 361

Intermediate 58

(3S)-3-Amino-1-{5-[2-(methylsulfonyl)phenyl]pyridin-2-yl}pyrrolidin-2-one Hydrochloride

Using Intermediate 13 and the synthetic procedure described for Intermediate 24, the title compound was prepared.

Mass spectrum: Found: MH⁺ 332

Intermediate 59

(3S)-3-Amino-1-{5-[2-(methylsulfonyl)phenyl]pyridin-2-yl}pyrrolidin-2-one Hydrochloride

Using Intermediate 46 and the synthetic procedure described for Intermediate 24, the title compound was prepared.

Mass spectrum: Found: MH⁺ 312

Intermediate 60

(3S)-3-Amino-1-(5-phenylpyridin-2-yl)pyrrolidin-2-one

Using Intermediate 25 and the synthetic procedure described for Intermediate 24, the title compound was prepared.

Mass spectrum: Found: MH⁺ 254

Intermediate 61

[(S)-1-(5-Bromo-thiazol-2-ylcarbamoyl)-3-hydroxy-propyl]-3-carbamic Acid-tert-butyl Ester

Using the chemistry described for Intermediate 8, the title compound was prepared.

Mass spectrum: Found: MH⁺ 379

Intermediate 62

[(S)-1-(bromo-thiazol-2-yl)-2-oxo-pyrrolidin-3-yl]-carbamic Acid-tert-butyl Ester

Using Intermediate 61 and the chemistry described for Intermediate 10, the title compound was prepared.

Mass spectrum: Found: MH⁺ 362

Intermediate 63

(S)-3-Amino-1-(5-bromo-thiazol-2-yl)-pyrrolidin-2-one Hydrochloride

Using Intermediate 62 and the chemistry described for Intermediate 23, the title compound was prepared.

Mass spectrum: Found: MH⁺ 262

Intermediate 64

6-Chloro-naphthalene-2-sulphonic Acid [(S)-1-(5-bromo-thiazol-2-yl)-2-oxo-pyrrolidin-3-yl]amide

Using Intermediate 63 and the chemistry described for Example 1, the title compound was prepared.

Mass spectrum: Found: MH⁺ 326

Intermediate 65

2-(5-Chlorothien-2-yl)-1,3-thiazole

To a mixture of 2-bromothiazole (0.325 g) and 5-chlorothiophene-2-boronic acid (0.322 g) in DME (10 ml) under nitrogen, a solution of sodium carbonate (0.546 g) in water (10 ml, followed by tris(dibenzylideneacetone)dipalladium(0)-chloroform adduct (0.05 g) and a solution of triphenylphosphine (0.052 g) in DME (10 ml) were added. The mixture was heated at 80° C. under nitrogen for 18 h and concentrated under reduced pressure. The resultant aqueous mixture was extracted with ethyl acetate, dried (over magnesium sulphate), filtered and concentrated under reduced pressure. The residue was partially purified using SPE (silica, cyclohexane:ethyl acetate 19:1 to 9:1) to give an impure sample of the titled intermediate. A portion (0.088 g) was further purified by preparative TLC (20 cm×20 cm, 1 mm thick Whatman PK6F SiO₂ 60 Å plate, eluting twice with cyclohexane:ethyl acetate 1:9) to give a pure batch of the title intermediate (0.058 g) as an off-white solid.

Mass spectrum: Found: MH⁺ 202

Intermediate Δ

N-Boc-N¹-(2-fluoro-4-bromophenyl)-L-homoserinamide

Using 2-fluoro-4-bromoaniline and the synthetic procedure described for Intermediate 8, the title compound was prepared.

Mass spectrum: Found: MH⁺ 391

Intermediate 67

tert-Butyl (3S)-1-(2-fluoro-4-bromophenyl)-2-oxopyrrolidin-3-ylcarbamate

Using Intermediate 66 and the synthetic procedure described for Intermediate 11, the title compound was prepared.

Mass spectrum: Found: MH⁺ 373

Intermediate 68

(3S)-3-Amino-1-(2-fluoro-4-iodophenyl)pyrrolidin-2-one Hydrochloride

4N Hydrochloric acid in dioxan (70 ml) was added to Intermediate 11 (5.23 g) and stirred at room temperature for 45 min. The mixture was concentrated under reduced pressure and the residue triturated in diethyl ether. The solid was filtered, washed and dried to give the title compound (3.79 g) as a white solid.

Mass spectrum: Found: MH⁺ 321

Using similar chemistry and Intermediate 67, the following was prepared:

Intermediate 69

(3S)-3-Amino-1-(2-fluoro-4-bromophenyl)pyrrolidin-2-one Hydrochloride

Mass spectrum: Found: MH⁺ 273

Intermediate 70

6-Chloro-N-[(3S)-1-(2-fluoro-4-iodophenyl)-2-oxopyrrolidin-3-yl]naphthalene-2-sulfonamide

Using Intermediate 68, and the synthetic procedure described to prepare Example 1, the title compound was prepared.

Mass spectrum: Found: MH⁺ 545

Using similar chemistry and Intermediate 69, the following was prepared:

Intermediate 71

N-[(3S)-1-(4-Bromo-2-fluorophenyl)-2-oxopyrrolidin-3yl]-6-chloronaphthalene-2-sulfonamide

Mass spectrum: Found: MH⁺ 497

Intermediate 72

tert-Butyl (3S)-1-(4-{2-[(dimethylamino)methyl]-1H-imidazol-1-yl}-2-fluorophenyl)-2-oxopyrrolidin-3-ylcarbamate

Copper (I) iodide was added to a mixture of Intermediate 11 (0.420 g), N-(1H-imidazol-2-ylmethyl)-N,N-dimethylamine (0.327 g) and potassium carbonate (0.345 g) in dimethylsulphoxide (2.5 ml) under nitrogen which had been degassed four times with a vacuum/nitrogen cycle. The mixture was again degassed four times using the same method then heated at 123° C. for 18 h. After cooling to 45° C., 17% ammonium hydroxide solution (5 ml) was added and the mixture stirred at room temperature for 1.5 h. The mixture was partitioned between ethyl acetate and water. The separated aqueous phase was extracted further with ethyl acetate, the combined organic extracts were washed with brine, then extracted into 10% citric acid. This solution was neutralised with 2N NaOH and extracted into DCM. The combined organic extracts were dried (over magnesium sulphate) and concentrated under reduced pressure to give the title compound (0.390 g) as a tan foam.

Mass spectrum: Found: MH⁺ 418

Intermediate 73

4-Propylpyridin-3-ylboronic Acid

A solution of 3-bromo-4-propylpyridine (4.6 g) in tetrahydrofuran (20 ml) was added dropwise to a solution of n-butyl lithium (15.4 ml, 1.62M in hexanes) in tetrahydrofuran (100 ml) at −95° C. under nitrogen. The solution was warmed to −78° C., stirred for 5 min and treated dropwise with triisopropyl borate (6.0 g) in tetrahydrofuran (10 ml). The resulting suspension was allowed to warm to room temperature, stirred for 30 min, and treated with water (200 ml). The mixture was neutralised with hydrochloric acid (2N, ca. 12 ml ) and extracted with diethyl ether. The dried (over magnesium sulphate) organic extracts was concentrated under reduced pressure to give the title compound (1.75 g) as a pale yellow solid.

Mass spectrum: Found: MH⁺ 166

Intermediate 74

tert-Butyl (3S)-1-[4-(2-chloropyridin-3-yl)-2-fluorophenyl]-2-oxopyrrolidin-3-ylcarbamate

A mixture of Intermediate 18 (0.084 g), 1,1′-bis(diphenylphosphino)ferrocene dichloro palladium(II) complex with DCM (0.016 mg), potassium acetate (0.138 g) and 2-chloro-3-bromopyridine (0.046 g) in degassed dimethoxyethane (5 ml) was heated at 80° C. overnight under nitrogen, then diluted with methanol and added to a SPE (SCX-2) column (eluting with methanol) to give the title compound (0.067 g) as an off white solid.

Mass spectrum: Found: MH⁺ 406

Intermediate 75

tert-Butyl (3S)-1-[4-(2-cyanopyridin-3-yl)-2-fluorophenyl]-2-oxopyrrolidin-3-ylcarbamate

Using Intermediate 18 and 3-bromo-2-cyanopyridine, and the synthetic procedure described for Intermediate 74, the title compound (0.053 g) was prepared.

Mass spectrum: Found: MH⁺ 397

Intermediate 76

(3S)-3-Amino-1-[4-(3-chloropyridin-4-yl)-2-fluorophenyl]pyrrolidin-2-one trifluoroacetate

A solution of Intermediate 11 (0.420 g) and tetrakistriphenylphospine palladium(0) (0.025 g) in degassed dimethoxyethane (20 ml) was purged with nitrogen for 5 min. 3-Chloropyridin-4-ylboronic acid pentahydrate (0.248 g) and degassed 0.5M sodium carbonate (6 ml) were added. The resultant solution was heated at 85° C. for 3 h. The reaction mixture was then concentrated under reduced pressure and partitioned between DCM and water. The separated organic layer was dried (hydrophobic frit) and loaded onto a SPE (SCX-2) column (silica, eluting with methanol then, 1N ammonia/methanol) to give tert-butyl (3S)-1-[4-(2-chloropyridin-4-yl)-2-fluorophenyl]-2-oxopyrrolidin-3-ylcarbamate (0.269 g). This material was then dissolved in DCM (10 ml), trifluoroacetic acid (1 ml) added and the solution stirred at room temperature for 2 h. The solution was concentrated under reduced pressure to give the title compound (0.205 g) as a brown oil.

Mass spectrum: Found: MH⁺ 306

Intermediate 77

tert-Butyl (3S)-1-(2-fluoro-4-pyrimidin-2-ylphenyl)-2-oxopyrrolidin-3-ylcarbamate

Using Intermediate 18, 2-bromopyrimidine, and the synthetic procedure described for Intermediate 74, provided the title compound.

Mass spectrum: Found: MH⁺ 372

Intermediate 78

tert-Butyl (3S)-1-[4-(3-chloropyridin-2-yl)-2-fluorophenyl]-2-oxopyrrolidin-3-ylcarbamate

A mixture of Intermediate 18 (0.25), tetrakistriphenylphospinepalladium(0) (0.025 g), 2,3-dichloropyridine (0.074 g), 2M potassium phosphate (0.5 ml) and toluene (1.5 ml) was heated at 80° C. for 18 h. The reaction mixture was diluted with DCM and dried (using a hydrophobic frit). The crude solution was purified by SPE (SCX-2, eluting with methanol, then 0.5M ammonia in methanol) to give the title compound (0.086 g) as a brown gum.

Mass spectrum: Found: MH⁺ 406

Intermediate 79

Ethyl 2-(5-chlorothien-2-yl)-2-hydroxypropane-1-sulfonate

A solution of ethyl methanesulphonate (4.97 g) in THF (20 ml) was added dropwise to a solution of lithium hexamethyldisilylamine (42.0 ml of 1M solution in THF plus 20 ml of THF) at −78° C. under nitrogen, and the solution was stirred for 30 min. A solution of 2-acetyl-5-chlorothiophene (6.75 g) in THF (70 ml) was added to this over 15 min and the temperature maintained at −78° C. for 90 min. The reaction was quenched with saturated aqueous ammonium chloride and the mixture extracted with ethyl acetate. The combined organic fractions were washed with brine; dried (over magnesium sulphate) and concentrated under reduced pressure to afford a crude oil that was purified by Biotage™ chromatography (silica, eluting with ether-cyclohexane 1:3) to give the title compound (10.9 g) as a colourless oil.

¹H NMR (CDCl₃): δ6.79(1H, d), 6.73(1H, d), 4.26(2H, m), 4.14(1H, s), 3.32(1H, d), 3.52(1H, d), 1.8(3H, s), 1.36(3H, t) ppm.

Intermediate 80

Ethyl (1E)-2-(5-chlorothien-2-yl)prop-1-ene-1-sulfonate

A solution of Intermediate 79 (10.9 g) in DCM (300 ml) was cooled to 0° C. under nitrogen, to which was added methanesulphonic acid (15.0 ml) in a dropwise fashion. After stirring for 90 min, saturated aqueous sodium bicarbonate was added, followed by water and brine. The layers were separated and the aqueous layer back extracted with DCM; the organic fractions were combined, washed with brine and dried (over magnesium sulphate) and concentrated under reduced pressure. The crude mixture was ourified using Biotage™ chromatography (silica, eluting chloroform and 15% tert-butylmethyl ether in cyclohexane) to give the title compound (2.9 g) as a white crystalline solid.

¹H NMR (CDCl₃): δ7.16(1H, d), 6.92(1H, d), 6.47(1H, d) 4.26(2H, q), 2.50(3H, d), 1.42 (3H, t) ppm.

Intermediate 81

(1E)-2-(5-Chlorothien-2-yl)prop-1-ene-1-sulfonyl Chloride

Tetrabutylammonium iodide (4.03 g) was added to a solution of Intermediate 80 (2.9 g) in acetone (180 ml) under nitrogen and the solution heated under reflux for 17 h. The solution was cooled and concentrated under reduced pressure to produce a yellow-brown solid. This was stirred in phosphorus oxycloride (30 ml) at room temperature for 3.5 h, after which the volatiles were concentrated under reduced pressure and the residue co-evaporated twice with toluene. The residue was purified using Biotage™ chromatography (silica, eluting with, cyclohexane and cyclohexane:diethyl ether 1:1) to give the title compound (2.1 g) as a yellow crystalline solid.

¹H NMR (CDCl₃): δ7.31(1H, d), 6.99(1H, d), 6.96(1H, q), 2.64(3H, d) ppm.

Intermediate 82

tert-Butyl(1S)-1-{[(2-fluoro-4-nitrophenyl)amino]carbonyl}-3-hydroxypropyl Carbamate

Using the synthetic procedure described for Intermediate 8, the title compound was prepared.

Mass spectrum: Found: MH⁺ 358

The following were prepared similarly:

Intermediate 83

tert-Butyl(1S)-1-{[(4-cyano-2-fluorophenyl)amino]carbonyl}-3-hydroxypropyl Carbamate

Mass spectrum: Found: MH⁺ 338

Intermediate 84

tert-Butyl (1S)-1-{[(2,4-dichlorophenyl)amino]carbonyl}-3-hydroxypropylcarbamate

Mass spectrum: Found: MH⁺ 363

Intermediate 85

tert-Butyl (1S)-1-{[(4-tert-butyl-1,3-thiazol-2-yl)amino]carbonyl}-3-hydroxypropylcarbamate

Mass spectrum: Found: MH⁺ 357

Intermediate 86

tert-Butyl (1S)-1-({[4-(benzyloxy)phenyl]amino}carbonyl)-3-hydroxypropylcarbamate

Mass spectrum: Found: MH⁺ 400

Intermediate 87

tert-Butyl (1S)-1-({[4-(dimethylamino)phenyl]amino}carbonyl)-3-hydroxypropylcarbamate

Mass spectrum: Found: MH⁺ 337

Intermediate 88

tert-Butyl (1S)-1-{[(4-tert-butylphenyl)amino]carbonyl}-3-hydroxypropylcarbamate

Mass spectrum: Found: MH⁺=350

Intermediate 89

tert-Butyl (1S)-1-[(2,3-dihydro-1H-inden-5-ylamino)carbonyl]-3-hydroxypropylcarbamate

Mass spectrum: Found: MH⁺ 334

Intermediate 90

tert-Butyl (1S)-3-hydroxy-1-{[(4-phenoxyphenyl)amino]carbonyl}propylcarbamate

Mass spectrum: Found: MH⁺ 386

Intermediate 91

tert-Butyl (1S)-3-hydroxy-1-[(1,3-thiazol-2-ylamino)carbonyl]propylcarbamate

Mass spectrum: Found: MH⁺ 302

Intermediate 92

tert-Butyl (1S)-1-[(1,3-benzothiazol-2-ylamino)carbonyl]-3-hydroxypropylcarbamate

Mass spectrum: Found: MH⁺ 352

Intermediate 93

tert-Butyl (1S)-1-{[(3-fluoro-4-morpholin-4-ylphenyl)amino]carbonyl}-3 Hydroxypropylcarbamate

Mass spectrum: Found: MH⁺ 398

Intermediate 94

tert-Butyl(1S)-3-hydroxy-1-{[(pyrazin-2-yl)amino]carbonyl}propylcarbamate:

Mass spectrum: Found: MH⁺ 297

H.p.l.c. (1) R_{T} 2.12 min

Intermediate 95

tert-Butyl (3S)-1-(2-fluoro-4-nitrophenyl)-2-oxopyrrolidin-3-ylcarbamate

Using the synthetic procedure described for Intermediate 10, the title compound was prepared.

Mass spectrum: Found: MH⁺ 340

The following were also prepared similarly:

Intermediate 96

tert-Butyl (3S)-1-(4-cyano-2-fluorophenyl)-2-oxopyrrolidin-3-ylcarbamate

Mass spectrum: Found: MH⁺ 320

Intermediate 97

tert-Butyl (3S)-1-(2,4-dichlorophenyl)-2-oxopyrrolidin-3-ylcarbamate

Mass spectrum: Found: MH⁺ 345

Intermediate 98

tert-Butyl (3S)-1-(4-tert-butyl-1,3-thiazol-2-yl)-2-oxopyrrolidin-3-ylcarbamate

Mass spectrum: Found: MH(-Boc)⁺ 240

Intermediate 100

tert-Butyl (3S)-1-(4-tert-butylphenyl)-2-oxopyrrolidin-3-ylcarbamate

Mass spectrum: Found: MH⁺ 333

Intermediate 101

tert-Butyl (3S)-1-(2,3-dihydro-1H-inden-5-yl)-2-oxopyrrolidin-3-ylcarbamate

Mass spectrum: Found: MH(-Boc)⁺ 217

Intermediate 102

tert-Butyl (3S)-2-oxo-1-(4-phenoxyphenyl)pyrrolidin-3-ylcarbamate

Mass spectrum: Found: MH(-Boc)⁺ 269

Intermediate 103

tert-Butyl (3S)-2-oxo-1-(1,3-thiazol-2-yl)pyrrolidin-3-ylcarbamate

Mass spectrum: Found: MH⁺ 284

Intermediate 104

tert-Butyl (3S)-1-(1,3-benzothiazol-2-yl)-2-oxopyrrolidin-3-ylcarbamate

Mass spectrum: Found: MH⁺ 334

Intermediate 105

tert-Butyl (3S)-1-(2-fluoro-4-isopropenylphenyl)-2-oxopyrrolidin-3-ylcarbamate

To a solution of 2-bromopropene (0.18 ml) in anhydrous THF (3 ml) cooled to −78° C. under nitrogen, a solution of n-butyl lithium (2.5M in hexanes, 0.86 ml) was added slowly. The mixture was stirred for a further 15 min before a solution of zinc chloride (1M in diethyl ether, 2.14 ml) was added slowly. This resulting solution was stirred for a further 30 min at −78° C. under nitrogen before it was added to a solution of Intermediate 11 (0.300 g) and dichlorobis(triphenylphosphine)palladium(II) (0.060 g) in anhydrous THF (3.5 ml) cooled to −78° C. The reaction mixture was allowed to warm to ambient temperature and stirred for a further 20 h. The reaction mixture was concentrated under reduced pressure and the residue partitioned between aqueous ammonium chloride and DCM. The organic phases were concentrated under reduced pressure and the resulting crude product was purified by Biotage™ chromatography, (silica, eluting with cyclohexane:ethyl acetate 4:1 to 2:1) followed by mass directed preparative h.p.l.c. to give the title compound (120 mg) as an off-white solid.

Mass spectrum: Found: MH⁺ 335

H.p.l.c. (1) Rt 3.19 min

Intermediate 106

tert-Butyl (3S)-1-[2-fluoro-4-(1H-imidazol-1-yl)phenyl]-2-oxopyrrolidin-3-ylcarbamate

A mixture of Intermediate 11 (0.420 g), 1H-imidazole (0.068 g), copper (I) iodide (0.0048 g), potassium phosphate (0.446 g) and trans diaminocyclohexane in dioxan (2 ml) were heated at 110° C. under nitrogen for 43 h. The reaction mixture was partitioned between DCM and water. The organic phase was dried (hydrophobic frit) and loaded onto a SPE column (eluting with DCM, methanol and finally 0.5M ammonia/methanol) to give an impure sample of the title compound. Further purification on SPE (Silica, eluting with DCM, chloroform, diethyl ether) to give the title compound (0.05 g) as a white solid.

Mass spectrum: Found: MH⁺ 361

H.p.l.c. (1) Rt 2.17 min

Using similar chemistry, the following were prepared:

Intermediate 107

tert-Butyl (3S)-1-[2-fluoro-4-(4-methyl-1H-imidazol-1-yl)phenyl]-2-oxopyrrolidin-3-ylcarbamate

Mass spectrum: Found: MH⁺ 375

Intermediate 108

tert-Butyl (3S)-1-[2-fluoro-4-(1H-pyrazol-1-yl)phenyl]-2-oxopyrrolidin-3-yl Carbamate

Mass spectrum: Found: MH⁺ 361

Intermediate 109

tert-Butyl (3S)-1-(pyrazin-2-yl)-2-oxopyrrolidin-3-ylcarbamate

Mass spectrum: Found: MH⁺ 279

Intermediate 110

(3S)-3-Amino-1-(5-iodopyridin-2-yl)pyrrolidin-2-one Dihydrochloride

Using the synthetic procedure described for Intermediate 24, the title compound was prepared.

Mass spectrum: Found: MH⁺ 304

Using similar chemistry, the following were prepared:

### Example 111

### (3S)-3-Amino-1-(2-fluoro-4-nitrophenyl)pyrrolidin-2-one Hydrochloride

Mass spectrum: Found: MH⁺ 240

### Example 112

### 4-[(3S)-3-Amino-2-oxopyrrolidin-1-yl]-3-fluorobenzonitrile Hydrochloride

Mass spectrum: Found: MH⁺ 220

Intermediate 113

(3S)-3-Amino-1-(2-fluoro-4-isopropenylphenyl)pyrrolidin-2-one

Mass spectrum: Found: M⁺ 235

Intermediate 114

4-N-(3S)-3-Amino-1-(pyrazin-2-yl)pyrrolidin-2-one Dihydrochloride

Mass spectrum: Found: MH⁺ 179

Intermediate 115

(3S)-3-Amino-1-(3-fluoro-4-morpholin-4-ylphenyl)pyrrolidin-2-one

A mixture of diisopropylazodicarboxylate (0.288 g) and tri-n-butylphosphine (0.45 ml) in dry THF (2 ml) was stirred and room temperature under nitrogen for 5 min. This solution was then added dropwise to a solution of tert-butyl (1S)-1-{[(3-fluoro-4-morpholin-4-ylphenyl)amino]carbonyl}-3-hydroxypropylcarbamate (0.379 g) in dry THF (4 ml) and stirred for 20 h at ambient temperature. The mixture was cobentrated under reduced pressure to give a creamy white solid (1.09 g) which was treated with DCM/TFA 1:1 (9 ml). After standing at room temperature for 3.5 h, the reaction mixture was concentrated under reduced pressure to give an oil and basified with saturated aqueous sodium bicarbonate solution. Extraction with DCM gave a pale brown oil (0.913 g). This crude product was dissolved in methanol, loaded onto a SCX-2 ion-exchange cartridge (eluting with methanol and concentrated aqueous ammonia/methanol 1:9) to give the title compound (0.25 g) as a white solid.

Mass spectrum: Found: MH⁺ 280

Intermediate 116

4-N(3S)-3-Amino-1-(pyrazin-2-yl)pyrrolidin-2-one Dihydrochloride

Mass spectrum: Found: MH⁺ 179

Intermediate 117

tert-Butyl (3S)-1-{4-[(dimethylamino)carbonyl]-2-fluorophenyl}-2-oxopyrrolidin-3-ylcarbamate

A solution of tert-butyl (3S)-1-(2-fluoro-4-iodophenyl)-2-oxopyrrolidin-3-ylcarbamate (0.6 g) in dry N′N-dimethylformamide (8 ml) was treated with dimethylamine (2N in tetrahydrofuran) (3.56 ml) and bis(triphenylphosphine)palladium(II) chloride (0.06 g). Carbon monoxide gas was bubbled through the mixture for 10 min and the reaction was then heated to 80° C., for 18 h, under positive carbon monoxide pressure. The cooled reaction was concentrated under reduced pressure, partitioning the residue between ethyl acetate and water. The separated organic layer was dried (over magnesium sulphate) and concentrated under reduced pressure to give crude material which was dissolved in minimum DCM and loaded onto pre-conditioned Silica phase SPE (eluting with cyclohexane: ethylacetate 10:1, 5:2 and neat ethylacetate) to give the title compound (0.188 g) as a white powder.

Mass spectrum: Found: MH⁺ 366

Using similar chemistry, the following were prepared:

Intermediate 118

tert-Butyl (3S)-1-[2-fluoro-4-(pyrrolidin-1-ylcarbonyl)phenyl]-2-oxopyrrolidin-3-ylcarbamate.

Mass spectrum: Found: MH⁺ 392

Intermediate 119

tert-Butyl (3S)-1-[5-(aminocarbonyl)pyridin-2-yl]-2-oxopyrrolidin-3-ylcarbamate

Mass spectrum: Found: MH⁺ 321

Intermediate 120

Di(tert-butyl)(3S)-1-[4-(aminocarbonyl)-2-fluorophenyl]-2-oxopyrrolidin-3-ylimidodicarbonate

Mass spectrum: Found: MH⁺ 438

Intermediate 121

tert-Butyl (3S)-1-{2-fluoro-4-[(methylamino)carbonyl]phenyl}-2-oxopyrrolidin-3-ylcarbamate

Mass spectrum: Found: MH⁺ 352

Intermediate 122

tert-Butyl (3S)-1-(2-fluoro-4-{[isopropyl(methyl)amino]carbonyl}phenyl)-2-oxopyrrolidin-3-ylcarbamate

Mass spectrum: Found: MH⁺ 394

Intermediate 123

(3S)-3-Amino-1-[2-fluoro-4-(pyrrolidin-1-ylcarbonyl)phenyl]pyrrolidin-2-one

Mass spectrum: Found: MH⁺ 292

Intermediate 124

6-[(3S)-3-Amino-2-oxopyrrolidin-1-yl]nicotinamide Hydrochloride

This material was used in crude form in the next stage of the synthetic sequence.

Intermediate 125

4-[(3S)-3-Amino-2-oxopyrrolidin-1-yl]-3-fluorobenzamide Hydrochloride

This material was used in crude form in the next stage of the synthetic sequence.

Intermediate 126

4-[(3S)-3-Amino-2-oxopyrrolidin-1-yl]-3-fluoro-N-methylbenzamide Hydrochloride

Mass spectrum: Found: MH⁺ 252

Intermediate 127

4-[(3S)-3-Amino-2-oxopyrrolidin-1-yl]-3-fluoro-N,N-dimethylbenzamide Hydrochloride

Mass spectrum: Found: MH⁺ 266

Intermediate 128

4-[(3S)-3-Amino-2-oxopyrrolidin-1-yl]-3-fluoro-N-isopropyl-N-methylbenzamide Hydrochloride

Mass spectrum: Found: MH⁺ 294

Intermediate 129

Di(tert-butyl) (3S)-1-(2-fluoro-4-iodophenyl)-2-oxopyrrolidin-3-ylimidodicarbonate

tert-Butyl (3S)-1-(2-fluoro-4-iodophenyl)-2-oxopyrrolidin-3-yl carbamate (1.0 g), suspended in dry acetonitrile (10 ml) at 0° C. was treated with di-tert-butyl dicarbonate (0.571 g) in dry acetonitrile (2.5 ml) and 4-(dimethylamino)pyridine (0.05 g). The reaction was warmed to ambient temperature under nitrogen and stirred for 3.5 h. More di-tert-butyl dicarbonate (0.571 g) in dry acetonitrile (2.5 ml) and 4-(dimethylamino)pyridine (0.05 g) were added to the mixture allowing to stir for 18 h under nitrogen. Solvent was removed under reduced pressure, partitioning the residue between ethyl acetate and saturated sodium bicarbonate solution. The separated organic layer was washed with water, dried (over magnesium sulphate) and concentrated under reduced pressure. The residue was dissolved in minimum DCM and loaded onto preconditioned SPE (Silica, eluting with cyclohexane: ethylacetate 20:1 to 9:1) to give the title compound (0.991 g) as white solid.

Mass spectrum found MH⁺ 521

Intermediate 130

tert-Butyl (3S)-1-(4-acetyl-2-fluorophenyl)-2-oxopyrrolidin-3-ylcarbamate

A de-gassed solution of tert-butyl (3S)-1-(2-fluoro-4-iodophenyl)-2-oxopyrrolidin-3-ylcarbamate (1.05 g) in dry N′N-dimethylformamide (20 ml) was treated sequentially with sodium carbonate (0.42 g), triethylamine(0.67 ml), butyl vinyl ether (1.62 ml), 1,3-bis(diphenylphosphino)propane (0.124 g) and palladium(II) acetate (0.034 g). The mixture was heated to 80° C. under nitrogen for 7 h, allowing to cool and stir for 18 h. Solvent was removed under reduced pressure and the crude residue treated with 0.1% formic acid: water (10 ml) and acetonitrile (10 ml). The mixture was stirred at ambient temperature for 4 h before concentrating under reduced pressure. The residue was dissolved in minimum DCM and loaded onto pre-conditioned SPE (Silica, eluting with cyclohexane: ethylacetate 5:1 to neat ethylacetate) to give the title compound (0.362 g) as a yellow powder

Mass spectrum: Found: MH⁺ 337

Using similar chemistry, the following were prepared:

Intermediate 131

tert-Butyl (3S)-1-(5-acetylpyridin-2-yl)-2-oxopyrrolidin-3-ylcarbamate

Mass spectrum: Found: MH³⁰ 320

Intermediate 132

(3S)-1-(4-Acetyl-2-fluorophenyl)-3-aminopyrrolidin-2-one Hydrochloride

Mass spectrum: Found: MH⁺ 237

Intermediate 133

(3S)-1-(5-Acetylpyridin-2-yl)-3-aminopyrrolidin-2-one Hydrochloride

Mass spectrum: Found: MH⁺ 220

Intermediate 134

(E)-N-{(3S)-1-[4-(1-Bromoethyl)-2-fluorophenyl]-2-oxopyrrolidin-3yl}-2-(5-chlorothien-2-yl)ethenesulfonamide

A solution of (E)-2-(5-chlorothien-2-yl)-N-{(3S)-1-[2-fluoro-4-(1-hydroxyethyl)phenyl]-2-oxopyrrolidin-3-yl}ethenesulfonamide Example 135 (0.149 g) in dry DCM (6 ml) at 0° C. was treated with carbon tetrabromide (0.136 g), stirring for 5 min. To the mixture was added triphenylphosphine (0.106 g) in portions and the reaction stirred at 0° C. for 2 h before more carbon tetrabromide (0.136 g) and triphenylphosphine (0.106 g) were added. The reaction was warmed up to ambient temperature and stirred for 18 h under nitrogen. The mixture was diluted with DCM and washed with water. The separated organic layer was dried (hydrophobic frites) and concentrated under reduced pressure, to a small volume, and loaded onto pre-conditioned SPE (Silica, eluting with cyclohexane: ethylacetate 10:1 to 2:1) to give the title compound (0.09 g) as a beige solid.

Mass spectrum: Found: MH⁻ 506

Intermediate 135

(E)-2-(5-Chlorothien-2-yl)-N-((3S)-1-{4-[1-(diformylamino)ethyl]-2-fluorophenyl}-2-oxopyrrolidin-3-yl)ethenesulfonamide

A solution of Intermediate 134 (0.09 g), in dry N′N-dimethylformamide (4 ml), was treated with sodium diformamide (0.019 g) and then heated to 50° C. under nitrogen for 3.5 h. The reaction was cooled to ambient temperature and the solvent removed under reduced pressure, partitioning the residue between DCM and water. The separated organic layer was dried (hydrophobic frits) and re-concentrated under reduced pressure to give the title compound (0.075 g) as an orange gum

Mass spectrum: Found: MH⁻ 498

Intermediate 136

tert-Butyl(3S)-1-[2-fluoro-4-(isopropylamino)phenyl]-2-oxopyrrolidin-3-ylcarbamate

A solution of tert-butyl (3S)-1-(4-amino-2-fluorophenyl)-2-oxopyrrolidin-3-ylcarbamate (0.329 g) in ethyl alcohol (4 ml) was treated with dry acetone (0.118 ml) and titanium (IV) isopropoxide (0.106 ml) allowing the mixture to stir at ambient temperature for 18 h. Sodium borohydride (0.027 g) was added in portions and the reaction was allowed to stir for a further 3 h, before quenching with 35% aqueous ammonia (1 ml). The resulting precipitate was removed by filtration and the filtrate concentrated under reduced pressure. The crude material was taken up in minimum DCM and loaded onto pre-conditioned SPE (Silica, eluting with cyclohexane: ethylacetate 10:1 to 1:2) to give the title compound (0.080 g) as a yellow powder.

Mass spectrum: Found: MH⁺ 352

Intermediate 137

tert-Butyl (3S)-1-[2-fluoro-4-(isobutyrylamino)phenyl]-2-oxopyrrolidin-3-ylcarbamate

tert-Butyl (3S)-1-(4-amino-2-fluorophenyl)-2-oxopyrrolidin-3-ylcarbamate (0.133 g) was dissolved in DCM (4 ml) and the mixture cooled to 0° C. using a salt and ice bath. 2-Methylpropanoyl chloride (0.041 ml) was added dropwise, and the mixture left for 4 h and then concentrated under reduced pressure to give a white solid. The solid was purified by SPE (benzenesulphonic acid on silica, methanol elution) to give the title compound (0.086 g) as a white solid.

Mass spectrum: Found: MH⁺ 380

Using similar chemistry, the following were prepared:

Intermediate 138

tert-Butyl (3S)-1-[4-(acetylamino)-2-fluorophenyl]-2-oxopyrrolidin-3-ylcarbamate

Mass spectrum: Found: MH⁺ 352

Intermediate 139

tert-Butyl (3S)-1-[2-fluoro-4-(propionylamino)phenyl]-2-oxopyrrolidin-3-ylcarbamate

Mass spectrum: Found: MH⁺ 366.2

Intermediate 140

tert-Butyl (3S)-1-{2-fluoro-4-[formyl(isopropyl)amino]phenyl}-2-oxopyrrolidin-3-ylcarbamate

98% Formic acid (0.344 ml) was added to acetic anhydride (0.718 ml) at 0° C. The mixture was heated to 60° C. for 2 h, cooled to ambient temperature and diluted with dry tetrahydrofuran (6 ml). The reaction was cooled again to 0° C. and treated with a solution of tert-butyl(3S)-1-[2-fluoro-4-(isopropylamino)phenyl]-2-oxopyrrolidin-3-ylcarbamate (0.10 g) in dry tetrahydrofuran (6 ml) in dropwise manor, before allowing to warm up to room temperature and stir for 2 h. The solvent was removed under reduced pressure to give the title compound (0.10 g) as a pink gum.

Mass spectrum: Found: MNH₄⁺ 297

Intermediate 141

N-{4-[(3S)-3-Amino-2-oxopyrrolidin-1-yl]-3-fluorophenyl}-2-methylpropanamide

tert-Butyl (3S)-1-[2-fluoro-4-(isobutyrylamino)phenyl]-2-oxopyrrolidin-3-ylcarbamate (0.086 g) was dissolved in methanol (2 ml) and cooled to 0° C. using a salt/ice bath. Acetyl chloride (1 ml) was added dropwise, and the mixture left to warm to room temperature. The mixture was stirred for 1.5 h then concentrated under a stream of nitrogen to give the title compound (0.063 g) as a clear gum.

Mass spectrum: Found: MH⁺ 280

Using similar chemistry, the following were prepared:

Intermediate 142

N-{4-[(3S)-3-Amino-2-oxopyrrolidin-1-yl]-3-fluorophenyl}acetamide

Mass spectrum: Found: MH⁺ 252.2

Intermediate 143

N-{4-[(3S)-3-Amino-2-oxopyrrolidin-1-yl]-3-fluorophenyl}propanamide

Mass spectrum: Found: MH⁺ 266

Intermediate 144

4-[(3S)-3-Amino-2-oxopyrrolidin-1-yl]-3-fluorophenyl(isopropyl)formamide Hydrochloride

Mass spectrum: Found: MH⁺ 297

Intermediate 145

2-(2-Bromoethyl)-5-chlorothiophene

To a solution of 2-(5-chloro-2-thienyl)-ethanol* (12.2 g) and triphenylphosphine (21.4 g) in anhydrous THF (150 ml) at 0° C. was added carbon tetrabromide (27.5 g). The reaction was stirred at 5° C. for 15 min then at room temperature for 2.5 h. Ether was added and the reaction was then filtered and the filtrate concentrated. The resultant residue was purified by flash column chromatography (silica, eluting with cyclohexane:DCM 8:1) to give the title compound (15 g) as an oil.

¹H NMR in CDCl₃: δ3.27 (2H, t, J 8 Hz), 3.53 (2H, t, J 8 Hz), 6.66 (1H, d, J 4 Hz), 6.76 (1H, d, J 4 Hz) ppm.

*Schick et al., J. Amer. Chem. Soc., 70, 1948, 1646.

Intermediate 146

2-(5-Chlorothien-2-yl)ethanesulfonyl Chloride

To a stirred solution of Intermediate 145 (14 g) in acetone (125 ml) was added an aqueous solution of sodium sulphite (10.5 g in 125 ml of water). The reaction was heated at reflux for 18 h then concentrated to yield a pink solid, which was dried under vacuum at 50° C. for 18 h. A suspension of the salt in phosphorus oxychloride (90 ml) was heated at 150° C. for 2.5 h. The reaction was concentrated and DCM and water added to the resultant residue. The organic portion was collected, concentrated and the resultant oil purified by flash column chromatography (silica, eluting with petroleum ether:toluene 7:3) to give the title compound (12.47 g) as a brown oil.

¹H NMR in CDC₃: δ3.70 (2H, m), 3.22 (2H, m), 6.72 (1H, d, J 4 Hz), 6.79 (1H, d, J 4 Hz) ppm.

Intermediate 147

tert-Butyl (3S)-1-(4-amino-2-fluorophenyl)-2-oxopyrrolidin-3-ylcarbamate

A solution of Intermediate 95 (2.50 g) in ethanol (220 ml) was added under vacuum to 10% palladium on carbon (1.54 g , 50% wet). The resulting suspension was stirred under an atmosphere of hydrogen for 16 h, then filtered through Celite™ washing thoroughly with ethanol. The combined filtrates were evaporated under reduced pressure to give a grey foam which was purified by SPE-SCX (eluting with 10% 0.88 (specific gravity) aqueous ammonia in methanol) to give the title compound (1.985 g) as a white foam.

Mass spectrum: Found: MH⁺ 310

Intermediate 148

tert-Butyl (3S)-1-{2-fluoro-4-[(methylsulfonyl)amino]phenyl}-2-oxopyrrolidin-3-ylcarbamate

A solution of Intermediate 147 (0.1 g) in anhydrous DCM (1 ml) was cooled to 0° C. under nitrogen and treated sequentially with anhydrous pyridine (0.06 ml) and methanesulphonyl chloride (0.03 ml) then stirred for 2 h at 0° C. (solution colour change noted during this time:clear to yellow to orange to pink). The solution was allowed to warn to room temperature, diluted with DCM and washed with saturated sodium bicarbonate. The yellow organic layer was dried (hydrophobic frit) and evaporated under nitrogen to give a pink solid which was purified by SPE (silica, eluting with DCM then ethyl acetate) to give the title compound (0.068 g) as a white solid.

Mass spectrum: Found: MH⁺ 388

Intermediate 149

N-{4-[(3S)-3-Amino-2-oxopyrrolidin-1-yl]-3-fluorophenyl}methanesulfonamide Hydrichloride

A solution of Intermediate 148 (0.066 g) in methanol (5 ml) was treated with acetyl chloride (0.5 ml) and stirred under an atmosphere of nitrogen for 6 hours then stood for 48 hr. The solution was evaporated under reduced pressure to give a white foam which was purified using SPE (C18, eluting with water) to give the hydrochloride salt as a white foam (0.055 g). The hydrochloride salt was applied to SPE (silica, eluting with DCM:methanol:0.88(SG) aqueous ammonia 100:10:1) gave the title compound (0.033 g) as a clear glass.

Mass spectrum: Found: MH+ 288

References

1. Klimkowski, Valentine Joseph; Kyle, Jeffrey Alan; Masters, John Joseph; Wiley, Michael Robert. PCT Int. Appl. (2000), WO 0039092.
   In Vitro Assay for Inhibition of Factor Xa (1)

Compounds of the present invention (Examples 2, 19, 20, 21, 22, 23, 52, 73, 83, 85, 89, 90, 102, 105, 123, 124, 125, 127) were tested for their Factor Xa inhibitory activity as determined in vitro by their ability to inhibit human Factor Xa in a chromogenic assay, using N-α-benzyloxycarbonyl-D-Arg-Gly-Arg-p-nitroanilide as the chromogenic substrate. Compounds were diluted from a 10 mM stock solution in dimethylsulfoxide at appropriate concentrations. Assay was performed at room temperature using buffer consisting of: 50 mM Tris-HCl, 150 mM NaCl, 5 mM CaCl₂, pH 7.4. containing human Factor Xa (final conc. of 0.0015 U,ml⁻¹). Compound and enzyme were preincubated for 15 min prior to addition of the substrate (final conc. of 200 μM). The reaction was stopped after 30 min with the addition of soybean trypsin inhibitor or H-D-PHE-PRO-ARG-Chloromethylketone. BioTek EL340 or Tecan Spectrafluoro Plus plate readers were used to monitor the absorbance at 405 nm. To obtain IC₅₀ values the data were analysed using ActivityBase® and XLfit®.

In Vitro Assay for Inhibition of Factor Xa (2)

All other compounds of the present invention were tested for their Factor Xa inhibitory activity as determined in vitro by their ability to inhibit human Factor Xa in a fluorogenic assay, using Rhodamine 110, bis-(CBZ-glycylglycyl-L-arginine amide as the fluorogenic substrate. Compounds were diluted from a 10 mM stock solution in dimethylsulfoxide at appropriate concentrations. Assay was performed at room temperature using buffer consisting of: 50 mM Tris-HCl, 150 mM NaCl, 5 mM CaCl₂, pH 7.4. containing human Factor Xa (final conc. Of 0.0003U.ml−1). Compound and enzyme were preincubated for 15 min prior to addition of the substrate (final conc. of 10 μM). The reaction was stopped after 3 hrs with the addition of H-D-PHE-PRO-ARG-Chloromethylketone. An LJL-Analyst fluorimeter was used to monitor fluorescence with 485 nm excitation/535 nm emission. To obtain IC₅₀ values the data were analysed using ActivityBase® and XLfit®.

Calculation of Ki values:

Ki=IC₅₀/(1+[Substrate]/Km)

The Ki value for the above assay can be obtained by dividing the IC₅₀ value by 1.6.

All of the synthetic Example compounds tested by one of the above described in vitro assays for Factor Xa exhibited inhibitory activity.

Preferably compounds have a Ki value of less than 1 μM (Examples 1, 2, 3, 4, 5, 6, 7, 8, 9, 11, 12, 13, 14, 15, 16, 17, 18, 19, 20, 21, 23, 24, 25, 26, 27, 28, 29, 30, 31, 32, 33, 34, 36, 37, 38, 39, 40, 41, 42, 43, 44, 45, 46, 47, 48, 49, 50, 51, 53, 55, 56, 57, 58, 60, 61, 63, 63, 64, 65, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 110, 111, 112, 113, 117, 118, 120, 121, 122, 123, 125, 128, 129, 132, 133, 135, 136, 137, 138, 139, 140, 141, 142, 143, 144). More preferably, compounds have an Ki value of less than 200 nM (Examples 1, 2, 3, 4, 5, 7, 8, 11, 12, 13, 14, 15, 16, 17, 18, 19, 20, 21, 24, 25, 26, 27, 28, 29 30, 31, 32, 33, 36, 37, 38, 39, 40, 41, 42, 43, 44, 45, 46, 47, 48, 49, 50, 51, 53, 55, 56, 57, 58, 60, 61, 62, 63, 64, 65, 67, 68, 69, 70, 71, 72, 74, 75, 76, 77, 78, 79, 80, 82, 83, 84, 85, 86, 87, 88, 89, 91, 92, 93, 94, 95, 96, 97, 98, 99, 100, 102, 104, 105, 106, 107, 108, 110, 112, 113, 120, 121, 122, 123, 125, 128, 129, 132, 133, 135, 136, 137, 138, 139, 140, 141, 142, 143, 144), even more preferably compounds have a Ki value of less than 20 nM (1, 2, 3, 4, 5, 7, 8, 11, 12, 13, 14, 15, 16, 17, 18, 19, 20, 21, 24, 25, 26, 27, 28, 29, 30, 36, 37, 38, 39, 40, 41, 42, 43, 44, 45, 46, 47, 48, 49, 50, 53, 55, 57, 62, 64, 70, 72, 75, 76, 77, 78, 79, 80, 82, 83, 84, 85, 86, 87, 88, 91, 92, 93, 95, 96, 97, 100, 104, 107, 110, 112, 120, 121, 122, 128, 129, 132, 133, 136, 137, 139, 140, 141, 142, 143, 144) and most preferably compounds have a Ki value of less than 10 nM(1, 3, 4, 5, 7, 8, 11, 12, 13, 14, 15, 16, 17, 18, 19, 20, 24, 26, 27, 28, 29, 30, 36, 38, 39, 40, 41, 42, 43, 44, 45, 46, 47, 48, 49, 50, 55, 57, 62, 64, 75, 77, 78, 79, 80, 82, 83, 85, 87, 91, 92, 93, 97, 100, 104, 107, 109, 110, 112, 120, 122, 128, 129, 133, 134, 136, 139, 140, 141, 142, 143, 144).

Method for Measurement of Prothrombin Time (PT)

Blood is collected into a sodium citrate solution (ratio 9:1) to give a final concentration of 0.38% citrate. Plasma is generated by centrifugation of citrated blood samples at 1200×g for 20 min at 4° C .

The PT test is performed at 37° C. in plastic cuvettes containing a magnetic ball bearing. 50 μL of citrated plasma and either 25 μL of 2.8% DMSO for control or 25 μL of test compound (dissolved in DMSO and diluted in water and 2.8% DMSO to give 0.4% DMSO final in assay) at a concentration of 7-times the final desired concentration is pippetted into each cuvette. This mixture is incubated for 1 min at 37° C. before adding 100 μL of thromboplastin mixture (comprising lyophilised rabbit thromboplastin and calcium chloride which is reconstituted in distilled water as per manufacturer's [Sigma] instructions). On addition of the thromboplastin mixture, the timer is automatically started and continued until the plasma clotted. The time to clotting was recorded (normal range for human plasma is 10-13 seconds).

Method for Measurement of Prothrombin Time (PT)—Test 2

Blood is collected into a sodium citrate solution (ratio 9:1) to give a final concentration of 0.38% citrate. Plasma is generated by centrifugation of citrated blood samples at 1200×g for 20 min at 4° C.

The PT test is performed at 37° C. in plastic cassettes and using a MCA210 Microsample Coagulation Analyzer (Bio/Data Corporation). For assay, 25 ul of plasma containing test compound at concentrations ranging from 0.1 to 100 uM (made from a 1 mM stock solution in 10% DMSO and plasma) and 25 ul of Thromboplastin C Plus (Dade Berhing) are automatically injected into the cassette. Upon addition of the Thromboplastin C Plus, the instrument determines and records the time to clot (normal range for human plasma is 10-13 seconds).

General Purification and Analytical Methods

LC/MS Method

Analytical HPLC was conducted on a Supelcosil LCABZ+PLUS column (3 μm, 3.3 cm×4.6 mm ID) eluting with 0.1% HCO₂H and 0.01 M ammonium acetate in water (solvent A), and 95% acetonitrile and 0.05% HCO₂H in water (solvent B), using the following elution gradient 0-0.7 minutes 0% B, 0.7-4.2 minutes 0→100% B, 4.2-5.3 minutes 100% B, 5.3-5.5 minutes 100→0% B at a flow rate of 3 ml/minutes (System 1). The mass spectra (MS) were recorded on a Fisons VG Platform mass spectrometer using electrospray positive ionisation [(ES+ve to give MH⁺ and M(NH₄)⁺ molecular ions] or electrospray negative ionisation [(ES−ve to give (M−H)⁻ molecular ion] modes.

¹H nmr spectra were recorded using a Bruker DPX 400 MHz spectrometer using tetramethylsilane as the external standard.

Biotage™ chromatography refers to purification carried out using equipment sold by Dyax Corporation (either the Flash 40i or Flash 150i) and cartridges pre-packed with KPSil.

Mass directed autoprep refers to methods where the material was purified by high performance liquid chromatography on a HPLCABZ+ 5 μm column (5 cm×10 mm i.d.) with 0.1% HCO₂H in water and 95% MeCN, 5% water (0.5% HCO₂H) utilising the following gradient elution conditions: 0-1.0 minutes 5% B, 1.0-8.0 minutes 5→30% B, 8.0-8.9 minutes 30% B, 8.9-9.0 minutes 30→95% B, 9.0-9.9 minutes 95% B, 9.9-10 minutes 95→0% B at a flow rate of 8 ml minutes⁻¹ (System 2). The Gilson 202-fraction collector was triggered by a VG Platform Mass Spectrometer on detecting the mass of interest.

Hydrophobic frits refers to filtration tubes sold by Whatman.

SPE (solid phase extraction) refers to the use of cartidges sold by International Sorbent Technology Ltd.

TLC (thin layer chromatography) refers to the use of TLC plates sold by Merck coated with silica gel 60₂₅₄.

1. A compound of formula (I): 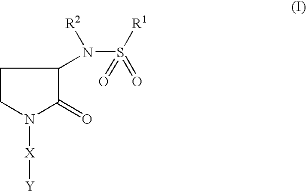
wherein:
R¹ represents a group selected from: 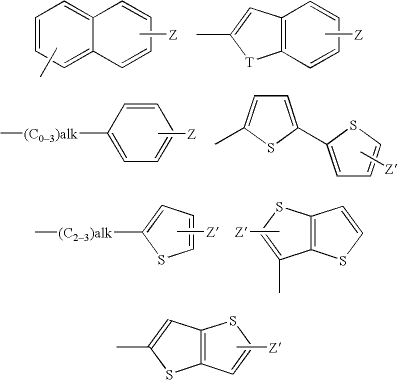
each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alkylene or alkenylene,
T represents S, O or NH;
R² represents hydrogen, —C₁₋₃alkylCONR^{a}R^{b}, —C₁₋₃alkylCO₂C₁₋₄alkyl, —C₁₋₃alkylmorpholino, —CO₂C₁₋₄alkyl, or —C₁₋₃alkylCO₂H;
X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, and —S(O)₂NR^{a}R^{b};
Y represents (i) a substituent selected from hydrogen, halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, —C₀₋₄alkylOR^{d}, —C(O)R^{c}, —C(O)NR^{a}R^{b}, —S(O)ₙR^{c}, or —S(O)₂NR^{a}R^{b}, or (ii) phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙN⁺R^{a}R^{b}CH₂CONH₂, —C₀₋₄alkylOR^{d}, —C(O)R^{c}, —C(O)NR^{a}R^{b}, —S(O)ₙR^{c}, —S(O)₂NR^{a}R^{b}, ═O, oxide to a ring N, -—CHO, —NO₂, and —N(R^{a})(SO₂R^{c});
R^{a} and R^{b} independently represent hydrogen, —C₁₋₆alkyl, or together with the N atom to which they are bonded form a 5-, 6- or 7-membered heterocyclic ring optionally containing an additional heteroatom selected from O, N or S, optionally substituted by C₁₋₄alkyl, and optionally the S heteroatom is substituted by one or two O atoms;
R^{c} represents —C₁₋₆alkyl;
R^{d} represents hydrogen or —C₁₋₆alkyl;
n represents 0-2;
and pharmaceutically acceptable derivatives thereof.

2. A compound according to claim 1 wherein Y represents (i) a substituent selected from hydrogen, halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, —C₀₋₄alkylOR^{d}, —C(O)R^{c}, —C(O)NR^{a}R^{b}, —S(O)ₙR^{c}, or —S(O)₂NR^{a}R^{b}, (ii) phenyl optionally substituted by 0-2 groups selected from: halogen, —CN, —C₁₋₄alkyl, —CF₃, —(CH₂)ₙNR^{a}R^{b}, C₀₋₄alkylOR^{d}, —C(O)R^{c}, —C(O)NR^{a}R^{b}, —S(O)ₙR^{c}, —S(O)₂NR^{a}R^{b}, —CHO, —NO₂, and —N(R^{a})(SO₂R^{c}), (iii) a 5 or 6 membered aromatic or non-aromatic heterocyclic group containing at least one heteroatom selected from O, N or S, each of which is substituted by a group selected from: —S(O)ₙR^{c}, —S(O)₂NR^{a}R^{b}, —NO₂, or —N(R^{a})(SO₂R^{c}), or (iv) when R¹ represents 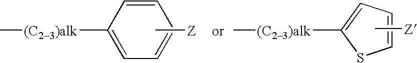
Y represents a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(C H₂)ₙNR^{a}R^{b}, —(CH₂)ₙN⁺R^{a}R^{b}CH₂CONH₂, C₀₋₄alkylOR^{d}, —C(O)R^{c}, —C(O)NR^{a}R^{b}, —S(O)ₙR^{c}, —S(O)₂NR^{a}R^{b}, oxide to a ring N, —CHO, —NO₂, and —N(R^{a})SO₂R^{c}.

3. A compound according to claim 1 wherein: 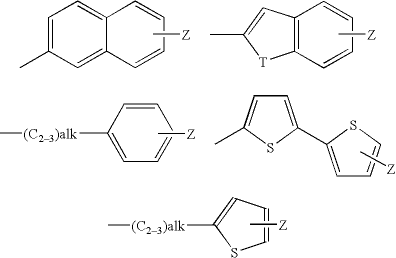
R¹ represents a group selected from:
Z represents an optional substituent halogen,
alk represents alkylene or alkenylene,
T represents S, O or NH;
R² represents hydrogen
X represents phenyl or a 5 or 6 membered aromatic or non-aromatic heterocyclic group containing at least one heteroatom selected from O, N or S, each of which is optionally substituted by 0-2 groups selected from: halogen —CN, —C₁₋₄alkyl, —CF₃, —NR^{a}R^{b}, —(CH₂)ₙOR^{c}, —C(O)R^{c}, —C(O)NR^{a}R^{b}, —S(O)ₙR^{c}, —S(O)₂NR^{a}R^{b};
Y represents (i) a substituent selected from: hydrogen, halogen —CN, —C₁₋₄alkyl, —CF₃, —NR^{a}R^{b}, —(CH₂)ₙOR^{c}, —C(O)R^{c}, —C(O)NR^{a}R^{b}, —(O)ₙR^{c}, —S(O)₂NR^{a}R^{b}, or (ii) phenyl or a 5 or 6 membered aromatic or non-aromatic heterocyclic group containing at least one heteroatom selected from O, N or S, each of which is optionally substituted by 0-2 groups selected from: halogen —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, —S(O)₂NR^{a}R^{b};
R^{a} and R^{b} independently represent hydrogen, —C₁₋₆alkyl or together with the N atom to which they are bonded form a 5-, 6- or 7-membered heterocyclic ring optionally containing an additional heteroatom selected from O, N or S and are optionally substituted by C₁₋₄alkyl, optionally the S heteroatom is substituted by one or two O atoms;
R^{c} represents —C₁₋₆alkyl;
n represents 0-2;
and pharmaceutically acceptable salts and solvates thereof.

4. A compound according to claim 3 wherein Y represents (i) a substituent selected from hydrogen, halogen, —CN, —C₁₋₄alkyl, —CF₃, —NR^{a}R^{b}, —(CH₂)ₙOR^{c}, —C(O)R^{c}, —C(O)NR^{a}R^{b}, —S(O)ₙR^{c}, or —S(O)₂NR^{a}R^{b}, (ii) phenyl optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, and —S(O)₂NR^{a}R^{b}, (iii) a 5 or 6 membered aromatic or non-aromatic heterocyclic group containing at least one heteroatom selected from O, N or S, each of which is substituted by a group selected from: —S(O)ₙR^{c}, —S(O)₂NR^{a}R^{b}, or (iv) when R¹ represents 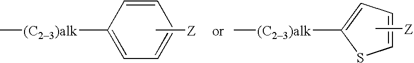
and Z represents an optional substituent halogen, Y represents a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, —(CH₂)ₙNR^{a}R^{b}, —(CH₂)ₙOR^{c}, —C(O)R^{c}, —C(O)NR^{a}R^{b}, —S(O)ₙR^{c}, and —S(O)₂NR^{a}R^{b}.

5. compound according to claim 1 having the formula (IA): 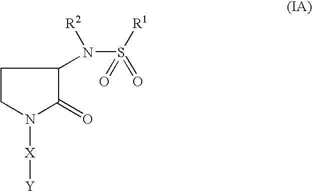
wherein:
R¹ represents a group selected from: 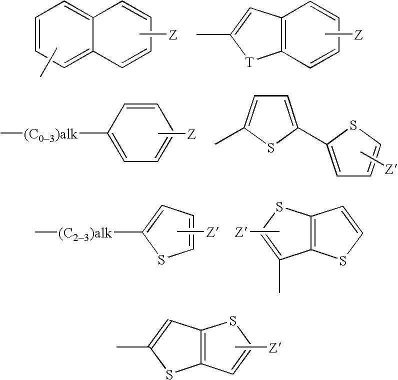
each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alkylene or alkenylene,
T represents S, O or NH;
R² represents hydrogen, —C₁₋₃alkylCONR^{a}R^{b}, —C₁₋₃alkylCO₂C₁₋₄alkyl, —C₁₋₃alkylmorpholino, —CO₂C₁₋₄alkyl, or —C₁₋₃alkylCO₂H;
X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, —C₀₋₄alkylOR^{d}, —C(O)R^{c}, —C(O)NR^{a}R^{b}, —S(O)ₙR^{c}, and —S(O)₂NR^{a}R^{b};
Y represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —CF₃, (CH₂)ₙNR^{a}R^{b}, —(CH₂)ₙN⁺R^{a}R^{b}CH₂CONH₂, C₀₋₄alkylOR^{d}, —C(O)R^{c}, —C(O)NR^{a}R^{b}, —S(O)ₙR^{c}, —S(O)₂NR^{a}R^{b}, ═O, oxide to a ring N, —CHO, —NO₂, and —N(R^{a})(SO₂R^{c});
R^{a} and R^{b} independently represent hydrogen, —C₁₋₆alkyl, or together with the N atom to which they are bonded form a 5-, 6- or 7-membered heterocyclic ring optionally containing an additional heteroatom selected from O, N or S, optionally substituted by C₁₋₄alkyl, and optionally the S heteroatom is substituted by one or two O atoms;
R^{c} represents —C₁₋₆alkyl;
R^{d} represents hydrogen or —C₁₋₆alkyl;
n represents 0-2;
and pharmaceutically acceptable derivatives thereof.

6. A compound according to claim 1 having the formula (IC): 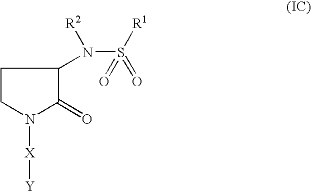
wherein:
R¹ represents a group selected from: 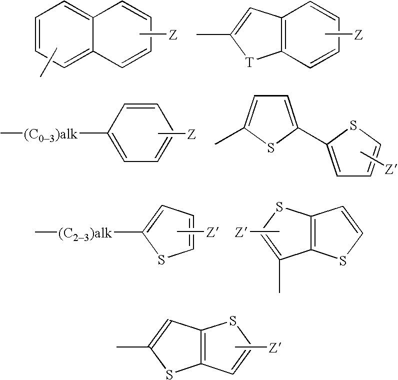
each of which optionally contain a further heteroatom N,
Z represents an optional substituent halogen, —CH₂NH₂, —NR^{a}R^{b} or —CN,
Z′ represents an optional substituent halogen, —CH₂NH₂, or —CN,
alk represents alkylene or alkenylene,
T represents S, O or NH;
R² represents hydrogen, —C₁₋₃alkylCONR^{a}R^{b}, —C₁₋₃alkylCO₂C₁₋₄alkyl, —C₁₋₃alkylmorpholino, —CO₂C₁₋₄alkyl, or —C₁₋₃alkylCO₂H;
X represents phenyl or a 5 or 6 membered aromatic or non-aromatic heterocyclic group containing at least one heteroatom selected from O, N or S, each of which is optionally substituted by 0-2 groups selected from: halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, and —S(O)₂NR^{a}R^{b};
Y represents a substituent selected from hydrogen, halogen, —CN, —C₁₋₄alkyl, —C₂₋₄alkenyl, —CF₃, —NR^{a}R^{b}, —NO₂, —N(C₁₋₄alkyl)(CHO), —NHCOC₁₋₄alkyl, —NHSO₂R^{c}, C₀₋₄alkylOR^{d}, —C(O)R^{c}, —C(O)NR^{a}R^{b}, —S(O)ₙR^{c}, or —S(O)₂NR^{a}R^{b};
R^{a} and R^{b} independently represent hydrogen, —C₁₋₆alkyl, or together with the N atom to which they are bonded form a 5-, 6- or 7-membered heterocyclic ring optionally containing an additional heteroatom selected from O, N or S, optionally substituted by C₁₋₄alkyl, and optionally the S heteroatom is substituted by one or two O atoms;
R^{c} represents —C₁₋₆alkyl;
R^{d} represents hydrogen or —C₁₋₆alkyl;
n represents 0-2;
and pharmaceutically acceptable derivatives thereof.

7. Cancel.

8. A pharmaceutical composition comprising a compound according to claim 1 together with a pharmaceutical carrier and/or excipient.

9. Cancel.

10. A method of treating a patient suffering from a condition susceptible to amelioration by a Factor Xa inhibitor comprising administering a therapeutically effective amount of a compound according to claim 1.
